SEQUENCE LISTING (368 sequences)

SEQ ID NO: 1 - protein, 35 aa, synthetic construct
  REGION 1..35: note = CM18-Penetratin-cys
KWKLFKKIGAVLKVLTTGRQIKIWFQNRRMKWKKC

SEQ ID NO: 2 - protein, 41 aa, synthetic construct
  REGION 1..41: note = TAT-KALA
YGRKKRRQRRRWEAKLAKALAKALAKHLAKALAKALKACEA

SEQ ID NO: 3 - protein, 35 aa, synthetic construct
  REGION 1..35: note = His-CM18-PTD4
HHHHHHKWKLFKKIGAVLKVLTTGYARAAARQARA

SEQ ID NO: 4 - protein, 43 aa, synthetic construct
  REGION 1..43: note = His-LAH4-PTD4
HHHHHHKKALLALALHHLAHLALHLALALKKAYARAAARQARA

SEQ ID NO: 5 - protein, 41 aa, synthetic construct
  REGION 1..41: note = PTD4-KALA
YARAAARQARAWEAKLAKALAKALAKHLAKALAKALKACEA

SEQ ID NO: 6 - protein, 34 aa, synthetic construct
  REGION 1..34: note = EB1-PTD4
LIRLWSHLIHIWFQNRRLKWKKKYARAAARQARA

SEQ ID NO: 7 - protein, 41 aa, synthetic construct
  REGION 1..41: note = His-CM18-PTD4-6Cys
HHHHHHKWKLFKKIGAVLKVLTTGYARAAARQARACCCCCC

SEQ ID NO: 8 - protein, 29 aa, synthetic construct
  REGION 1..29: note = CM18-PTD4
KWKLFKKIGAVLKVLTTGYARAAARQARA

SEQ ID NO: 9 - protein, 35 aa, synthetic construct
  REGION 1..35: note = CM18-PTD4-6His
KWKLFKKIGAVLKVLTTGYARAAARQARAHHHHHH

SEQ ID NO: 10 - protein, 41 aa, synthetic construct
  REGION 1..41: note = His-CM18-PTD4-His
HHHHHHKWKLFKKIGAVLKVLTTGYARAAARQARAHHHHHH

SEQ ID NO: 11 - protein, 30 aa, synthetic construct
  REGION 1..30: note = TAT-CM18
YGRKKRRQRRRCKWKLFKKIGAVLKVLTTG

SEQ ID NO: 12 - protein, 37 aa, synthetic construct
  REGION 1..37: note = FSD5
HHHHHHLLKLWSRLLKLWTQGRRLKAKRAKAHHHHHH

SEQ ID NO: 13 - protein, 34 aa, synthetic construct
  REGION 1..34: note = FSD10
KWKLARAFARAIKKLGGSGGGSYARALRRQARTG

SEQ ID NO: 14 - protein, 27 aa, synthetic construct
  REGION 1..27: note = FSD12
KWKLARAFARAIKKLYARALRRQARTG

SEQ ID NO: 15 - protein, 32 aa, synthetic construct
  REGION 1..32: note = FSD18
LLKLWSRLLKLWTQGGSGGGSGRRLKAKRAKA

SEQ ID NO: 16 - protein, 42 aa, synthetic construct
  REGION 1..42: note = FSD19
HHHHHHLLKLWSRLLKTWTQGRRLKAKSAQASTRQAHHHHHH

SEQ ID NO: 17 - protein, 31 aa, synthetic construct
  REGION 1..31: note = FSD21
HHHHHHFLKIWSRLIKIWTQGRRKGAQAAFR

SEQ ID NO: 18 - protein, 37 aa, synthetic construct
  REGION 1..37: note = FSD23
HHHHHHLLKLWSRLLKEWTQGRRLEAKRAEAHHHHHH

SEQ ID NO: 19 - protein, 31 aa, synthetic construct
  REGION 1..31: note = FSD120
HHHHHHFLKIWSRLIKIWTQGLRKGAQAAKR

SEQ ID NO: 20 - protein, 30 aa, synthetic construct
  REGION 1..30: note = FSD127
HHHHHHLLKIWSRLIKGWTQGWRTIAQALG

SEQ ID NO: 21 - protein, 31 aa, synthetic construct
  REGION 1..31: note = FSD129
FLKIWSRLIKIWTQGGSGGGSRRKGAQAAFR

SEQ ID NO: 22 - protein, 37 aa, synthetic construct
  REGION 1..37: note = FSD131
HHHHHHFLKIWSRLIKIWTQGRRKGAQAAFRHHHHHH

SEQ ID NO: 23 - protein, 30 aa, synthetic construct
  REGION 1..30: note = FSD134
LIRKWIHLIHSWFQNLRRLYARAAARQARA

SEQ ID NO: 24 - protein, 32 aa, synthetic construct
  REGION 1..32: note = FSD146
LLKLWSRLLKLWTQGGSPPPSGRRLKAKRAKA

SEQ ID NO: 25 - protein, 32 aa, synthetic construct
  REGION 1..32: note = FSD155
LLKLWSRLLKLWTQGGSGEGSGRRLKAKRAKA

SEQ ID NO: 26 - protein, 23 aa, synthetic construct
  REGION 1..23: note = FSD156
WIRLFTKLWRIFQQGKRIKAKRA

SEQ ID NO: 27 - protein, 29 aa, synthetic construct
  REGION 1..29: note = FSD157
WIRLFTKLWRIFQQGGSGGGSKRIKAKRA

SEQ ID NO: 28 - protein, 29 aa, synthetic construct
  REGION 1..29: note = FSD159
WIRLFTKLWRIFRQGGSGGGSKRIKAKAA

SEQ ID NO: 29 - protein, 25 aa, synthetic construct
  REGION 1..25: note = FSD162
ILKLWSRLIKIWTQGRRKKAQAAKR

SEQ ID NO: 30 - protein, 25 aa, synthetic construct
  REGION 1..25: note = FSD168
LLKIWSRLIKIWTQGRRLGARAQAR

SEQ ID NO: 31 - protein, 27 aa, synthetic construct
  REGION 1..27: note = FSD173
LLKIWSRLIKIWTQGRRLGARAARQAR

SEQ ID NO: 32 - protein, 33 aa, synthetic construct
  REGION 1..33: note = FSD174
LLKIWSRLIKIWTQGRRLGGSGGGSARAARQAR

SEQ ID NO: 33 - protein, 32 aa, synthetic construct
  REGION 1..32: note = FSD194
KWKLLKLWSRLLKLWGGSGGGSRRLKAKRAKA

SEQ ID NO: 34 - protein, 23 aa, synthetic construct
  REGION 1..23: note = FSD220
WARAFAKAWRIFQQGKRIKAKRA

SEQ ID NO: 35 - protein, 30 aa, synthetic construct
  REGION 1..30: note = FSD250
KWKLLKLWSRLLKLWGGSGGGSARAARQAR

SEQ ID NO: 36 - protein, 30 aa, synthetic construct; identical to SEQ ID NO: 35
  REGION 1..30: note = FSD250D
  SITE 1..30: note = D-amino acid

SEQ ID NO: 37 - protein, 32 aa, synthetic construct
  REGION 1..32: note = FSD253
KWKLLKLWSRLLKLWGGSRGGRGSARAARQAR

SEQ ID NO: 38 - protein, 29 aa, synthetic construct
  REGION 1..29: note = FSD258
WKLLKLWSRLLKLWGGSGGGSARAARQAR

SEQ ID NO: 39 - protein, 30 aa, synthetic construct
  REGION 1..30: note = FSD262
KWKLLRLWSRLLRLWGGSGGGSARAARQAR

SEQ ID NO: 40 - protein, 31 aa, synthetic construct
  REGION 1..31: note = FSD263
KWKLLKLWSRLLKLWGGSGGGSARAAARQAR

SEQ ID NO: 41 - protein, 31 aa, synthetic construct
  REGION 1..31: note = FSD264
KWKLLKLWSRLLKLWGGSGGGSARAAARAAR

SEQ ID NO: 42 - protein, 32 aa, synthetic construct
  REGION 1..32: note = FSD265
KWKLLKLWSRLLKLWGGSGGGSQARAAARQAR

SEQ ID NO: 43 - protein, 32 aa, synthetic construct
  REGION 1..32: note = FSD268
KWKLLKLWSRLLKLWGGSGGGSQARAQARQAR

SEQ ID NO: 44 - protein, 33 aa, synthetic construct
  REGION 1..33: note = FSD286
KWKLLRALARLLKLAGGSGGGSQRRLGARAQAR

SEQ ID NO: 45 - protein, 31 aa, synthetic construct
  REGION 1..31: note = FSD271
KWKLLKLWSRLLKLWRGGSGGGSARAARQAR

SEQ ID NO: 46 - protein, 30 aa, synthetic construct
  REGION 1..30: note = FSD272
KWKLAKLWSRLLKLWGGSGGGSARAARQAR

SEQ ID NO: 47 - protein, 30 aa, synthetic construct
  REGION 1..30: note = FSD273
KWKLLRAWSRLLKLWGGSGGGSARAARQAR

SEQ ID NO: 48 - protein, 30 aa, synthetic construct
  REGION 1..30: note = FSD276
KWKLLKLWSRLLRAWGGSGGGSARAARQAR

SEQ ID NO: 49 - protein, 32 aa, synthetic construct; identical to SEQ ID NO: 43
  REGION 1..32: note = FSD268 Cyclic Amide
  SITE 1: note = Cyclic peptide: covalent link between K1 and R32
  SITE 32: note = Cyclic peptide: covalent link between K1 and R32

SEQ ID NO: 50 - protein, 34 aa, synthetic construct
  REGION 1..34: note = FSD268 Disulfide
  SITE 1: note = Cyclic peptide: disulfide bond between C1 and C34
  SITE 34: note = Cyclic peptide: disulfiude bond between C1 and C34
CKWKLLKLWSRLLKLWGGSGGGSQARAQARQARC

SEQ ID NO: 51 - protein, 34 aa, synthetic construct; identical to SEQ ID NO: 13
  REGION 1..34: note = FSD10 Scarmble

SEQ ID NO: 52 - protein, 32 aa, synthetic construct; identical to SEQ ID NO: 43
  REGION 1..32: note = FSD268 Scramble

SEQ ID NO: 53 - protein, 33 aa, synthetic construct
  REGION 1..33: note = FSD174 Scramble
LGRSGRIKIGGWSALASRARQARGLKIWTQGRL

SEQ ID NO: 54 - protein, 36 aa, synthetic construct
  REGION 1..36: note = FSN3
HHHHHHQFLCFWLNKMGKHNTVWHGRHLKCHKRGKG

SEQ ID NO: 55 - protein, 35 aa, synthetic construct
  REGION 1..35: note = FSN4
HHHHHHLLYLWRRLLKFWCAGRRVYAKCAKAYGCF

SEQ ID NO: 56 - protein, 31 aa, synthetic construct
  REGION 1..31: note = FSN7
LIKLWSRFIKFWTQGRRIKAKLARAGQSWFG

SEQ ID NO: 57 - protein, 19 aa, synthetic construct
  REGION 1..19: note = FSN8
HHHHHHFRKLWLAIVRAKK

SEQ ID NO: 58 - protein, 31 aa, synthetic construct
  REGION 1..31: note = FSD117
HHHHHHFLKFWSRLFKFWTQGRRKGAQAAFR

SEQ ID NO: 59 - protein, 31 aa, synthetic construct
  REGION 1..31: note = FSD118
HHHHHHILKIWSRLIKIWTQGRRKGAQAAIR

SEQ ID NO: 60 - protein, 32 aa, synthetic construct
  REGION 1..32: note = FSD119
HHHHHHFLKIWSRALIKIWTQGLRKGAQAAKR

SEQ ID NO: 61 - protein, 31 aa, synthetic construct
  REGION 1..31: note = FSD121
HHHHHHVLKIWSRLIKIWTQGRRKGAQAAVR

SEQ ID NO: 62 - protein, 31 aa, synthetic construct
  REGION 1..31: note = FSD122
HHHHHHFLKVWSRLVKVWTQGRRKGAQAAFR

SEQ ID NO: 63 - protein, 31 aa, synthetic construct
  REGION 1..31: note = FSD123
HHHHHHVLKVWSRLVKVWTQGRRKGAQAAVR

SEQ ID NO: 64 - protein, 31 aa, synthetic construct
  REGION 1..31: note = FSD124
HHHHHHFLKIWQRLIKIWQQGRRKGAQAAFR

SEQ ID NO: 65 - protein, 31 aa, synthetic construct
  REGION 1..31: note = FSD125
HHHHHHFLKIWNRLIKIWNNGRRKGANAAFR

SEQ ID NO: 66 - protein, 30 aa, synthetic construct
  REGION 1..30: note = FSD126
HHHHHHFLKIWSRLIKIWTQGWRTGAQAGF

SEQ ID NO: 67 - protein, 30 aa, synthetic construct; identical to SEQ ID NO: 20
  REGION 1..30: note = FSD127

SEQ ID NO: 68 - protein, 31 aa, synthetic construct
  REGION 1..31: note = FSD128
HHHHHHFLKIWSRLIKIWPQPRRKGAQAAFR

SEQ ID NO: 69 - protein, 31 aa, synthetic construct
  REGION 1..31: note = FSD130
LIKIWTQFLKIWSRGGSGGGSRRLGARAQAR

SEQ ID NO: 70 - protein, 31 aa, synthetic construct
  REGION 1..31: note = FSD132
HHHHHHRFAAQAGKRRGQTWIKILRSWIKLF

SEQ ID NO: 71 - protein, 31 aa, synthetic construct
  REGION 1..31: note = FSD133
HHHHFLHHSWIKKILRTWIRRGQQAGKFAAR

SEQ ID NO: 72 - protein, 29 aa, synthetic construct
  REGION 1..29: note = FSD135
LIRKWIHLIHSWFQNLRRLQARAQARQAR

SEQ ID NO: 73 - protein, 29 aa, synthetic construct
  REGION 1..29: note = FSD137
LLRKWSHLLHIWGGSGGGSQARAQARQAR

SEQ ID NO: 74 - protein, 33 aa, synthetic construct
  REGION 1..33: note = FSD138
KWKLARAFARAIKIFGGSGGGSQRRLKAKRAKA

SEQ ID NO: 75 - protein, 40 aa, synthetic construct
  REGION 1..40: note = FSD139
HHHHHHLIRLWSHLIHIWFQNRRLKWKKKYARAAARQARA

SEQ ID NO: 76 - protein, 46 aa, synthetic construct
  REGION 1..46: note = FSD140
HHHHHHLIRLWSHLIHIWFQNRRLKWKKKYARAAARQARAHHHHHH

SEQ ID NO: 77 - protein, 41 aa, synthetic construct
  REGION 1..41: note = FSD141
LIRLWSHLIHIWFQNRRLKWKKKGGSGGGSYARAAARQARA

SEQ ID NO: 78 - protein, 25 aa, synthetic construct
  REGION 1..25: note = FSD142
FLKIWSHLIHIWTQGRRLKAKRAKA

SEQ ID NO: 79 - protein, 22 aa, synthetic construct
  REGION 1..22: note = FSD143
LIRKWIHLIHSWFQGRRLGARA

SEQ ID NO: 80 - protein, 49 aa, synthetic construct
  REGION 1..49: note = FSD144
HHHHHHKKALLAHALHLLALLALHLAHALKKAYGRKKRRQRRRHHHHHH

SEQ ID NO: 81 - protein, 52 aa, synthetic construct
  REGION 1..52: note = FSD145
HHHHHHKKHLLAHALHLLALLALHLAHALAHLKKAYGRKKRRQRRRHHHHHH

SEQ ID NO: 82 - protein, 31 aa, synthetic construct
  REGION 1..31: note = FSD147
LLKLWTQLLKLWSRGGSGGGSRRLKAKRAKA

SEQ ID NO: 83 - protein, 28 aa, synthetic construct
  REGION 1..28: note = FSD148
HHHHHHMVTVLFRRLRIRRACGPPRVRV

SEQ ID NO: 84 - protein, 28 aa, synthetic construct
  REGION 1..28: note = FSD149
HHHHHHMVRVLTRFLRIGARCRRPPVVR

SEQ ID NO: 85 - protein, 30 aa, synthetic construct
  REGION 1..30: note = FSD150
HHHHHHWITWLFKRLKIRRAAGQSKFRIAG

SEQ ID NO: 86 - protein, 30 aa, synthetic construct
  REGION 1..30: note = FSD151
HHHHHHWITWLRKILKRFRKAAQSGFRIAG

SEQ ID NO: 87 - protein, 30 aa, synthetic construct
  REGION 1..30: note = FSD152
HHHHHHWITWLRKILKRFGKAAQSGFRIAR

SEQ ID NO: 88 - protein, 30 aa, synthetic construct
  REGION 1..30: note = FSD153
HHHHHHWITWLRKILKRLGGAAQSIITGGQ

SEQ ID NO: 89 - protein, 36 aa, synthetic construct
  REGION 1..36: note = FSD154
HHHHHHWITWLFKRLKIRRAAGGSGGGSQSKFRIAG

SEQ ID NO: 90 - protein, 23 aa, synthetic construct
  REGION 1..23: note = FSD158
WIRLFTKLWRIFRQGKRIKAKAA

SEQ ID NO: 91 - protein, 25 aa, synthetic construct
  REGION 1..25: note = FSD160
ILKLWSRLIKIWTQGRRLGAQAALR

SEQ ID NO: 92 - protein, 31 aa, synthetic construct
  REGION 1..31: note = FSD161
ILKLWSRLIKIWTQGGSGGGSRRLGAQAALR

SEQ ID NO: 93 - protein, 31 aa, synthetic construct
  REGION 1..31: note = FSD163
ILKLWSRLIKIWTQGGSGGGSRRKKAQAAKR

SEQ ID NO: 94 - protein, 25 aa, synthetic construct
  REGION 1..25: note = FSD164
LLKIWSRLIKIWTQGRRLGARAARA

SEQ ID NO: 95 - protein, 31 aa, synthetic construct
  REGION 1..31: note = FSD165
LLKIWSRLIKIWTQGGSGGGSRRKKARAARA

SEQ ID NO: 96 - protein, 26 aa, synthetic construct
  REGION 1..26: note = FSD166
LLKLWSRLIKIWTKGRRKKARAAQAR

SEQ ID NO: 97 - protein, 32 aa, synthetic construct
  REGION 1..32: note = FSD167
LLKLWSRLIKIWTKGGSGGGSRRKKARAAQAR

SEQ ID NO: 98 - protein, 31 aa, synthetic construct
  REGION 1..31: note = FSD169
LLKIWSRLIKIWTQGGSGGGSRRLGARAQAR

SEQ ID NO: 99 - protein, 25 aa, synthetic construct
  REGION 1..25: note = FSD170
LIKIWTQLLKIWSRGRRLGARAQAR

SEQ ID NO: 100 - protein, 25 aa, synthetic construct
  REGION 1..25: note = FSD171
  SITE 1: note = Acetyl
  SITE 25: note = Amide
  SITE 25: note = Cysteamide
LLKIWSRLIKIWTQGRRLGARAQAR

SEQ ID NO: 101 - protein, 31 aa, synthetic construct
  REGION 1..31: note = FSD172
LLKIWSRLIKIWTQGRRLGGSGGGSARAQAR

SEQ ID NO: 102 - protein, 29 aa, synthetic construct
  REGION 1..29: note = FSD175
LLKIWSRLIKIWTQGRRLGGSARAARQAR

SEQ ID NO: 103 - protein, 36 aa, synthetic construct
  REGION 1..36: note = FSD176
LLKIWSRLIKIWTQGRRLGGSGGGSGGSARAARQAR

SEQ ID NO: 104 - protein, 25 aa, synthetic construct
  REGION 1..25: note = FSD177
KLKIWSRLIRKWTKGLRLGAQAQAR

SEQ ID NO: 105 - protein, 31 aa, synthetic construct
  REGION 1..31: note = FSD178
KLKIWSRLIRKWTKGGSGGGSLRLGAQAQAR

SEQ ID NO: 106 - protein, 28 aa, synthetic construct
  REGION 1..28: note = FSD179
LLKIWSRLIKIWTQGRGRESRKPRKSRQ

SEQ ID NO: 107 - protein, 34 aa, synthetic construct
  REGION 1..34: note = FSD180
LLKIWSRLIKIWTQGGSGGGSRGRESRKPRKSRQ

SEQ ID NO: 108 - protein, 28 aa, synthetic construct
  REGION 1..28: note = FSD181
LLKIWSRLIKIWTQGLGLLVLRVRAGKR

SEQ ID NO: 109 - protein, 34 aa, synthetic construct
  REGION 1..34: note = FSD182
LLKIWSRLIKIWTQGGSGGGSLGLLVLRVRAGKR

SEQ ID NO: 110 - protein, 22 aa, synthetic construct
  REGION 1..22: note = FSD183
LLKIWSRLIKIWTQGRRLGARA

SEQ ID NO: 111 - protein, 24 aa, synthetic construct
  REGION 1..24: note = FSD184
LLKIWSRLIKIWTQGRRLGARAAR

SEQ ID NO: 112 - protein, 25 aa, synthetic construct
  REGION 1..25: note = FSD185
LLKIWSRLIKIWTQGRRLGARAARQ

SEQ ID NO: 113 - protein, 27 aa, synthetic construct
  REGION 1..27: note = FSD186
LLKIWSRLIKIWTQGRGLEARAPRKAR

SEQ ID NO: 114 - protein, 28 aa, synthetic construct
  REGION 1..28: note = FSD187
LLKIWSRLIKIWTQGRRLGARKPRKSRQ

SEQ ID NO: 115 - protein, 27 aa, synthetic construct
  REGION 1..27: note = FSD188
LLKIWSRLIKIWTQGRGRESRAARQAR

SEQ ID NO: 116 - protein, 27 aa, synthetic construct
  REGION 1..27: note = FSD189
LLKIWSRLIKIWTQGRRLGRAQRAQRA

SEQ ID NO: 117 - protein, 23 aa, synthetic construct
  REGION 1..23: note = FSD190
LLKIWSRLIKIWTQGRAQRAQRA

SEQ ID NO: 118 - protein, 32 aa, synthetic construct
  REGION 1..32: note = FSD191
HHHHHHLLKIWSRLIKIWTQGTRSKRAGLQFP

SEQ ID NO: 119 - protein, 31 aa, synthetic construct
  REGION 1..31: note = FSD192
HHHHHHLLKIWSRLIKIWTQGVGRVHRLLRK

SEQ ID NO: 120 - protein, 33 aa, synthetic construct
  REGION 1..33: note = FSD193
KWKLLKIWSRLIKIWRRLGGSGGGSARAARQAR

SEQ ID NO: 121 - protein, 25 aa, synthetic construct
  REGION 1..25: note = FSD195
LLKIWSRLIKIWTQGRRLKARAQAR

SEQ ID NO: 122 - protein, 25 aa, synthetic construct
  REGION 1..25: note = FSD196
LLKIWSRLIKIWTQGRRLGARAAAR

SEQ ID NO: 123 - protein, 25 aa, synthetic construct
  REGION 1..25: note = FSD197
LLKIWSRLIKIWTQGRRLKARAAAR

SEQ ID NO: 124 - protein, 27 aa, synthetic construct
  REGION 1..27: note = FSD198
WSRLIKIWTQGGSGGGSRRKGAQAAFR

SEQ ID NO: 125 - protein, 23 aa, synthetic construct
  REGION 1..23: note = FSD199
WSRLITKIWRIFTQGRRLGARAA

SEQ ID NO: 126 - protein, 23 aa, synthetic construct
  REGION 1..23: note = FSD200
WSRLITKIWRIFTQGRRLKARAA

SEQ ID NO: 127 - protein, 19 aa, synthetic construct
  REGION 1..19: note = FSD201
WSRLIKLWTQGRRLKARAA

SEQ ID NO: 128 - protein, 19 aa, synthetic construct
  REGION 1..19: note = FSD202
WIRLFKLWQQGKRIKAKRA

SEQ ID NO: 129 - protein, 21 aa, synthetic construct
  REGION 1..21: note = FSD203
WSRLIKIWTQGRRLGARAQAR

SEQ ID NO: 130 - protein, 21 aa, synthetic construct
  REGION 1..21: note = FSD204
LLKIWSRLIKIWTQGRRLGAR

SEQ ID NO: 131 - protein, 17 aa, synthetic construct
  REGION 1..17: note = FSD205
WSRLIKIWTQGRRLGAR

SEQ ID NO: 132 - protein, 21 aa, synthetic construct
  REGION 1..21: note = FSD206
KIWSRLIKIWTQGRRLGARAQ

SEQ ID NO: 133 - protein, 25 aa, synthetic construct
  REGION 1..25: note = FSD207
LAKAWARAIKIWTQGRRLGARAQAR

SEQ ID NO: 134 - protein, 32 aa, synthetic construct
  REGION 1..32: note = FSD208
KWKLARAFARAIKKLGGSGGGSRRLGARAQAR

SEQ ID NO: 135 - protein, 33 aa, synthetic construct
  REGION 1..33: note = FSD209
LLKIWSRLIKIWTQGGSGGGSYARALRRQARTG

SEQ ID NO: 136 - protein, 32 aa, synthetic construct
  REGION 1..32: note = FSD210
KWKLARAFARAIKKLGGSGGGSRRLKAKRAKA

SEQ ID NO: 137 - protein, 34 aa, synthetic construct
  REGION 1..34: note = FSD211
KWKLLKLWSRLLKLWGGSGGGSYARALRRQARTG

SEQ ID NO: 138 - protein, 25 aa, synthetic construct
  REGION 1..25: note = FSD212
WSRLLKLWGGSGGGSRRLKAKRAKA

SEQ ID NO: 139 - protein, 25 aa, synthetic construct
  REGION 1..25: note = FSD213
LLKLWSRLLKLWGGSGGGSRRLKAK

SEQ ID NO: 140 - protein, 21 aa, synthetic construct
  REGION 1..21: note = FSD214
WSRLLKLWGGSGGGSRRLKAK

SEQ ID NO: 141 - protein, 25 aa, synthetic construct
  REGION 1..25: note = FSD215
KLWSRLLKLWGGSGGGSRRLKAKRA

SEQ ID NO: 142 - protein, 27 aa, synthetic construct
  REGION 1..27: note = FSD216
LLKIWSRLIKIWTQGRGRSRKPRKSRQ

SEQ ID NO: 143 - protein, 22 aa, synthetic construct
  REGION 1..22: note = FSD217
KWKLKLWRLKGGSGGGSRRAKA

SEQ ID NO: 144 - protein, 31 aa, synthetic construct
  REGION 1..31: note = FSD218
KWKLKLWRLKSRLKLWRLKGGSGGGSRRAKA

SEQ ID NO: 145 - protein, 23 aa, synthetic construct
  REGION 1..23: note = FSD219
WIRLWTHLWHIWQQGKRIKAKRA

SEQ ID NO: 146 - protein, 24 aa, synthetic construct
  REGION 1..24: note = FSD221
WKLIRLFTRLIKIWGQRRLKAKRA

SEQ ID NO: 147 - protein, 22 aa, synthetic construct
  REGION 1..22: note = FSD222
LARAFARAIKIFGQRRLKAKRA

SEQ ID NO: 148 - protein, 28 aa, synthetic construct
  REGION 1..28: note = FSD223
LARAFARAIKIFQGGSGGGSRRLKAKRA

SEQ ID NO: 149 - protein, 25 aa, synthetic construct
  REGION 1..25: note = FSD224
KWKLARAFARAIKIFGQRRLKAKRA

SEQ ID NO: 150 - protein, 31 aa, synthetic construct
  REGION 1..31: note = FSD225
KWKLARAFARAIKIFGGSGGGSQRRLKAKRA

SEQ ID NO: 151 - protein, 27 aa, synthetic construct
  REGION 1..27: note = FSD226
KWKLARAFARAIKIFGQRRLGARAQAR

SEQ ID NO: 152 - protein, 33 aa, synthetic construct
  REGION 1..33: note = FSD227
KWKLARAFARAIKIFGGSGGGSQRRLGARAQAR

SEQ ID NO: 153 - protein, 27 aa, synthetic construct
  REGION 1..27: note = FSD228
KWKLARAFARAIKIFGQRRLKAKRAKA

SEQ ID NO: 154 - protein, 33 aa, synthetic construct; identical to SEQ ID NO: 74
  REGION 1..33: note = FSD229

SEQ ID NO: 155 - protein, 26 aa, synthetic construct
  REGION 1..26: note = FSD230
KWKLAKAWARALKLWGRRLGARAQAR

SEQ ID NO: 156 - protein, 33 aa, synthetic construct
  REGION 1..33: note = FSD231
KWKLARAFARAIKKLGGSGGGSRRLKAKRALKA

SEQ ID NO: 157 - protein, 32 aa, synthetic construct
  REGION 1..32: note = FSD232
KWKWARAWARAWKKWGGSGGGSRRLGARAQAR

SEQ ID NO: 158 - protein, 32 aa, synthetic construct
  REGION 1..32: note = FSD233
KLKLARALARALKKLGGSGGGSRRLGARAQAR

SEQ ID NO: 159 - protein, 32 aa, synthetic construct
  REGION 1..32: note = FSD234
KIKIARAIARAIKKIGGSGGGSRRLGARAQAR

SEQ ID NO: 160 - protein, 32 aa, synthetic construct
  REGION 1..32: note = FSD235
KFKFARAFARAFKKFGGSGGGSRRLGARAQAR

SEQ ID NO: 161 - protein, 39 aa, synthetic construct
  REGION 1..39: note = FSD236
KWKLLKLWSRLLKLWSRLLKLWGGSGGGSRRLGARAQAR

SEQ ID NO: 162 - protein, 39 aa, synthetic construct
  REGION 1..39: note = FSD237
KWKLLKLWTQLLKLWTQLLKLWGGSGGGSRRLGARAQAR

SEQ ID NO: 163 - protein, 32 aa, synthetic construct
  REGION 1..32: note = FSD238
KWKLLKIWSRLIKIWGGSGGGSQARAQARQAR

SEQ ID NO: 164 - protein, 31 aa, synthetic construct
  REGION 1..31: note = FSD239
KWKLLKIWTQLIKIWGGSGGGSQARQARQAR

SEQ ID NO: 165 - protein, 33 aa, synthetic construct
  REGION 1..33: note = FSD240
KWKALLALALHLAHLALHLKKAGRRKGAQAAFR

SEQ ID NO: 166 - protein, 33 aa, synthetic construct
  REGION 1..33: note = FSD241
KWKLARAFARAIKKLGGSGGGSYARAAARQARA

SEQ ID NO: 167 - protein, 36 aa, synthetic construct
  REGION 1..36: note = FSD243
LLKIWSRLIKIWTQGRRLGGSGGGSYARAAARQARA

SEQ ID NO: 168 - protein, 34 aa, synthetic construct
  REGION 1..34: note = FSD244
KWKLAKAWARALKLWGGSGGGSYARAAARKAKRA

SEQ ID NO: 169 - protein, 34 aa, synthetic construct
  REGION 1..34: note = FSD246
KWKLLKLWSRLLKLWGGSGGGSYARAAARKAKRA

SEQ ID NO: 170 - protein, 37 aa, synthetic construct
  REGION 1..37: note = FSD247
LLKIWSRLIKIWTQGRRLGGSGGGSYARAAARKAKRA

SEQ ID NO: 171 - protein, 30 aa, synthetic construct
  REGION 1..30: note = FSD248
KWKLAKAWARALKLWGGSGGGSARAARQAR

SEQ ID NO: 172 - protein, 30 aa, synthetic construct
  REGION 1..30: note = FSD250 Scramble
RGKLWSLSKLKGWGGARASKAQLARLGLWR

SEQ ID NO: 173 - protein, 30 aa, synthetic construct
  REGION 1..30: note = FSD250E
KWKLLELWSELLELWGGSGGGSARAARQAR

SEQ ID NO: 174 - protein, 30 aa, synthetic construct
  REGION 1..30: note = FSD251
KWKLLKLWSRLLKLWGGSGGGSAEAAEQAE

SEQ ID NO: 175 - protein, 32 aa, synthetic construct; identical to SEQ ID NO: 37
  REGION 1..32: note = FSD254

SEQ ID NO: 176 - protein, 32 aa, synthetic construct
  REGION 1..32: note = FSD255
KWKLLKLWGGSRLLKLWGGSGGGSARAARQAR

SEQ ID NO: 177 - protein, 33 aa, synthetic construct
  REGION 1..33: note = FSD256
KWKLLKLGRWSRLGLKLWGGSGGGSARAARQAR

SEQ ID NO: 178 - protein, 32 aa, synthetic construct
  REGION 1..32: note = FSD257
KWKLLKLWAASRLLKLWGGSGGGSARAARQAR

SEQ ID NO: 179 - protein, 33 aa, synthetic construct
  REGION 1..33: note = FSD259
KWKLLKLARWSRLALKLWGGSGGGSARAARQAR

SEQ ID NO: 180 - protein, 30 aa, synthetic construct
  REGION 1..30: note = FSD260
RWRLLRLWSRLLRLWGGSGGGSARAARQAR

SEQ ID NO: 181 - protein, 30 aa, synthetic construct
  REGION 1..30: note = FSD261
GGSLLKLWSRLLKLWGGSGGGSARAARQAR

SEQ ID NO: 182 - protein, 27 aa, synthetic construct
  REGION 1..27: note = FSD266
LLKLWSRLLKLWGGSGGGSARAARQAR

SEQ ID NO: 183 - protein, 31 aa, synthetic construct
  REGION 1..31: note = FSD267
KWKLLKLWSRLLKLWGGSGGGSYARAARYAR

SEQ ID NO: 184 - protein, 32 aa, synthetic construct
  REGION 1..32: note = FSD269
KWKLLKLWSRLLKLWGGSGGGSYARAYARYAR

SEQ ID NO: 185 - protein, 28 aa, synthetic construct
  REGION 1..28: note = FSD270
KWKLLKLWSRLLKLWGGSGGGSAAAAEK

SEQ ID NO: 186 - protein, 30 aa, synthetic construct
  REGION 1..30: note = FSD274
KWKLARAWSRLLKLWGGSGGGSARAARQAR

SEQ ID NO: 187 - protein, 30 aa, synthetic construct
  REGION 1..30: note = FSD275
KWKLLKLWSRLAKLWGGSGGGSARAARQAR

SEQ ID NO: 188 - protein, 30 aa, synthetic construct; identical to SEQ ID NO: 48
  REGION 1..30: note = FSD276

SEQ ID NO: 189 - protein, 30 aa, synthetic construct
  REGION 1..30: note = FSD277
KWKLLKLWSRLARAWGGSGGGSARAARQAR

SEQ ID NO: 190 - protein, 30 aa, synthetic construct
  REGION 1..30: note = FSD278
KWKLARAWSRLARAWGGSGGGSARAARQAR

SEQ ID NO: 191 - protein, 29 aa, synthetic construct
  REGION 1..29: note = FSD279
KWKLARALARAWSRGGSGGGSARAARQAR

SEQ ID NO: 192 - protein, 30 aa, synthetic construct
  REGION 1..30: note = FSD280
KWKLLKLWKRLLKKWGGSGGGSARAARQAR

SEQ ID NO: 193 - protein, 30 aa, synthetic construct
  REGION 1..30: note = FSD281
KWSLLKLWSALLKLWGGSGGGSARAARQAR

SEQ ID NO: 194 - protein, 30 aa, synthetic construct
  REGION 1..30: note = FSD282
KWKLWKLLSRLWKLLGGSGGGSARAARQAR

SEQ ID NO: 195 - protein, 30 aa, synthetic construct
  REGION 1..30: note = FSD283
KWKLARKFKRAIKKFGGSGGGSARAARQAR

SEQ ID NO: 196 - protein, 30 aa, synthetic construct
  REGION 1..30: note = FSD284
KWALARAFARAIAIFGGSGGGSARAARQAR

SEQ ID NO: 197 - protein, 30 aa, synthetic construct
  REGION 1..30: note = FSD285
LARAFARAIKIFGGSGGGSQRRLGARAQAR

SEQ ID NO: 198 - protein, 33 aa, synthetic construct
  REGION 1..33: note = FSD287
KWKLLRALARLLKALGGSGGGSQRRLGARAQAR

SEQ ID NO: 199 - protein, 32 aa, synthetic construct
  REGION 1..32: note = FSD288
KWKLLKWWSRLLKWWGGSGGGSQARAQARQAR

SEQ ID NO: 200 - protein, 32 aa, synthetic construct
  REGION 1..32: note = FSD289
KWKLLKFWSRLLKFWGGSGGGSQARAQARQAR

SEQ ID NO: 201 - protein, 32 aa, synthetic construct
  REGION 1..32: note = FSD290
KWKLLKLYSRLLKLYGGSGGGSQARAQARQAR

SEQ ID NO: 202 - protein, 32 aa, synthetic construct
  REGION 1..32: note = FSD291
KWKLLKLFSRLLKLFGGSGGGSQARAQARQAR

SEQ ID NO: 203 - protein, 32 aa, synthetic construct
  REGION 1..32: note = FSD292
KWKLLSLWSSLLSLWGGSGGGSQARAQARQAR

SEQ ID NO: 204 - protein, 32 aa, synthetic construct
  REGION 1..32: note = FSD293
KWKLLSLWSRLLSLWGGSGGGSQARAQARQAR

SEQ ID NO: 205 - protein, 32 aa, synthetic construct
  REGION 1..32: note = FSD2294
KWKLLKLWSSLLKLWGGSGGGSQARAQARQAR

SEQ ID NO: 206 - protein, 31 aa, synthetic construct
  REGION 1..31: note = FSD295
KWKLLKLWSLLKLWGGSGGGSQARAQARQAR

SEQ ID NO: 207 - protein, 34 aa, synthetic construct
  REGION 1..34: note = FSD296
KWKLLKLWSRLLKLWQQGGSGGGSQARAQARQAR

SEQ ID NO: 208 - protein, 34 aa, synthetic construct
  REGION 1..34: note = FSD297
KWKLLKLWSRLLKLWNNGGSGGGSQARAQARQAR

SEQ ID NO: 209 - protein, 32 aa, synthetic construct
  REGION 1..32: note = FSD298
SWSLLKLWSRLLKLWGGSGGGSQARAQARQAR

SEQ ID NO: 210 - protein, 30 aa, synthetic construct
  REGION 1..30: note = FSD299
KWKLLKLWSRLLKLWIKIFGQARAQARQAR

SEQ ID NO: 211 - protein, 30 aa, synthetic construct
  REGION 1..30: note = FSD300
KWKLLKLWSRLLKLWWRIFGQARAQARQAR

SEQ ID NO: 212 - protein, 29 aa, synthetic construct
  REGION 1..29: note = FSD301
GGSGGGSKWKLLKLWSRLLKLWGGSGGGS

SEQ ID NO: 213 - protein, 28 aa, synthetic construct
  REGION 1..28: note = FSD302
KWKLLKLWSRLLKLWGGGQARAQARQAR

SEQ ID NO: 214 - protein, 26 aa, synthetic construct
  REGION 1..26: note = FSD303
KWKLLKLWSRLLKLWGQARAQARQAR

SEQ ID NO: 215 - protein, 25 aa, synthetic construct
  REGION 1..25: note = FSD304
KWKLLKLWSRLLKLWQARAQARQAR

SEQ ID NO: 216 - protein, 32 aa, synthetic construct
  REGION 1..32: note = FSD305
KWKLLKLWSRLLKLWGGGGGGGQARAQARQAR

SEQ ID NO: 217 - protein, 28 aa, synthetic construct
  REGION 1..28: note = FSD306
KWKLLKLWSRLLKLWGGSGGGSQARQAR

SEQ ID NO: 218 - protein, 25 aa, synthetic construct
  REGION 1..25: note = FSD307
KWKLLKLWSRLLKLWGGSGGGSQAR

SEQ ID NO: 219 - protein, 32 aa, synthetic construct
  REGION 1..32: note = FSD308
KWKLLKLWSRLLKLWGGSGGGSGARAGARGAR

SEQ ID NO: 220 - protein, 32 aa, synthetic construct
  REGION 1..32: note = FSD309
KWKLLKLWSRLLKLWGGSGGGSQAGAQAGQAG

SEQ ID NO: 221 - protein, 32 aa, synthetic construct
  REGION 1..32: note = FSD310
KWKLLKLWSRLLKLWGGSGGGSQGRGQGRQGR

SEQ ID NO: 222 - protein, 30 aa, synthetic construct
  REGION 1..30: note = FSD311
KWKLLKLWSRLLKLWGGSGGGSRGGRGGGR

SEQ ID NO: 223 - protein, 28 aa, synthetic construct
  REGION 1..28: note = FSD312
WIRLFTKLWIFQQGGSGGGSKRIKAKRA

SEQ ID NO: 224 - protein, 29 aa, synthetic construct
  REGION 1..29: note = FSD313
WIRLFSRLWRIFQQGGSGGGSKRIKAKRA

SEQ ID NO: 225 - protein, 32 aa, synthetic construct
  REGION 1..32: note = FSD314
KWKWIRLFSRLWRIFQQGGSGGGSKRIKAKRA

SEQ ID NO: 226 - protein, 31 aa, synthetic construct
  REGION 1..31: note = FSD315
WIRLFSRLWRIFQQGGSGGGSQARAQARQAR

SEQ ID NO: 227 - protein, 34 aa, synthetic construct
  REGION 1..34: note = FSD316
KWKWIRLFSRLWRIFQQGGSGGGSQARAQARQAR

SEQ ID NO: 228 - protein, 30 aa, synthetic construct
  REGION 1..30: note = FSD317
WIRLFTKLWQIFQQGGGSGGGSARAARQAR

SEQ ID NO: 229 - protein, 30 aa, synthetic construct
  REGION 1..30: note = FSD318
WIRLFTKLWRIFQQGGGSGGGSARAARQAR

SEQ ID NO: 230 - protein, 28 aa, synthetic construct
  REGION 1..28: note = FSD319
KWKLLKLWSRLLKLWGGSGGGSAAAAQK

SEQ ID NO: 231 - protein, 28 aa, synthetic construct
  REGION 1..28: note = FSD320
KWKLLKLWSRLLKLWGGSGGGSAAAAQQ

SEQ ID NO: 232 - protein, 24 aa, synthetic construct
  REGION 1..24: note = FSD321
KWKLAKAWSRAIKIWGARAQARQA

SEQ ID NO: 233 - protein, 30 aa, synthetic construct
  REGION 1..30: note = FSD322
KWKLAKAWSRALKLWGGSGGGSARAQARQA

SEQ ID NO: 234 - protein, 22 aa, synthetic construct
  REGION 1..22: note = FSD323
WIRLFTRLIKIWGQRRLKAKRA

SEQ ID NO: 235 - protein, 22 aa, synthetic construct
  REGION 1..22: note = FSD324
WARAFARAWRIFQQRRLKAKRA

SEQ ID NO: 236 - protein, 25 aa, synthetic construct
  REGION 1..25: note = FSD325
WARAFARAWRIFQQRRLARAARQAR

SEQ ID NO: 237 - protein, 23 aa, synthetic construct
  REGION 1..23: note = FSD326
LARAFARAIKIFGQARAQARQAR

SEQ ID NO: 238 - protein, 23 aa, synthetic construct
  REGION 1..23: note = FSD327
LARAFARAIKIFGRRLKAKRAKA

SEQ ID NO: 239 - protein, 23 aa, synthetic construct
  REGION 1..23: note = FSD328
LARAFARAIKIFGRRLGARAQAR

SEQ ID NO: 240 - protein, 23 aa, synthetic construct
  REGION 1..23: note = FSD330
LARAFARALLKLWGQRRLKAKRA

SEQ ID NO: 241 - protein, 21 aa, synthetic construct
  REGION 1..21: note = FSD331
KWKLARAFARAGQRRLKAKRA

SEQ ID NO: 242 - protein, 22 aa, synthetic construct
  REGION 1..22: note = FSD332
KWKLARAFARAGRRLGARAQAR

SEQ ID NO: 243 - protein, 32 aa, synthetic construct
  REGION 1..32: note = FSD333
KWKLLRLLLRLLKKLGGSGGGSQARAQARQAR

SEQ ID NO: 244 - protein, 32 aa, synthetic construct
  REGION 1..32: note = FSD334
KWKLLRWLWRLLKKLGGSGGGSQARAQARQAR

SEQ ID NO: 245 - protein, 32 aa, synthetic construct
  REGION 1..32: note = FSD335
KWKLARLLLRALKKLGGSGGGSQARAQARQAR

SEQ ID NO: 246 - protein, 32 aa, synthetic construct
  REGION 1..32: note = FSD336
KWKLLRLFLRLFKKLGGSGGGSQARAQARQAR

SEQ ID NO: 247 - protein, 32 aa, synthetic construct
  REGION 1..32: note = FSD337
KWKLARWLWRALKKLGGSGGGSQARAQARQAR

SEQ ID NO: 248 - protein, 32 aa, synthetic construct
  REGION 1..32: note = FSD338
KWKLLRWFWRLFKKLGGSGGGSQARAQARQAR

SEQ ID NO: 249 - protein, 32 aa, synthetic construct
  REGION 1..32: note = FSD339
KWKLARLFLRAFKKLGGSGGGSQARAQARQAR

SEQ ID NO: 250 - protein, 32 aa, synthetic construct
  REGION 1..32: note = FSD340
KWKLARWFWRAFKKLGGSGGGSQARAQARQAR

SEQ ID NO: 251 - protein, 37 aa, synthetic construct
  REGION 1..37: note = FSD341
LLKIWSRLIKIWTQGRRLGGSGGGSYARALRRQARTG

SEQ ID NO: 252 - protein, 34 aa, synthetic construct
  REGION 1..34: note = FSD342
KWKLARWFWRAFKKLGGSGGGSYARALRRQARTG

SEQ ID NO: 253 - protein, 30 aa, synthetic construct
  REGION 1..30: note = FSD343
KWKLLQLWSRLLQLWGGSGGGSARAARQAR

SEQ ID NO: 254 - protein, 30 aa, synthetic construct
  REGION 1..30: note = FSD344
QWQLLKLWSRLLKLWGGSGGGSARAARQAR

SEQ ID NO: 255 - protein, 30 aa, synthetic construct
  REGION 1..30: note = FSD345
KLKLLKLWSRLLKLWGGSGGGSARAARQAR

SEQ ID NO: 256 - protein, 30 aa, synthetic construct
  REGION 1..30: note = FSD346
KFKLLKLFSRLLKLFGGSGGGSARAARQAR

SEQ ID NO: 257 - protein, 30 aa, synthetic construct
  REGION 1..30: note = FSD347
KWKLLKLLSRLLKLLGGSGGGSARAARQAR

SEQ ID NO: 258 - protein, 30 aa, synthetic construct
  REGION 1..30: note = FSD348
KWKLLKLLSRLLKLLGGGGGGGARAARQAR

SEQ ID NO: 259 - protein, 30 aa, synthetic construct
  REGION 1..30: note = FSD349
KWKWLKLWSRLWKLWGGSGGGSARAARQAR

SEQ ID NO: 260 - protein, 30 aa, synthetic construct
  REGION 1..30: note = FSD350
KWKLLKFWSRLLKFWGGSGGGSARAARQAR

SEQ ID NO: 261 - protein, 30 aa, synthetic construct
  REGION 1..30: note = FSD351
KWKLLKLFSRLFKLWGGSGGGSARAARQAR

SEQ ID NO: 262 - protein, 30 aa, synthetic construct
  REGION 1..30: note = FSD352
KWKLLKLWSRLIKIWGGSGGGSARAARQAR

SEQ ID NO: 263 - protein, 30 aa, synthetic construct
  REGION 1..30: note = FSD353
KWKLLKLQSRLLKLQGGSGGGSARAARQAR

SEQ ID NO: 264 - protein, 25 aa, synthetic construct
  REGION 1..25: note = FSD354
KWKLLKLWSRLLKLWGGSGGGSQGR

SEQ ID NO: 265 - protein, 25 aa, synthetic construct
  REGION 1..25: note = FSD355
KWKLLKLWSRLLKLWGGSGGGSGAR

SEQ ID NO: 266 - protein, 25 aa, synthetic construct
  REGION 1..25: note = FSD356
KWKLLKLWSRLLKLWGGSGGGSQAG

SEQ ID NO: 267 - protein, 25 aa, synthetic construct
  REGION 1..25: note = FSD357
KWKLLKLWSRLLKLWGGSGGGSRRR

SEQ ID NO: 268 - protein, 30 aa, synthetic construct
  REGION 1..30: note = FSD358
KWKLLHLWSRLLHLWGGSGGGSARAARQAR

SEQ ID NO: 269 - protein, 30 aa, synthetic construct
  REGION 1..30: note = FSD359
KWKLLKLWSKLLKLWGGGGGGGAKAAKQAK

SEQ ID NO: 270 - protein, 30 aa, synthetic construct
  REGION 1..30: note = FSD360
RWRLLRLWSRLLRLWGGGGGGGARAARQAR

SEQ ID NO: 271 - protein, 27 aa, synthetic construct
  REGION 1..27: note = FSD361
LLKLWSKLLKLWGGGGGGGAKAAKQAK

SEQ ID NO: 272 - protein, 27 aa, synthetic construct
  REGION 1..27: note = FSD362
LLRLWSRLLRLWGGGGGGGARAARQAR

SEQ ID NO: 273 - protein, 23 aa, synthetic construct
  REGION 1..23: note = FSD363
LLKLWSKLLKLWGGGAKAAKQAK

SEQ ID NO: 274 - protein, 23 aa, synthetic construct
  REGION 1..23: note = FSD364
LLRLWSRLLRLWGGGARAARQAR

SEQ ID NO: 275 - protein, 21 aa, synthetic construct
  REGION 1..21: note = FSD365
KWKLLKLWSRLLKLWGGGQAR

SEQ ID NO: 276 - protein, 18 aa, synthetic construct
  REGION 1..18: note = FSD366
KWKLWSRLLKLWGGGQAR

SEQ ID NO: 277 - protein, 19 aa, synthetic construct
  REGION 1..19: note = FSD367
KWKLLKLWSRLLKGGGQAR

SEQ ID NO: 278 - protein, 33 aa, synthetic construct
  REGION 1..33: note = FSD368
KWKLARAFARASRLLKLWGGSGGGSARAARQAR

SEQ ID NO: 279 - protein, 30 aa, synthetic construct
  REGION 1..30: note = FSD369
KWKLARAFARALKLWGGSGGGSARAARQAR

SEQ ID NO: 280 - protein, 30 aa, synthetic construct
  REGION 1..30: note = FSD370
KWKLLKLWSRLLKKLGGSGGGSARAARQAR

SEQ ID NO: 281 - protein, 32 aa, synthetic construct
  REGION 1..32: note = FSD371
KWKLLKLWSRLLKLWQQGGSGGGSARAARQAR

SEQ ID NO: 282 - protein, 36 aa, synthetic construct
  REGION 1..36: note = FSD372
KWKLARAFARAIKKLNNGGSGGGSYARALRRQARTG

SEQ ID NO: 283 - protein, 26 aa, synthetic construct
  REGION 1..26: note = FSD373
GGSGGGSLLKLWSRLLKLWGGSGGGS

SEQ ID NO: 284 - protein, 32 aa, synthetic construct
  REGION 1..32: note = FSD374
GGSGGGSLLKIWSRLIKIWTQGRRLGGSGGGS

SEQ ID NO: 285 - protein, 29 aa, synthetic construct
  REGION 1..29: note = FSD375
GGSGGGSKWKLARAFARAIKKLGGSGGGS

SEQ ID NO: 286 - protein, 26 aa, synthetic construct
  REGION 1..26: note = FSD376
GGSGGGSLARAFARAIKIFGGSGGGS

SEQ ID NO: 287 - protein, 21 aa, synthetic construct
  REGION 1..21: note = FSD377
GGGKWKLLKLWSRLLKLWGGG

SEQ ID NO: 288 - protein, 29 aa, synthetic construct
  REGION 1..29: note = FSD378
GGSGGGSKWKWIRLFSRWIRLFGGSGGGS

SEQ ID NO: 289 - protein, 30 aa, synthetic construct
  REGION 1..30: note = FSD379
KWKLSKLWSKLSKLWGGSGGGSARAARQAR

SEQ ID NO: 290 - protein, 32 aa, synthetic construct
  REGION 1..32: note = FSD381
LLKLAKALAKALKLLGGSGGGSQARAQARQAR

SEQ ID NO: 291 - protein, 26 aa, synthetic construct
  REGION 1..26: note = FSD382
LLKLAKALAKALKLLGQARAQARQAR

SEQ ID NO: 292 - protein, 29 aa, synthetic construct
  REGION 1..29: note = FSD383
LLKLLLKLLKLLGGSGGGSQARAQARQAR

SEQ ID NO: 293 - protein, 23 aa, synthetic construct
  REGION 1..23: note = FSD384
LAKALAKALKLLGQARAQARQAR

SEQ ID NO: 294 - protein, 29 aa, synthetic construct
  REGION 1..29: note = FSD385
LLKLLKLLLKLLGGSGGGSQARAQARQAR

SEQ ID NO: 295 - protein, 27 aa, synthetic construct
  REGION 1..27: note = FSD386
LLKLLKLLLKLLKLLGGGGKGGGKGGK

SEQ ID NO: 296 - protein, 18 aa, synthetic construct
  REGION 1..18: note = FSD387
QLQLLRLLLRLLKKLQLQ

SEQ ID NO: 297 - protein, 34 aa, synthetic construct
  REGION 1..34: note = FSD388
KWKLARAFSRAIKLLGGSGGGSYARALRRQARTG

SEQ ID NO: 298 - protein, 34 aa, synthetic construct
  REGION 1..34: note = FSD389
KWKLAKAFSKAIKLLGGSGGGSYAKALKKQAKTG

SEQ ID NO: 299 - protein, 17 aa, synthetic construct
  REGION 1..17: note = FSD390
KWKLWSKLLKLWSKLWK

SEQ ID NO: 300 - protein, 31 aa, synthetic construct
  REGION 1..31: note = FSD391
GGKGGKGGKWKLLKLWSRLLKLWGGKGGKGG

SEQ ID NO: 301 - protein, 31 aa, synthetic construct
  REGION 1..31: note = FSD392
GGWGGWGGKWKLLKLWSRLLKLWGGWGGWGG

SEQ ID NO: 302 - protein, 30 aa, synthetic construct
  REGION 1..30: note = FSD393
RAQRAARASGGGSGGWLKLLRSWLKLLKWK

SEQ ID NO: 303 - protein, 40 aa, synthetic construct
  REGION 1..40: note = FSD394
KWKLARAFARAIKIFGGSGGGSGGGKWKLARAFARAIKIF

SEQ ID NO: 304 - protein, 32 aa, synthetic construct
  REGION 1..32: note = FSD395
KLKLLKLLLKLLKKLGGSGGGSQAKAQAKQAK

SEQ ID NO: 305 - protein, 32 aa, synthetic construct
  REGION 1..32: note = FSD396
KLKLAKLLLKALKKLGGSGGGSQAKAQAKQAK

SEQ ID NO: 306 - protein, 32 aa, synthetic construct
  REGION 1..32: note = FSD397
KLKLAKALAKALKKLGGSGGGSQAKAQAKQAK

SEQ ID NO: 307 - protein, 32 aa, synthetic construct
  REGION 1..32: note = FSD398
KLKLLKALAKLLKKAGGSGGGSQAKAQAKQAK

SEQ ID NO: 308 - protein, 32 aa, synthetic construct
  REGION 1..32: note = FSD399
KLKLAKALLKALKKLGGSGGGSQAKAQAKQAK

SEQ ID NO: 309 - protein, 32 aa, synthetic construct
  REGION 1..32: note = FSD400
KLKAAKALAKALKALGGSGGGSQAKAQAKQAK

SEQ ID NO: 310 - protein, 37 aa, synthetic construct
  REGION 1..37: note = FSD401
GGSGGGSKWKLLKLWSRLLKLWGGSGGGSARAARQAR

SEQ ID NO: 311 - protein, 29 aa, synthetic construct
  REGION 1..29: note = FSD402
LLKLLLKLLKKLGGSGGGSQAKAQAKQAK

SEQ ID NO: 312 - protein, 29 aa, synthetic construct
  REGION 1..29: note = FSD403
LAKALAKALKKLGGSGGGSQAKAQAKQAK

SEQ ID NO: 313 - protein, 29 aa, synthetic construct
  REGION 1..29: note = FSD404
KLKLLLKLLKKLGGSGGGSQAKAQAKQAK

SEQ ID NO: 314 - protein, 30 aa, synthetic construct
  REGION 1..30: note = FSD406
KLKLLKLLLKLLKKLGGSGGGSAKAQAKQA

SEQ ID NO: 315 - protein, 30 aa, synthetic construct
  REGION 1..30: note = FSD407
KLKLLKLLLKLLKKLGGSGGGSAKAAKQAK

SEQ ID NO: 316 - protein, 25 aa, synthetic construct
  REGION 1..25: note = FSD408
KLKLLKLLLKLLKKLGGSGGGSQAG

SEQ ID NO: 317 - protein, 25 aa, synthetic construct
  REGION 1..25: note = FSD409
KLKLAKALAKALKKLGGSGGGSQAG

SEQ ID NO: 318 - protein, 32 aa, synthetic construct
  REGION 1..32: note = FSD410
KLKLLKLLLKLLKKLGGSGGGSLAKALAKLAK

SEQ ID NO: 319 - protein, 32 aa, synthetic construct
  REGION 1..32: note = FSD411
KLKLLKLLLKLLKKLGGSGGGSQAKALAKQAK

SEQ ID NO: 320 - protein, 25 aa, synthetic construct
  REGION 1..25: note = FSD412
KLKLLKLLLKLLKKLGGSGGGSLAG

SEQ ID NO: 321 - protein, 32 aa, synthetic construct
  REGION 1..32: note = FSD413
KLKLAKALAKALKKLGGSGGGSQAKALAKQAK

SEQ ID NO: 322 - protein, 36 aa, synthetic construct
  REGION 1..36: note = FSD414
LLKKLLHLLHSLLQNLKKLGGSGGGSQAKAQAKQAK

SEQ ID NO: 323 - protein, 36 aa, synthetic construct
  REGION 1..36: note = FSD415
LIRKWIHLIHSWFQNLRRLGGSGGGSQAKAQAKQAK

SEQ ID NO: 324 - protein, 29 aa, synthetic construct
  REGION 1..29: note = FSD416
GGSGGGSKWKLAKAWSRALKLWGGSGGGS

SEQ ID NO: 325 - protein, 26 aa, synthetic construct
  REGION 1..26: note = FSD417
GGSGGGSLAKAWSRALKLWGGSGGGS

SEQ ID NO: 326 - protein, 29 aa, synthetic construct
  REGION 1..29: note = FSD418
GGSGGGSKLKLLKLLLKLLKKLGGSGGGS

SEQ ID NO: 327 - protein, 29 aa, synthetic construct
  REGION 1..29: note = FSD419
GGSGGGSKLKLAKALAKALKKLGGSGGGS

SEQ ID NO: 328 - protein, 33 aa, synthetic construct
  REGION 1..33: note = FSD421
GGSGGGSLLKKLLHLLHSLLQNLKKLGGSGGGS

SEQ ID NO: 329 - protein, 27 aa, synthetic construct
  REGION 1..27: note = FSD422
HHHHHHKWKLARAFARAIKKLHHHHHH

SEQ ID NO: 330 - protein, 24 aa, synthetic construct
  REGION 1..24: note = FSD423
HHHHHHLARAFARAIKIFHHHHHH

SEQ ID NO: 331 - protein, 29 aa, synthetic construct
  REGION 1..29: note = FSD424
KWKLARAFARAIKKLGGSGGGSGGSGGGS

SEQ ID NO: 332 - protein, 32 aa, synthetic construct
  REGION 1..32: note = FSD425
KLKLAKALAKALKLLGGSGGGSQAKAQAKQAK

SEQ ID NO: 333 - protein, 33 aa, synthetic construct
  REGION 1..33: note = FSD426
KLKLAKALAKALKKLGGSGGGSKKLKAKKALKA

SEQ ID NO: 334 - protein, 30 aa, synthetic construct
  REGION 1..30: note = FSD427
LAKALAKALKKLGGSGGGSKKLKAKKALKA

SEQ ID NO: 335 - protein, 30 aa, synthetic construct
  REGION 1..30: note = FSD428
KLKLAKALAKALKKLGGSGGGSKKLKAKKA

SEQ ID NO: 336 - protein, 28 aa, synthetic construct
  REGION 1..28: note = FSD429
KLKLAKALAKALKKLGGSGGGSKKLKAK

SEQ ID NO: 337 - protein, 33 aa, synthetic construct
  REGION 1..33: note = FSD430
KLKLAKALAKALKLLGGSGGGSKKLKAKLALKA

SEQ ID NO: 338 - protein, 34 aa, synthetic construct
  REGION 1..34: note = FSD431
KWKLAKAFAKAIKKLGGSGGGSYAKALKKQAKTG

SEQ ID NO: 339 - protein, 32 aa, synthetic construct
  REGION 1..32: note = FSD432
KWKLARAFARAIKKLGGSGGGSQAKAQAKQAK

SEQ ID NO: 340 - protein, 34 aa, synthetic construct
  REGION 1..34: note = FSD433
KLKLAKALAKALKKLGGSGGGSYARALRRQARTG

SEQ ID NO: 341 - protein, 34 aa, synthetic construct
  REGION 1..34: note = FSD434
KWKLAKAFAKAIKKLGGSGGGSGGKGGKKQGKTG

SEQ ID NO: 342 - protein, 32 aa, synthetic construct
  REGION 1..32: note = FSD435
  VARIANT 1: note = X is L-2,4-diaminobutyric acid
  VARIANT 3: note = X is L-2,4-diaminobutyric acid
  VARIANT 6: note = X is L-2,4-diaminobutyric acid
  VARIANT 10: note = X is L-2,4-diaminobutyric acid
  VARIANT 13..14: note = X is L-2,4-diaminobutyric acid
  VARIANT 25: note = X is L-2,4-diaminobutyric acid
  VARIANT 29: note = X is L-2,4-diaminobutyric acid
  VARIANT 32: note = X is L-2,4-diaminobutyric acid
XLXLLXLLLXLLXXLGGSGGGSQAXAQAXQAX

SEQ ID NO: 343 - protein, 22 aa, synthetic construct
  REGION 1..22: note = FSD436
  VARIANT 5: note = X is (2-naphthyl)-L-alanine
  VARIANT 12: note = X is (2-naphthyl)-L-alanine
LARAXARAIKIXGQRRLKAKRA

SEQ ID NO: 344 - protein, 34 aa, synthetic construct; identical to SEQ ID NO: 13
  REGION 1..34: note = FSD438
  SITE 1: note = N-ter octanoic acid

SEQ ID NO: 345 - protein, 5 aa, synthetic construct
  REGION 1..5: note = Description of Artificial Sequence: Synthetic peptide
  VARIANT 2: note = X is K or R
AXAGA

SEQ ID NO: 346 - protein, 5 aa, synthetic construct
  REGION 1..5: note = Description of Artificial Sequence: Synthetic peptide
  VARIANT 2: note = X is K or R
AXAAA

SEQ ID NO: 347 - protein, 20 aa, synthetic construct
  REGION 1..20: note = Description of Artificial Sequence: Synthetic peptide
  VARIANT 1..20: note = This sequence may encompass 1-20 residues
GGGGGGGGGGGGGGGGGGGG

SEQ ID NO: 348 - protein, 20 aa, synthetic construct
  REGION 1..20: note = Description of Artificial Sequence: Synthetic peptide
  VARIANT 1..20: note = This sequence may encompass 1-20 residues
SSSSSSSSSSSSSSSSSSSS

SEQ ID NO: 349 - protein, 800 aa, synthetic construct
  VARIANT 1..800: note = This sequence may encompass 1-20 "(Gly)n(Ser)n" repeating units, where n=1-20
  REGION 1..800: note = Description of Artificial Sequence: Synthetic polypeptide
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSS

SEQ ID NO: 350 - protein, 820 aa, synthetic construct
  VARIANT 1..820: note = This sequence may encompass 1-20 "(Gly)n(Ser)n" repeating units, where n=1-20
  REGION 1..820: note = Description of Artificial Sequence: Synthetic polypeptide
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGG

SEQ ID NO: 351 - protein, 820 aa, synthetic construct
  VARIANT 1..820: note = This sequence may encompass 1-20 "(Gly)n(Ser)n" repeating units, where n=1-20
  REGION 1..820: note = Description of Artificial Sequence: Synthetic polypeptide
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSSSSSSSSSSSSSSSSSSSSS

SEQ ID NO: 352 - protein, 1620 aa, synthetic construct
  VARIANT 1..1620: note = This sequence may encompass 1-20 "(Gly)n(Ser)n" repeating units, where n=1-20
  REGION 1..1620: note = Description of Artificial Sequence: Synthetic polypeptide
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSS

SEQ ID NO: 353 - protein, 1620 aa, synthetic construct
  VARIANT 1..1620: note = This sequence may encompass 1-20 "(Gly)n(Ser)n" repeating units, where n=1-20
  REGION 1..1620: note = Description of Artificial Sequence: Synthetic polypeptide
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSS

SEQ ID NO: 354 - protein, 7 aa, synthetic construct
  REGION 1..7: note = Description of Artificial Sequence: Synthetic peptide
GGSGGGS

SEQ ID NO: 355 - protein, 11 aa, synthetic construct
  REGION 1..11: note = Description of Artificial Sequence: Synthetic peptide
GGSGGGSGGGS

SEQ ID NO: 356 - protein, 5 aa, synthetic construct
  REGION 1..5: note = Description of Artificial Sequence: Synthetic peptide
  VARIANT 1..5: note = This sequence may encompass 1-5 residues
GGGGG

SEQ ID NO: 357 - protein, 5 aa, synthetic construct
  REGION 1..5: note = Description of Artificial Sequence: Synthetic peptide
  VARIANT 1..5: note = This sequence may encompass 1-5 residues
SSSSS

SEQ ID NO: 358 - protein, 50 aa, synthetic construct
  REGION 1..50: note = Description of Artificial Sequence: Synthetic polypeptide
  VARIANT 1..50: note = This sequence may encompass 1-5 "(Gly)n(Ser)n" repeating units, where n=1-5
GGGGGSSSSSGGGGGSSSSSGGGGGSSSSSGGGGGSSSSSGGGGGSSSSS

SEQ ID NO: 359 - protein, 55 aa, synthetic construct
  REGION 1..55: note = Description of Artificial Sequence: Synthetic polypeptide
  VARIANT 1..50: note = This region may encompass 1-5 "(Gly)n(Ser)n" repeating units, where n=1-5
GGGGGSSSSSGGGGGSSSSSGGGGGSSSSSGGGGGSSSSSGGGGGSSSSSGGGGG

SEQ ID NO: 360 - protein, 55 aa, synthetic construct
  REGION 1..55: note = Description of Artificial Sequence: Synthetic polypeptide
  VARIANT 1..50: note = This region may encompass 1-5 "(Gly)n(Ser)n" repeating units, where n=1-5
GGGGGSSSSSGGGGGSSSSSGGGGGSSSSSGGGGGSSSSSGGGGGSSSSSSSSSS

SEQ ID NO: 361 - protein, 105 aa, synthetic construct
  REGION 1..105: note = Description of Artificial Sequence: Synthetic polypeptide
  VARIANT 1..105: note = This region may encompass 1-5 "(Gly)n(Ser)n" repeating units, where n=1-5
GGGGGSSSSSGGGGGSSSSSGGGGGSSSSSGGGGGSSSSSGGGGGSSSSSGGGGGGGGGGSSSSSGGGGGSSSSSGGGGGSSSSSGGGGGSSSSSGGGGGSSSSS

SEQ ID NO: 362 - protein, 105 aa, synthetic construct
  REGION 1..105: note = Description of Artificial Sequence: Synthetic polypeptide
  VARIANT 1..105: note = This region may encompass 1-5 "(Gly)n(Ser)n" repeating units, where n=1-5
GGGGGSSSSSGGGGGSSSSSGGGGGSSSSSGGGGGSSSSSGGGGGSSSSSSSSSSGGGGGSSSSSGGGGGSSSSSGGGGGSSSSSGGGGGSSSSSGGGGGSSSSS

SEQ ID NO: 363 - protein, 18 aa, synthetic construct
  REGION 1..18: note = Description of Artificial Sequence: Synthetic peptide
KWKLFKKIGAVLKVLTTG

SEQ ID NO: 364 - protein, 11 aa, synthetic construct
  REGION 1..11: note = Description of Artificial Sequence: Synthetic peptide
YGRKKRRQRRR

SEQ ID NO: 365 - protein, 18 aa, synthetic construct
  REGION 1..18: note = Description of Artificial Sequence: Synthetic peptide
KWKLFKKYGRKKRRQRRR

SEQ ID NO: 366 - protein, 14 aa, synthetic construct
  REGION 1..14: note = Description of Artificial Sequence: Synthetic peptide
CLLKKLLKKLLKKC

SEQ ID NO: 367 - protein, 14 aa, synthetic construct
  REGION 1..14: note = Description of Artificial Sequence: Synthetic peptide
GLLKKLLKKLLKKG

SEQ ID NO: 368 - protein, 9 aa, synthetic construct
  REGION 1..9: note = Description of Artificial Sequence: Synthetic peptide
  VARIANT 1..9: note = This sequence may encompass 2-9 residues
HHHHHHHHH

(19) United States
(12) Patent Application Publication
(10) Pub. No.: US 2026/0242417 A1
(43) Pub. Date: Aug. 20, 2026

(54) PEPTIDE-BASED NON-PROTEINACEOUS CARGO DELIVERY

(71) Applicant: Feldan Bio Inc., Quebec (CA)

(72) Inventors: David GUAY, Quebec (CA); Thomas DEL'GUIDICE, Quebec (CA); Jean-Pascal LEPETIT-STOFFAES, Quebec (CA); Xavier BARBEAU, Levis (CA); Nancy MESSIER, Quebec (CA); Stéphanie HALLÉE, Ste-Catherine-de-la-Jacques-Cartier (CA)

(21) Appl. No.: 19/312,632

(22) Filed: Aug. 28, 2025

Related U.S. Application Data
(63) Continuation of application No. 17/604,328, filed on Apr. 17, 2020, now Pat. No. 12,428,447.

(30) Foreign Application Priority Data
Apr. 18, 2019 (CA) 3040645

(51) Int. Cl. C07K 7/08; A61K 38/00; A61P 35/00; C07K 14/00; C12N 15/87; G01N 33/50

(52) U.S. Cl. CPC C07K 7/08; A61P 35/00; C07K 14/001; C12N 15/87; G01N 33/5035; A61K 38/00; C07K 2319/03

(57) ABSTRACT

Described herein are methods, compositions, kits and synthetic peptide shuttle agents relating to the transduction of proteinaceous and/or non-proteinaceous cargoes. The method generally comprises contacting target eukaryotic cells with a non-proteinaceous cargo and a concentration of a synthetic peptide shuttle agent sufficient to increase the transduction efficiency of the non-proteinaceous cargo, as compared to in the absence of said synthetic peptide shuttle agent. In embodiments, the non-proteinaceous cargo may be a drug, such as a small molecule drug, for treating a disease. In other embodiments, novel synthetic peptide shuttle agents having transduction activity for proteinaceous and/or non-proteinaceous cargoes are described, as well as the use of propidium iodide or other membrane-impermeable fluorescent DNA intercalating agent as a surrogate cargo for selecting versatile synthetic peptide shuttle agents having transduction activity for both proteinaceous and non-proteinaceous cargoes.

The present description relates to the intracellular delivery of non-proteinaceous cargoes. More specifically, the present description relates to the use of synthetic peptide shuttle agents for the intracellular delivery of small molecules and other non-proteinaceous cargoes, as well as improved synthetic peptide shuttle agents having transduction activity for both proteins and small molecules.

The present description refers to a number of documents, the contents of which are herein incorporated by reference in their entirety.

### CROSS REFERENCE

This application is a continuation of U.S. application Ser. No. 17/604,328, filed Oct. 15, 2021, which is a National Phase application of International Application No PCT/CA2020/050517, filed on Apr. 17, 2020, which claims the benefit of Canadian Application No. 3,040,645, filed on Apr. 18, 2019, each of which is incorporated herein by reference in its entirety.

### SEQUENCE LISTING

This application contains a Sequence Listing which has been submitted electronically in XML format and is hereby incorporated by reference in its entirety. The XML copy, created on Mar. 4, 2026, is named AMENDED_SEQUENCE_LISTING_49446-707301.xml and is 483,676 bytes in size.

### BACKGROUND

Most drugs have traditionally been small molecule organic compounds that are sufficiently small and lipophilic to pass through cellular membranes to engage intracellular targets. During conventional drug discovery processes, small molecule drug candidates are routinely selected based not only on their affinity for their biological targets, but also on their drug-like physicochemical properties that, amongst other things, govern their ability to be delivered intracellularly and reach their biological targets. Thus, under conventional drug development ideologies, compounds identified in large-scale screening efforts as showing high target binding affinity and specificity may be ultimately discarded as clinical drug candidates because of their diminished ability to be delivered intracellularly. Furthermore, even cell membrane-permeable compounds may benefit from improved intracellular/cytosolic delivery, for example to increase speed of uptake and/or reduce the concentration administered to obtain the desired biological effect. There is therefore a need for technologies that can facilitate the intracellular/cytosolic delivery of small molecule cargoes to provide greater flexibility in terms of drug design and perhaps open the door for the use of novel therapeutic compounds that may otherwise have been disregarded based on traditional small molecule drug design.

### SUMMARY

Synthetic peptide shuttle agents represent a recently defined family of peptides previously reported to quickly and efficiently transduce proteinaceous cargoes to the cytosol and/or nucleus of a wide variety of target eukaryotic cells. The first generation of such peptide shuttle agents were described in WO/2016/161516, wherein the peptide shuttle agents comprise an endosome leakage domain (ELD) operably linked to a cell penetrating domain (CPD). WO/2018/068135 subsequently described further synthetic peptide shuttle agents rationally-designed based on a set of fifteen design parameters for the purpose of improving the transduction of proteinaceous cargoes, while reducing toxicity of the first generation peptide shuttle agents. The present disclosure relates to the discovery that such synthetic peptide shuttle agents, previously reported to transduce large proteinaceous cargoes, also generally have the ability of quickly and efficiently transducing smaller, non-proteinaceous cargoes (e.g., small molecule organic compounds). The experimental results presented in Example 2 show that synthetic peptide shuttle agents, including representative members of the shuttle agents described in WO/2016/161516 and WO/2018/068135, as well as additional rationally-designed shuttle agents, are able to transduce the membrane impermeable fluorescent dye propidium iodide (PI), which can be considered as a small molecule organic compound cargo. Strikingly, negative control peptides that fail to respect key rational-design parameters described in WO/2018/068135 for the delivery of proteinaceous cargoes also failed to transduce PI, suggesting that the rational-design parameters of WO/2018/068135 for proteinaceous cargo delivery may also generally apply to the design of peptide shuttle agents for the delivery of non-proteinaceous cargoes. In Example 3, it is shown that a representative synthetic peptide shuttle agent not only enables intracellular delivery of structurally unrelated small molecule inhibitors of the HedgeHog signalling pathway into cultured cells, but that the delivered inhibitors are free to bind to their intracellular targets and exert their inhibitory activity. In Example 4, it is shown that a representative synthetic peptide shuttle agent enables in vivo delivery and activity of small molecule inhibitors of HedgeHog signalling following topical application in shaved mice. In Example 5, it is shown that a different representative synthetic peptide shuttle agent enables intracellular delivery of a membrane-impermeable small molecule compound that is a sodium channel inhibitor (QX-314), resulting in an associated reduction in evoked current amplitudes as measured by patch-clamping. Finally, Examples 6 and 7 show the results of a large-scale screening of over 300 candidate peptide shuttle agents for PI and GFP-NLS transduction activity, and reveal a striking correlation between PI transduction efficiency and GFP-NLS transduction efficiency, suggesting that robust PI transduction predicts shuttle agents having proteinaceous cargo transduction activity.

In some aspects, described herein is a method for non-proteinaceous cargo transduction, the method comprising contacting target eukaryotic cells with a non-proteinaceous cargo and a concentration of a synthetic peptide shuttle agent sufficient to increase the transduction efficiency of said non-proteinaceous cargo, as compared to in the absence of said synthetic peptide shuttle agent.

In some aspects, described herein is a composition for use in transducing a non-proteinaceous cargo into target eukaryotic cells, the composition comprising a synthetic peptide shuttle agent formulated with a pharmaceutically suitable excipient, wherein the concentration of the synthetic peptide shuttle agent in the composition is sufficient to increase the transduction efficiency and cytosolic and/or nuclear delivery of the non-proteinaceous cargo into said target eukaryotic cells upon administration, as compared to in the absence of said synthetic peptide shuttle agent.

In some aspects, describe herein is a composition for use in therapy, the composition comprising a synthetic peptide shuttle agent formulated with a non-proteinaceous cargo (e.g., therapeutically or biologically active non-proteinaceous cargo) to be transduced into target eukaryotic cells by the synthetic peptide shuttle agent, wherein the concentration of the synthetic peptide shuttle agent in the composition is sufficient to increase the transduction efficiency and cytosolic and/or nuclear delivery of the non-proteinaceous cargo into said target eukaryotic cells upon administration, as compared to in the absence of said synthetic peptide shuttle agent.

In some aspects, described herein is a synthetic peptide shuttle agent having transduction activity for both proteinaceous and non-proteinaceous cargoes, the shuttle agent comprising or consisting of the amino acid sequence any one of SEQ ID NOs: 1 to 50. In some aspects, described herein is a synthetic peptide shuttle agent having transduction activity for both proteinaceous and non-proteinaceous cargoes, the shuttle agent comprising or consisting of an amino acid sequence that differs from any one of SEQ ID NOs: 1 to 50 by no more than 1, 2, 3, 4, 5, 6, 7, 8, 9, or 10 amino acids (e.g., excluding any linker domains, such as flexible serine/glycine-rich linker domains). In some aspects, described herein is a synthetic peptide shuttle agent having transduction activity for both proteinaceous and non-proteinaceous cargoes, the shuttle agent comprising or consisting of an amino acid sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 1 to 50 (e.g., calculated excluding any linker domains, such as flexible serine/glycine-rich linker domains).

In some aspects, described herein is a synthetic peptide shuttle agent having transduction activity for both proteinaceous and non-proteinaceous cargoes in target eukaryotic cells, the shuttle agent being:
(1) a peptide at least 17, 18, 19, or 20 amino acids in length comprising
(2) an amphipathic alpha-helical motif having
(3) a positively-charged hydrophilic outer face, and a hydrophobic outer face,
   wherein at least five of the following parameters (4) to (15) are respected:
(4) the hydrophobic outer face comprises a highly hydrophobic core consisting of spatially adjacent L, I, F, V, W, and/or M amino acids representing 12 to 50% of the amino acids of the peptide, based on an open cylindrical representation of the alpha-helix having 3.6 residues per turn;
(5) the peptide has a hydrophobic moment (μ) of 3.5 to 11;
(6) the peptide has a predicted net charge of at least +4 at physiological pH;
(7) the peptide has an isoelectric point (pI) of 8 to 13;
(8) the peptide is composed of 35% to 65% of any combination of the amino acids: A, C, G, I, L, M, F, P, W, Y, and V;
(9) the peptide is composed of 0% to 30% of any combination of the amino acids: N, Q, S, and T;
(10) the peptide is composed of 35% to 85% of any combination of the amino acids: A, L, K, or R;
(11) the peptide is composed of 15% to 45% of any combination of the amino acids: A and L, provided there being at least 5% of L in the peptide;
(12) the peptide is composed of 20% to 45% of any combination of the amino acids: K and R;
(13) the peptide is composed of 0% to 10% of any combination of the amino acids: D and E;
(14) the difference between the percentage of A and L residues in the peptide (% A+L), and the percentage of K and R residues in the peptide (K+R), is less than or equal to 10%; and
(15) the peptide is composed of 10% to 45% of any combination of the amino acids: Q, Y, W, P, I, S, G, V, F, E, D, C, M, N, T and H,
   wherein the shuttle agent increases the transduction efficiency of propidium iodide or other membrane-impermeable fluorescent DNA intercalating agent by at least 3, 3.5, 4, 4.5, 5, 5.5, 6, 6.5, 7, 7.5, 8, 8.5, 9, 9.5, or 10-fold over a corresponding negative control lacking said shuttle agent, and/or enables a transduction efficiency of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of propidium iodide or other membrane-impermeable fluorescent DNA intercalating agent, in a eukaryotic cell line model (e.g., HeLa) suitable for assessing cargo transduction in said target eukaryotic cells.

In some aspects, described herein is a synthetic peptide shuttle agent having transduction activity for both proteinaceous and non-proteinaceous cargoes in target eukaryotic cells, wherein the shuttle agent comprises or consists of: (a) the amino acid sequence any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, or 344; or (b) an amino acid sequence that differs from (a) by only conservative amino acid substitutions (e.g., by no more than no more than 1, 2, 3, 4, 5, 6, 7, 8, 9, or 10 conservative amino acid substitutions, preferably excluding any linker domains, such as flexible serine/glycine-rich linker domains), wherein the shuttle agent: increases the transduction efficiency of propidium iodide or other membrane-impermeable fluorescent DNA intercalating agent by at least 3, 3.5, 4, 4.5, 5, 5.5, 6, 6.5, 7, 7.5, 8, 8.5, 9, 9.5, or 10-fold over a corresponding negative control lacking said shuttle agent; and/or enables a transduction efficiency of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of propidium iodide or other membrane-impermeable fluorescent DNA intercalating agent, in a eukaryotic cell line model (e.g., HeLa) suitable for assessing cargo transduction in said target eukaryotic cells.

In some aspects, described herein is a synthetic peptide shuttle agent having proteinaceous cargo transduction activity in target eukaryotic cells, wherein the shuttle agent comprises or consists of: (a) the amino acid sequence any one of SEQ ID NOs: 52, 57, 79, 108, 140, 147, 148, 173, 241, 261, 286, 295, 301, 309, 312, 325, 333-337, or 343; or (b) an amino acid sequence that differs from (a) by only conservative amino acid substitutions (e.g., by no more than no more than 1, 2, 3, 4, 5, 6, 7, 8, 9, or 10 conservative amino acid substitutions, preferably excluding any linker domains, such as flexible serine/glycine-rich linker domains), wherein the shuttle agent: increases the transduction efficiency of GFP-NLS by at least 3, 3.5, 4, 4.5, 5, 5.5, 6, 6.5, 7, 7.5, 8, 8.5, 9, 9.5, or 10-fold over a corresponding negative control lacking said shuttle agent, and/or enables a transduction efficiency of at least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% (e.g., as determined by flow cytometry) of GFP-NLS in a eukaryotic cell line model (e.g., HeLa) suitable for assessing cargo transduction in said target eukaryotic cells.

In some aspects, described herein is a synthetic peptide shuttle agent variant having transduction activity for proteinaceous and/or non-proteinaceous cargoes in target eukaryotic cells, the synthetic peptide shuttle agent variant being identical to any one of the synthetic peptide shuttle agents as defined herein, except having at least one amino acid being replaced with a corresponding synthetic amino acid having a side chain of similar physiochemical properties (e.g., structure, hydrophobicity, or charge) as the amino acid being replaced, wherein the shuttle agent variant increases the transduction efficiency of said cargo in target eukaryotic cells, as compared to in the absence of the shuttle agent variant.

In some aspects, described herein is an in vitro or in vivo method for proteinaceous and/or non-proteinaceous cargo transduction, the method comprising contacting target eukaryotic cells with the cargo and a concentration of the synthetic peptide shuttle agent or synthetic peptide shuttle agent variant as defined herein sufficient to increase the transduction efficiency of the cargo into the target eukaryotic cells, as compared to in the absence of said synthetic peptide shuttle agent.

In some aspects, described herein is a composition for use in therapy, the composition comprising the synthetic peptide shuttle agent or synthetic peptide shuttle agent variant as defined herein formulated with a proteinaceous and/or non-proteinaceous cargo to be transduced into target eukaryotic cells by the synthetic peptide shuttle agent, wherein the concentration of the synthetic peptide shuttle agent or synthetic peptide shuttle agent variant in the composition is sufficient to increase the transduction efficiency and cytosolic delivery of the cargo into said target eukaryotic cells upon administration, as compared to in the absence of said synthetic peptide shuttle agent.

In some aspects, described herein is a kit comprising the synthetic peptide shuttle agent or synthetic peptide shuttle agent variant as defined herein, and a proteinaceous and/or non-proteinaceous cargo to be transduced by the synthetic peptide shuttle agent or synthetic peptide shuttle agent variant.

In some aspects, described herein a process for producing a candidate synthetic peptide shuttle agent expected to have transduction activity for a cargo of interest in target eukaryotic cells, the method comprising synthesizing a peptide which is: (1) a peptide at least 17, 18, 19, or 20 amino acids in length comprising (2) an amphipathic alpha-helical motif having (3) a positively-charged hydrophilic outer face, and a hydrophobic outer face, wherein at least five of the parameters (4) to (15) defined herein are respected, wherein the shuttle agent increases the transduction efficiency of propidium iodide or other membrane-impermeable fluorescent DNA intercalating agent by at least 3, 3.5, 4, 4.5, 5, 5.5, 6, 6.5, 7, 7.5, 8, 8.5, 9, 9.5, or 10-fold over a corresponding negative control lacking said shuttle agent, and/or enables a transduction efficiency of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of propidium iodide or other membrane-impermeable fluorescent DNA intercalating agent, in a eukaryotic cell line model (e.g., HeLa) suitable for assessing cargo transduction in said target eukaryotic cells.

In some aspects, described herein an in vitro or in vivo method for identifying, qualifying, or selecting a synthetic peptide shuttle agent expected to have transduction activity for both proteinaceous and non-proteinaceous cargoes in target eukaryotic cells, the method comprising: providing model eukaryotic cells or a model organism suitable for assessing cargo transduction in the target eukaryotic cells; providing a candidate synthetic peptide shuttle agent (e.g., as defined herein); and measuring the transduction activity (e.g., transduction efficiency, such as by flow cytometry) of the candidate synthetic peptide shuttle agent to transduce propidium iodide or other membrane-impermeable fluorescent DNA intercalating agent into the model eukaryotic cells or model organism, wherein the candidate shuttle agent is expected to have transduction activity for both proteinaceous and non-proteinaceous cargoes in the target eukaryotic cells when the transduction activity (e.g., transduction efficiency) of propidium iodide or other membrane-impermeable fluorescent DNA intercalating agent is increased by at least 3, 3.5, 4, 4.5, 5, 5.5, 6, 6.5, 7, 7.5, 8, 8.5, 9, 9.5, or 10-fold over a corresponding negative control lacking the candidate synthetic peptide shuttle agent, and/or a transduction efficiency of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of the propidium iodide or other membrane-impermeable fluorescent DNA intercalating agent occurs, in the model eukaryotic cells or model organism.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, “protein” or “polypeptide” or “peptide” means any peptide-linked chain of amino acids, which may or may not comprise any type of modification (e.g., chemical or post-translational modifications such as acetylation, phosphorylation, glycosylation, sulfatation, sumoylation, prenylation, ubiquitination, etc.). For further clarity, protein/polypeptide/peptide modifications are envisaged so long as the modification does not destroy the cargo transduction activity of the shuttle agents described herein. For example, shuttle agents described herein may be linear or circular, may be synthesized with one or more D- or L-amino acids, and/or may be conjugated to a fatty acid (e.g., at their N terminus). Shuttle agents described herein may also have at least one amino acid being replaced with a corresponding synthetic amino acid having a side chain of similar physiochemical properties (e.g., structure, hydrophobicity, or charge) as the amino acid being replaced.

As used herein, a “domain” or “protein domain” generally refers to a part of a protein having a particular functionality or function. Some domains conserve their function when separated from the rest of the protein, and thus can be used in a modular fashion. The modular characteristic of many protein domains can provide flexibility in terms of their placement within the shuttle agents of the present description. However, some domains may perform better when engineered at certain positions of the shuttle agent (e.g., at the N- or C-terminal region, or therebetween). The position of the domain within its endogenous protein is sometimes an indicator of where the domain should be engineered within the shuttle agent and of what type/length of linker should be used. Standard recombinant DNA techniques can be used by the skilled person to manipulate the placement and/or number of the domains within the shuttle agents of the present description in view of the present disclosure. Furthermore, assays disclosed herein, as well as others known in the art, can be used to assess the functionality of each of the domains within the context of the shuttle agents (e.g., their ability to facilitate cell penetration across the plasma membrane, endosome escape, and/or access to the cytosol). Standard methods can also be used to assess whether the domains of the shuttle agent affect the activity of the cargo to be delivered intracellularly. In this regard, the expression “operably linked” as used herein refers to the ability of the domains to carry out their intended function(s) (e.g., cell penetration, endosome escape, and/or subcellular targeting) within the context of the shuttle agents of the present description. For greater clarity, the expression “operably linked” is meant to define a functional connection between two or more domains without being limited to a particular order or distance between same.

As used herein, the term “synthetic” used in expressions such as “synthetic peptide”, synthetic peptide shuttle agent”, or “synthetic polypeptide” is intended to refer to non-naturally occurring molecules that can be produced in vitro (e.g., synthesized chemically and/or produced using recombinant DNA technology). The purities of various synthetic preparations may be assessed by, for example, high-performance liquid chromatography analysis and mass spectroscopy. Chemical synthesis approaches may be advantageous over cellular expression systems (e.g., yeast or bacteria protein expression systems), as they may preclude the need for extensive recombinant protein purification steps (e.g., required for clinical use). In contrast, longer synthetic polypeptides may be more complicated and/or costly to produce via chemical synthesis approaches and such polypeptides may be more advantageously produced using cellular expression systems. In some embodiments, the peptides or shuttle agents of the present description may be chemically synthesized (e.g., solid- or liquid phase peptide synthesis), as opposed to expressed from a recombinant host cell. In some embodiments, the peptides or shuttle agent of the present description may lack an N-terminal methionine residue. A person of skill in the art may adapt a synthetic peptide or shuttle agent of the present description by using one or more modified amino acids (e.g., non-naturally-occurring amino acids), or by chemically modifying the synthetic peptide or shuttle agent of the present description, to suit particular needs of stability or other needs.

As used herein, the term “independent” is generally intended refer to molecules or agents which are not covalently bound to one another. For example, the expression “independent cargo” is intended to refer to a cargo to be delivered intracellularly (transduced) that is not covalently bound (e.g., not fused) to a shuttle agent of the present description. In some aspects, having shuttle agents that are independent of (not fused to) a cargo may be advantageous by providing increased shuttle agent versatility—e.g., being able to readily vary the ratio of shuttle agent to cargo (as opposed to being limited to a fixed ratio in the case of a covalent linkage between the shuttle agent and cargo).

As used herein, the expression “is or is from” or “is from” comprises functional variants of a given protein domain (e.g., CPD or ELD), such as conservative amino acid substitutions, deletions, modifications, as well as variants or function derivatives, which do not abrogate the activity of the protein domain.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1A-1D show delivery and viability results of HeLa cells co-incubated for 1 minute with different categories of synthetic peptide shuttle agents combined with a non-proteinaceous cargo (propidium iodide, PI; FIGS. 1A and 1B) or a proteinaceous cargo (GFP-NLS protein; FIGS. 1C and 1D). Results were acquired by flow cytometry two hours after cargo delivery and expressed as percentages of fluorescent cells (% PI+ cells or % GFP+ cells). Categories of peptides shown (from left to right): Synthetic peptide shuttle agents comprising an endosome leakage domain (ELD) operably linked to a cell penetrating domain (CPD) described in WO/2016/161516; Rationally-designed synthetic peptide shuttle agents described in WO/2018/068135; additional rationally-designed synthetic peptide shuttle agents described herein; Cyclic peptides described herein; and Negative control peptides that fail to respect several rational-design parameters set forth in WO/2018/068135. In FIG. 1A, “FS then PI” indicates that PI was added 1 hour after the treatment with the synthetic peptide shuttle agents, ensuring that PI-positive signal is not due to cell death. “Negative control” are cells incubated with cargo alone (“PI” in FIGS. 1A and 1B or “GFP-NLS” in FIGS. 1C and 1D), or untreated cells that were not exposed to the cargo or peptide shuttle agents (“NT”, FIG. 1A-1D).

FIG. 2 is a table summarizing the results in FIG. 1A-1D.

FIG. 3 shows the activity of small molecule inhibitors of HedgeHog signalling (Gant61, HPI-4, Itraconazole, or ATO) transduced into NIH3T3 Gli-luciferase reporter cells by the peptide shuttle agent FSD250D. Successful small molecule transduction in the presence of the peptide shuttle agent (“+FSD250D”; SEQ ID NO: 36) resulted in reduced luminescence intensity of the NIH3T3 Gli-luciferase reporter cells stimulated with recombinant mouse Sonic HedgeHog protein (+mShh), as compared to in the absence of the peptide shuttle agent (“−FSD250D”).

FIG. 4 shows the successful in vivo transduction of small molecule inhibitors of HedgeHog signalling (Gant61 and Itraconazole) in skin cells of shaved mice by the peptide shuttle agent FSD250D. Depilation of mouse skin induces hair growth associated with a strong induction of the HedgeHog pathway. This experiment consisted of activating the HedgeHog pathway in mice by depilation, and then measuring the delay in hair regrowth by delivering in the skin cells small molecule HedgeHog pathway inhibitors (Gant61 or Itraconazole) that bind to intracellular targets. The results show that mice treated with the small molecule HedgeHog inhibitors Gant61 or Itraconazole in the presence of FSD250D (“FSD250D+Gant61 100 μM” and “FSD250D+Itraconazole 100 μM”) showed delayed hair regrowth at 10 days post-treatment (*), as compared to in the absence of FSD250D (“Gant61 100 μM” and “Itraconazole 100 μM”), or in the presence of the shuttle peptide alone (“FSD250D”).

FIG. 5A-5C shows representative patch-clamp electrophysiology whole-cell current traces of HEK293 cells stably expressing the sodium channel Nav1.7 upon exposure to the membrane impermeable sodium channel inhibitor QX-314 with or without FSD194. Reduction of the current amplitude was observed when cells were transiently exposed to QX-314 and GFP-NLS in the presence of FSD194 (i.e., 1 mM QX-314+15 μM GFP-NLS+5 μM FSD194), consistent with the presence of QX-314 inside the cells (FIG. 5C). This same current amplitude reduction was not observed in the absence of QX-314 (i.e., 15 μM GFP-NLS+5 μM FSD194+; FIG. 2A) or in the absence of FSD194 (i.e., 2.5 mM QX-314+15 μM GFP-NLS; FIG. 2B). Furthermore, GFP-NLS-positive cells were identified in the QX-314+GFP-NLS+FSD194 and in the FSD194+GFP-NLS conditions, but not in the QX-314+GFP-NLS conditions, indicating that GFP-NLS was indeed co-transduced along with the QX-314 by the peptide shuttle agent.

FIG. 6 and FIG. 7 show the results of a large-scale screening of over 300 candidate peptide shuttle agents for PI and GFP-NLS transduction activity. FIG. 6 shows results of all candidate peptide shuttle agents screened that had a mean PI transduction efficiency of 10% or higher, sorted based on their level of mean PI transduction efficiency. FIG. 7 shows results of all candidate peptide shuttle agents screened that had a mean PI transduction efficiency of under 10% and a mean GFP-NLS transduction efficiency of at least 7%, sorted based on their level of mean GFP-NLS transduction efficiency.

| SEQ ID | |
|---|---|
| NO: | Description |
| 1 | CM18-Penetratin- |
| | cys |
| 2 | TAT-KALA |
| 3 | His-CM18-PTD4 |
| 4 | His-LAH4-PTD4 |
| 5 | PTD4-KALA |
| 6 | EB1-PTD4 |
| 7 | His-CM18-PTD4- |
| | 6Cys |
| 8 | CM18-PTD4 |
| 9 | CM18-PTD4- |
| | 6His |
| 10 | His-CM18-PTD4- |
| | His |
| 11 | TAT-CM18 |
| 12 | FSD5 |
| 13 | FSD10 |
| 14 | FSD12 |
| 15 | FSD18 |
| 16 | FSD19 |
| 17 | FSD21 |
| 18 | FSD23 |
| 19 | FSD120* |
| 20 | FSD127* |
| 21 | FSD129* |
| 22 | FSD131* |
| 23 | FSD134* |
| 24 | FSD146* |
| 25 | FSD155* |
| 26 | FSD156* |
| 27 | FSD157* |
| 28 | FSD159* |
| 29 | FSD162* |
| 30 | FSD168* |
| 31 | FSD173* |
| 32 | FSD174* |
| 33 | FSD194* |
| 34 | FSD220* |
| 35 | FSD250* |
| 36 | FSD250D* |
| 37 | FSD253* |
| 38 | FSD258* |
| 39 | FSD262* |
| 40 | FSD263* |
| 41 | FSD264* |
| 42 | FSD265* |
| 43 | FSD268* |
| 44 | FSD286* |
| 45 | FSD271* |
| 46 | FSD272* |
| 47 | FSD273* |
| 48 | FSD276* |
| 49 | FSD268 Cyclic |
| | Amide* |
| 50 | FSD268 Cyclic |
| | Disulfide* |
| 51 | FSD10 Scramble |
| 52 | FSD268 |
| | Scramble* |
| 53 | FSD174 |
| | Scramble* |
| 54 | FSN3 |
| 55 | FSN4 |
| 56 | FSN7 |
| 57 | FSN8 |
| 58 | FSD117 |
| 59 | FSD118 |
| 60 | FSD119 |
| 61 | FSD121 |
| 62 | FSD122 |
| 63 | FSD123 |
| 64 | FSD124 |
| 65 | FSD125 |
| 66 | FSD126 |
| 67 | FSD127 |
| 68 | FSD128 |
| 69 | FSD130 |
| 70 | FSD132 |
| 71 | FSD133 |
| 72 | FSD135 |
| 73 | FSD137 |
| 74 | FSD138 |
| 75 | FSD139 |
| 76 | FSD140 |
| 77 | FSD141 |
| 78 | FSD142 |
| 79 | FSD143 |
| 80 | FSD144 |
| 81 | FSD145 |
| 82 | FSD147 |
| 83 | FSD148 |
| 84 | FSD149 |
| 85 | FSD150 |
| 86 | FSD151 |
| 87 | FSD152 |
| 88 | FSD153 |
| 89 | FSD154 |
| 90 | FSD158 |
| 91 | FSD160 |
| 92 | FSD161 |
| 93 | FSD163 |
| 94 | FSD164 |
| 95 | FSD165 |
| 96 | FSD166 |
| 97 | FSD167 |
| 98 | FSD169 |
| 99 | FSD170 |
| 100 | FSD171 |
| 101 | FSD172 |
| 102 | FSD175 |
| 103 | FSD176 |
| 104 | FSD177 |
| 105 | FSD178 |
| 106 | FSD179 |
| 107 | FSD180 |
| 108 | FSD181 |
| 109 | FSD182 |
| 110 | FSD183 |
| 111 | FSD184 |
| 112 | FSD185 |
| 113 | FSD186 |
| 114 | FSD187 |
| 115 | FSD188 |
| 116 | FSD189 |
| 117 | FSD190 |
| 118 | FSD191 |
| 119 | FSD192 |
| 120 | FSD193 |
| 121 | FSD195 |
| 122 | FSD196 |
| 123 | FSD197 |
| 124 | FSD198 |
| 125 | FSD199 |
| 126 | FSD200 |
| 127 | FSD201 |
| 128 | FSD202 |
| 129 | FSD203 |
| 130 | FSD204 |
| 131 | FSD205 |
| 132 | FSD206 |
| 133 | FSD207 |
| 134 | FSD208 |
| 135 | FSD209 |
| 136 | FSD210 |
| 137 | FSD211 |
| 138 | FSD212 |
| 139 | FSD213 |
| 140 | FSD214 |
| 141 | FSD215 |
| 142 | FSD216 |
| 143 | FSD217 |
| 144 | FSD218 |
| 145 | FSD219 |
| 146 | FSD221 |
| 147 | FSD222 |
| 148 | FSD223 |
| 149 | FSD224 |
| 150 | FSD225 |
| 151 | FSD226 |
| 152 | FSD227 |
| 153 | FSD228 |
| 154 | FSD229 |
| 155 | FSD230 |
| 156 | FSD231 |
| 157 | FSD232 |
| 158 | FSD233 |
| 159 | FSD234 |
| 160 | FSD235 |
| 161 | FSD236 |
| 162 | FSD237 |
| 163 | FSD238 |
| 164 | FSD239 |
| 165 | FSD240 |
| 166 | FSD241 |
| 167 | FSD243 |
| 168 | FSD244 |
| 169 | FSD246 |
| 170 | FSD247 |
| 171 | FSD248 |
| 172 | FSD250 Scramble |
| 173 | FSD250E |
| 174 | FSD251 |
| 175 | FSD254 |
| 176 | FSD255 |
| 177 | FSD256 |
| 178 | FSD257 |
| 179 | FSD259 |
| 180 | FSD260 |
| 181 | FSD261 |
| 182 | FSD266 |
| 183 | FSD267 |
| 184 | FSD269 |
| 185 | FSD270 |
| 186 | FSD274 |
| 187 | FSD275 |
| 188 | FSD276 |
| 189 | FSD277 |
| 190 | FSD278 |
| 191 | FSD279 |
| 192 | FSD280 |
| 193 | FSD281 |
| 194 | FSD282 |
| 195 | FSD283 |
| 196 | FSD284 |
| 197 | FSD285 |
| 198 | FSD287 |
| 199 | FSD288 |
| 200 | FSD289 |
| 201 | FSD290 |
| 202 | FSD291 |
| 203 | FSD292 |
| 204 | FSD293 |
| 205 | FSD294 |
| 206 | FSD295 |
| 207 | FSD296 |
| 208 | FSD297 |
| 209 | FSD298 |
| 210 | FSD299 |
| 211 | FSD300 |
| 212 | FSD301 |
| 213 | FSD302 |
| 214 | FSD303 |
| 215 | FSD304 |
| 216 | FSD305 |
| 217 | FSD306 |
| 218 | FSD307 |
| 219 | FSD308 |
| 220 | FSD309 |
| 221 | FSD310 |
| 222 | FSD311 |
| 223 | FSD312 |
| 224 | FSD313 |
| 225 | FSD314 |
| 226 | FSD315 |
| 227 | FSD316 |
| 228 | FSD317 |
| 229 | FSD318 |
| 230 | FSD319 |
| 231 | FSD320 |
| 232 | FSD321 |
| 233 | FSD322 |
| 234 | FSD323 |
| 235 | FSD324 |
| 236 | FSD325 |
| 237 | FSD326 |
| 238 | FSD327 |
| 239 | FSD328 |
| 240 | FSD330 |
| 241 | FSD331 |
| 242 | FSD332 |
| 243 | FSD333 |
| 244 | FSD334 |
| 245 | FSD335 |
| 246 | FSD336 |
| 247 | FSD337 |
| 248 | FSD338 |
| 249 | FSD339 |
| 250 | FSD340 |
| 251 | FSD341 |
| 252 | FSD342 |
| 253 | FSD343 |
| 254 | FSD344 |
| 255 | FSD345 |
| 256 | FSD346 |
| 257 | FSD347 |
| 258 | FSD348 |
| 259 | FSD349 |
| 260 | FSD350 |
| 261 | FSD351 |
| 262 | FSD352 |
| 263 | FSD353 |
| 264 | FSD354 |
| 265 | FSD355 |
| 266 | FSD356 |
| 267 | FSD357 |
| 268 | FSD358 |
| 269 | FSD359 |
| 270 | FSD360 |
| 271 | FSD361 |
| 272 | FSD362 |
| 273 | FSD363 |
| 274 | FSD364 |
| 275 | FSD365 |
| 276 | FSD366 |
| 277 | FSD367 |
| 278 | FSD368 |
| 279 | FSD369 |
| 280 | FSD370 |
| 281 | FSD371 |
| 282 | FSD372 |
| 283 | FSD373 |
| 284 | FSD374 |
| 285 | FSD375 |
| 286 | FSD376 |
| 287 | FSD377 |
| 288 | FSD378 |
| 289 | FSD379 |
| 290 | FSD381 |
| 291 | FSD382 |
| 292 | FSD383 |
| 293 | FSD384 |
| 294 | FSD385 |
| 295 | FSD386 |
| 296 | FSD387 |
| 297 | FSD388 |
| 298 | FSD389 |
| 299 | FSD390 |
| 300 | FSD391 |
| 301 | FSD392 |
| 302 | FSD393 |
| 303 | FSD394 |
| 304 | FSD395 |
| 305 | FSD396 |
| 306 | FSD397 |
| 307 | FSD398 |
| 308 | FSD399 |
| 309 | FSD400 |
| 310 | FSD401 |
| 311 | FSD402 |
| 312 | FSD403 |
| 313 | FSD404 |
| 314 | FSD406 |
| 315 | FSD407 |
| 316 | FSD408 |
| 317 | FSD409 |
| 318 | FSD410 |
| 319 | FSD411 |
| 320 | FSD412 |
| 321 | FSD413 |
| 322 | FSD414 |
| 323 | FSD415 |
| 324 | FSD416 |
| 325 | FSD417 |
| 326 | FSD418 |
| 327 | FSD419 |
| 328 | FSD421 |
| 329 | FSD422 |
| 330 | FSD423 |
| 331 | FSD424 |
| 332 | FSD425 |
| 333 | FSD426 |
| 334 | FSD427 |
| 335 | FSD428 |
| 336 | FSD429 |
| 337 | FSD430 |
| 338 | FSD431 |
| 339 | FSD432 |
| 340 | FSD433 |
| 341 | FSD434 |
| 342 | FSD435 |
| 343 | FSD436 |
| 344 | FSD438 |

*Peptide names changed from those used in CA 3,040,645.

### DETAILED DESCRIPTION

In some aspects, described herein are methods for non-proteinaceous and/or proteinaceous cargo transduction. The methods generally comprise contacting target eukaryotic cells with a non-proteinaceous and/or proteinaceous cargo and a concentration of a synthetic peptide shuttle agent sufficient to increase the transduction efficiency of the cargo, as compared to in the absence of the synthetic peptide shuttle agent. Also described herein are versatile synthetic peptide shuttle agents having dual transduction activity for both proteinaceous and non-proteinaceous cargoes, as well as the use of PI or other membrane-impermeable 10 fluorescent DNA intercalating agent as a “surrogate” cargo for selecting synthetic peptide shuttle agents having such dual transduction activity.

### Non-Proteinaceous Cargoes

In some embodiments, the non-proteinaceous cargo may be a compound (e.g., organic compound) having a molecular weight of less than 10 000, 9000, 8000, 7000, 6000, 5000, 4000, 3000, 2000, or 1000 Da. In some embodiments, the non-proteinaceous cargo may be a compound (e.g., organic compound) having a molecular weight of between 50 to 5000, 50 to 4000, 50 to 3000, 50 to 2000, or 50 to 1000 Da. In some embodiments, the non-proteinaceous cargo may be a small molecule, such as a small molecule drug that binds to an intracellular biological or therapeutic target. In some embodiments, the non-proteinaceous cargo is not a biopolymer, such as a polynucleotide or a polysaccharide, particularly a biopolymer having a uniform negative charge such as a polynucleotide greater than 50, 60, 70, 80, 90, 100, 150, or 200 nucleotides in length. In some embodiments, the non-proteinaceous cargo may have a cationic net charge in aqueous solution. In some embodiments, the non-proteinaceous cargo is not covalently bound to (i.e., is independent from) the synthetic peptide shuttle agent (e.g., at the moment of transduction).

In some embodiments, the non-proteinaceous cargo may be a cargo that is cell membrane-impermeable or that has low membrane permeability (e.g., due to the physicochemical properties of the cargo precluding it from freely diffusing across the cell membrane), wherein the peptide shuttle agents described herein facilitate or increase its intracellular delivery and/or access to the cytosol. In some embodiments, the non-proteinaceous cargo may be a cargo that is cell membrane-permeable, wherein peptide shuttle agents described herein nevertheless increase its intracellular delivery and/or access to the cytosol. In some embodiments, peptide shuttle agents described herein may reduce the amount or concentration of the cargo that is required to be administered to achieve its intended biological effect, as compared to administration of the cargo alone.

In some embodiments, the non-proteinaceous cargo to be transduced may be a drug for treating any disease or condition having an intracellular biological or therapeutic target. In some embodiments, the non-proteinaceous cargo may be a drug for treating cancer (e.g., skin cancer, basal cell carcinoma, nevoid basal cell carcinoma syndrome), inflammation or an inflammation-related disease (e.g., psoriasis, atopic dermatitis, ulcerative colitis, urticaria, dry eye disease, dry or wet age-related macular degeneration, digital ulcers, actinic keratosis, idiopathic pulmonary fibrosis), pain (e.g., chronic or acute), or a disease affecting the lungs (e.g., cystic fibrosis, asthma, chronic obstructive pulmonary disease (COPD), or idiopathic pulmonary fibrosis).

In particular embodiments, the non-proteinaceous cargo to be transduced may be or comprise a HedgeHog inhibitor (e.g., itraconazole, posaconazole, arsenic trioxide (ATO), Gant61, PF-4708671, HPI-1, HPI-4). In particular embodiments, the non-proteinaceous cargo to be transduced may be or comprise a pain inhibitor, such as a voltage-gated sodium (Nav) channel inhibitor (e.g., QX-314). In particular embodiments, the non-proteinaceous cargo to be transduced may be or comprise an inhibitor of inflammation, such as an inhibitor of a pathway leading to production of inflammatory cytokines (e.g., an NF-kappa B pathway inhibitor).

In some embodiments, the shuttle agents described herein may possess the ability to transduce both non-proteinaceous and proteinaceous cargoes to the cytosol of target eukaryotic cells.

### Rational Design Parameters and Peptide Shuttle Agents

In some aspects, the shuttle agents described herein may be a peptide having transduction activity for proteinaceous cargoes, non-proteinaceous cargoes, or both proteinaceous and non-proteinaceous cargoes in target eukaryotic cells. In some embodiments, the shuttle agents described herein preferably satisfy one or more of the following fifteen rational design parameters.
(1) In some embodiments, the shuttle agent is a peptide at least 17, 18, 19, or 20 amino acids in length. For example, the peptide may comprise a minimum length of 17, 18, 19, 20, 21, 22, 23, 24, 25, 26, 27, 28, 29, or 30 amino acid residues, and a maximum length of 35, 36, 37, 38, 39, 40, 41, 42, 43, 44, 45, 46, 47, 48, 49, 50, 51, 52, 53, 54, 55, 60, 65, 70, 75, 80, 85, 90, 95, 100, 105, 110, 115, 120, 125, 130, 135, 140, 145, or 150 amino acid residues. In some embodiments, shorter peptides (e.g., in the 17-50 or 20-50 amino acid range) may be particularly advantageous because they may be more easily synthesized and purified by chemical synthesis approaches, which may be more suitable for clinical use (as opposed to recombinant proteins that must be purified from cellular expression systems). While numbers and ranges in the present description are often listed as multiples of 5, the present description should not be so limited. For example, the maximum length described herein should be understood as also encompassing a length of 56, 57, 58 . . . 61, 62, etc., in the present description, and that their non-listing herein is only for the sake of brevity. The same reasoning applies to the % of identities listed herein.
(2) In some embodiments, the peptide shuttle agent comprises an amphipathic alpha-helical motif. As used herein, the expression “alpha-helical motif” or “alpha-helix”, unless otherwise specified, refers to a right-handed coiled or spiral conformation (helix) having angle of rotation between consecutive amino acids of 100 degrees and/or an alpha-helix having 3.6 residues per turn. As used herein, the expression “comprises an alpha-helical motif” or “an amphipathic alpha-helical motif” and the like, refers to the three-dimensional conformation that a peptide (or segment of a peptide) of the present description is predicted to adopt when in a biological setting based on the peptide's primary amino acid sequence, regardless of whether the peptide actually adopts that conformation when used in cells as a shuttle agent. Furthermore, the peptides of the present description may comprise one or more alpha-helical motifs in different locations of the peptide. For example, the shuttle agent FSD5 in WO/2018/068135 is predicted to adopt an alpha-helix over the entirety of its length (see FIG. 49C of WO/2018/068135), while the shuttle agent FSD18 of WO/2018/068135 is predicted to comprise two separate alpha-helices towards the N and C terminal regions of the peptide (see FIG. 49D of WO/2018/068135). In some embodiments, the shuttle agents of the present description are not predicted to comprise a beta-sheet motif, for example as shown in FIGS. 49E and 49F of WO/2018/068135. Methods of predicting the presence of alpha-helices and beta-sheets in proteins and peptides are well known in the art. For example, one such method is based on 3D modeling using PEP-FOLD™, an online resource for de novo peptide structure prediction (world wide web site bioserv.rpbs.univ-paris-diderot.fr/services/PEP-FOLD/) (Lamiable et al., 2016; Shen et al., 2014; Thévenet et al., 2012). Other methods of predicting the presence of alpha-helices in peptides and protein are known and readily available to the skilled person.

As used herein, the expression “amphipathic” refers to a peptide that possesses both hydrophobic and hydrophilic elements (e.g., based on the side chains of the amino acids that comprise the peptide). For example, the expression “amphipathic alpha helix” or “amphipathic alpha-helical motif” refers to a peptide predicted to adopt an alpha-helical motif having a non-polar hydrophobic face and a polar hydrophilic face, based on the properties of the side chains of the amino acids that form the helix.
(3) In some embodiments, peptide shuttle agents of the present description comprise an amphipathic alpha-helical motif having a positively-charged hydrophilic outer face, such as one that is rich in R and/or K residues. As used herein, the expression “positively-charged hydrophilic outer face” refers to the presence of at least three lysine (K) and/or arginine (R) residues clustered to one side of the amphipathic alpha-helical motif, based on alpha-helical wheel projection (e.g., see FIG. 49A, left panel of WO/2018/068135). Such helical wheel projections may be prepared using a variety of programs, such as the online helical wheel projection tool available at: world wide web site rzlab.ucr.edu/scripts/wheel/wheel.cgi. In some embodiments, the amphipathic alpha-helical motif may comprise a positively-charged hydrophilic outer face that comprises: (a) at least two, three, or four adjacent positively-charged K and/or R residues upon helical wheel projection; and/or (b) a segment of six adjacent residues comprising three to five K and/or R residues upon helical wheel projection, based on an alpha helix having angle of rotation between consecutive amino acids of 100 degrees and/or an alpha-helix having 3.6 residues per turn.

In some embodiments, peptide shuttle agents of the present description comprise an amphipathic alpha-helical motif comprising a hydrophobic outer face, the hydrophobic outer face comprising: (a) at least two adjacent L residues upon helical wheel projection; and/or (b) a segment of ten adjacent residues comprising at least five hydrophobic residues selected from: L, I, F, V, W, and M, upon helical wheel projection, based on an alpha helix having angle of rotation between consecutive amino acids of 100 degrees and/or an alpha-helix having 3.6 residues per turn.
(4) In some embodiments, peptide shuttle agents of the present description comprise an amphipathic alpha-helical motif having a highly hydrophobic core composed of spatially adjacent highly hydrophobic residues (e.g., L, I, F, V, W, and/or M). In some embodiments, the highly hydrophobic core may consist of spatially adjacent L, I, F, V, W, and/or M amino acids representing 12 to 50% of the amino acids of the peptide, calculated while excluding any histidine-rich domains (see below), based on an open cylindrical representation of the alpha-helix having 3.6 residues per turn, as shown for example in FIG. 49A, right panel of WO/2018/068135. In some embodiments, the highly hydrophobic core may consist of spatially adjacent L, I, F, V, W, and/or M amino acids representing from 12.5%, 13%, 13.5%, 14%, 14.5%, 15%, 15.5%, 16%, 16.5%, 17%, 17.5%, 18%, 18.5%, 19%, 19.5%, or 20%, to 25%, 30%, 35%, 40%, or 45% of the amino acids of the peptide. More particularly, highly hydrophobic core parameter may be calculated by first arranging the amino acids of the peptide in an opened cylindrical representation, and then delineating an area of contiguous highly hydrophobic residues (L, I, F, V, W, M), as shown in FIG. 49A, right panel of WO/2018/068135. The number of highly hydrophobic residues comprised in this delineated highly hydrophobic core is then divided by the total amino acid length of the peptide, excluding any histidine-rich domains (e.g., N- and/or C-terminal histidine-rich domains). For example, for the peptide shown in FIG. 49A of WO/2018/068135, there are 8 residues in the delineated highly hydrophobic core, and 25 total residues in the peptide (excluding the terminal 12 histidines). Thus, the highly hydrophobic core is 32% (8/25).
(5) Hydrophobic moment relates to a measure of the amphiphilicity of a helix, peptide, or part thereof, calculated from the vector sum of the hydrophobicities of the side chains of the amino acids (Eisenberg et al., 1982). An online tool for calculating the hydrophobic moment of a polypeptide is available from: world wide web site rzlab.ucr.edu/scripts/wheel/wheel.cgi. A high hydrophobic moment indicates strong amphiphilicity, while a low hydrophobic moment indicates poor amphiphilicity. In some embodiments, peptide shuttle agents of the present description may consist of or comprise a peptide or alpha-helical domain having have a hydrophobic moment (μ) of 3.5 to 11. In some embodiments, the shuttle agent may be a peptide comprising an amphipathic alpha-helical motif having a hydrophobic moment between a lower limit of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, 10.4, 10.5, 10.6, 10.7, 10.8, 10.9, or 11.0. In some embodiments, the shuttle agent may be a peptide having a hydrophobic moment between a lower limit of 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, 10.4, or 10.5. In some embodiments, the hydrophobic moment is calculated excluding any histidine-rich domains that may be present in the peptide.
(6) In some embodiments, peptide shuttle agents of the present description may have a predicted net charge of at least +4 at physiological pH, calculated from the side chains of K, R, D, and E residues. For example, the net charge of the peptide may be at least +5, +6, +7, at least +8, at least +9, at least +10, at least +11, at least +12, at least +13, at least +14, or at least +15 at physiological pH. These positive charges are generally conferred by the greater presence of positively-charged lysine and/or arginine residues, as opposed to negatively charged aspartate and/or glutamate residues.
(7) In some embodiments, peptide shuttle agents of the present description may have a predicted isoelectric point (pI) of 8 to 13, preferably from 10 to 13. Programs and methods for calculating and/or measuring the isoelectric point of a peptide or protein are known in the art. For example, pI may be calculated using the Prot Param software available at: world wide web site web.expasy.org/protparam/.
(8) In some embodiments, peptide shuttle agents of the present description may be composed of 35 to 65% of hydrophobic residues (A, C, G, I, L, M, F, P, W, Y, V). In particular embodiments, the peptide shuttle agents may be composed of 36% to 64%, 37% to 63%, 38% to 62%, 39% to 61%, or 40% to 60% of any combination of the amino acids: A, C, G, I, L, M, F, P, W, Y, and V.
(9) In some embodiments, peptide shuttle agents of the present description may be composed of 0 to 30% of neutral hydrophilic residues (N, Q, S, T). In particular embodiments, the peptide shuttle agents may be composed of 1% to 29%, 2% to 28%, 3% to 27%, 4% to 26%, 5% to 25%, 6% to 24%, 7% to 23%, 8% to 22%, 9% to 21%, or 10% to 20% of any combination of the amino acids: N, Q, S, and T.
(10) In some embodiments, peptide shuttle agents of the present description may be composed of 35 to 85% of the amino acids A, L, K and/or R. In particular embodiments, the peptide shuttle agents may be composed of 36% to 80%, 37% to 75%, 38% to 70%, 39% to 65%, or 40% to 60% of any combination of the amino acids: A, L, K, or R.
(11) In some embodiments, peptide shuttle agents of the present description may be composed of 15 to 45% of the amino acids A and/or L, provided there being at least 5% of L in the peptide. In particular embodiments, the peptide shuttle agents may be composed of 15% to 40%, 20% to 40%, 20 to 35%, or 20 to 30% of any combination of the amino acids: A and L, provided there being at least 5% of L in the peptide.
(12) In some embodiments, peptide shuttle agents of the present description may be composed of 20 to 45% of the amino acids K and/or R. In particular embodiments, the peptide shuttle agents may be composed of 20% to 40%, 20 to 35%, or 20 to 30% of any combination of the amino acids: K and R.
(13) In some embodiments, peptide shuttle agents of the present description may be composed of 0 to 10% of the amino acids D and/or E. In particular embodiments, the peptide shuttle agents may be composed of 5 to 10% of any combination of the amino acids: D and E.
(14) In some embodiments, the absolute difference between the percentage of A and/or L and the percentage of K and/or R in the peptide shuttle agent may be less than or equal to 10%. In particular embodiments, the absolute difference between the percentage of A and/or L and the percentage of K and/or R in the peptide shuttle agent may be less than or equal to 9%, 8%, 7%, 6%, or 5%.
(15) In some embodiments, peptide shuttle agents of the present description may be composed of 10% to 45% of the amino acids Q, Y, W, P, I, S, G, V, F, E, D, C, M, N, T, or H (i.e., not A, L, K, or R). In particular embodiments, the peptide shuttle agents may be composed of 15 to 40%, 20% to 35%, or 20% to 30% of any combination of the amino acids: Q, Y, W, P, I, S, G, V, F, E, D, C, M, N, T, and H.

In some embodiments, peptide shuttle agents of the present description respect at least one, at least two, at least three, at least four, at least five, at least six, at least seven, at least eight, at least nine, at least ten, at least eleven, at least twelve, at leave thirteen, at least fourteen, or all of parameters (1) to (15) described herein. In particular embodiments, peptide shuttle agents of the present description respect all of parameters (1) to (3), and at least six, at least seven, at least eight, at least nine, at least ten, at least eleven, or all of parameters (4) to (15) described herein.

In some embodiments, where a peptide shuttle agent of the present description comprises only one histidine-rich domain, the residues of the one histidine-rich domain may be included in the calculation/assessment of parameters (1) to (15) described herein. In some embodiments, where a peptide shuttle agent of the present description comprises more than one histidine-rich domain, only the residues of one of the histidine-rich domains may be included in the calculation/assessment of parameters (1) to (15) described herein. For example, where a peptide shuttle agent of the present description comprises two histidine-rich domains: a first histidine-rich domain towards the N terminus, and a second histidine-rich domain towards the C terminus, only the first histidine-rich domain may be included in the calculation/assessment of parameters (1) to (15) described herein.

In some embodiments, a machine-learning or computer-assisted design approach may be implemented to generate peptides that respect one or more of parameters (1) to (15) described herein. Some parameters, such as parameters (1) and (5)-(15), may be more amenable to implementation in a computer-assisted design approach, while structural parameters, such as parameters (2), (3) and (4), may be more amenable to a manual design approach. Thus, in some embodiments, peptides that respect one or more of parameters (1) to (15) may be generated by combining computer-assisted and manual design approaches. For example, multiple sequence alignment analyses of a plurality of peptides shown herein (and others) to function as effective shuttle agents revealed the presence of some consensus sequences—i.e., commonly found patterns of alternance of hydrophobic, cationic, hydrophilic, alanine and glycine amino acids. The presence of these consensus sequences are likely to give rise to structural parameters (2), (3) and (4) being respected (i.e., amphipathic alpha-helix formation, a positively-charged face, and a highly hydrophobic core of 12%-50%). Thus, these and other consensus sequences may be employed in machine-learning and/or computer-assisted design approaches to generate peptides that respect one or of parameters (1)-(15).

Accordingly, in some embodiments, peptide shuttle agents described herein may comprise or consist of the amino acid sequence of:

(a) (Formula 1)
[X1]-[X2]-[linker]-[X3]-[X4];
(b) (Formula 2)
[X1]-[X2]-[linker]-[X4]-[X3];
(c) (Formula 3)
[X2]-[X1]-[linker]-[X3]-[X4];
(d) (Formula 4)
[X2]-[X1]-[linker]-[X4]-[X3];
(e) (Formula 5)
[X3]-[X4]-[linker]-[X1]-[X2];
(f) (Formula 6)
[X3]-[X4]-[linker]-[X2]-[X1];
(g) (Formula 7)
[X4]-[X3]-[linker]-[X1]-[X2];
or
(h) (Formula 8)
[X4]-[X3]-[linker]-[X2]-[X1],

wherein:
[X1] is selected from: 2[Φ]-1[+]-2[Φ]-1[ζ]-1[+]-; 2[Φ]-1[+]-2[Φ]-2[+]-; 1[+]-1[Φ]-1[+]-2[Φ]-1[ζ]-1[+]-; and 1[+]-1[Φ]-1[+]-2[Φ]-2[+]-;
[X2] is selected from: -2[Φ]-1[+]-2[Φ]-2[ζ]-; -2[Φ]-1[+]-2[Φ]-2[+]-; -2[Φ]-1[+]-2[Φ]-1[+]-1[ζ]-; -2[Φ]-1[+]-2[Φ]-1[ζ]-1[+]-; -2[Φ]-2[+]-1[Φ]-2[+]-; -2[Φ]-2[+]-1[Φ]-2[ζ]-; -2[Φ]-2[+]-1[Φ]-1[+]-1[ζ]-; and -2[(I]-2[+]-1[Φ]-1[ζ]-1[+]-;
[X3] is selected from: -4[+]-A-; -3[+]-G-A-; -3[+]-A-A-; -2[+]-1[Φ]-1[+]-A-; -2[+]-1[Φ]-G-A-; -2[+]-1[Φ]-A-A-; or -2[+]-A-1[+]-A; -2[+]-A-G-A; -2[+]-A-A-A-; -1[Φ]-3[+]-A-; -1[Φ]-2[+]-G-A-; -1[Φ]-2[+]-A-A-; -1[Φ]-1[+]-1[Φ]-1[+]-A; -1[Φ]-1[+]-1[Φ]-G-A; -1[Φ]-1[+]-1[Φ]-A-A; -1[Φ]-1[+]-A-1[+]-A; -1[Φ]-1[+]-A-G-A; -1[ζ]-1[+]-A-A-A; -A-1[+]-A-1[+]-A; -A-1[+]-A-G-A (SEQ ID NO: 345); and -A-1[+]-A-A-A (SEQ ID NO: 346);
[X4] is selected from: -1[ζ]-2A-1[+]-A; -1[ζ]-2A-2[+]; -1[+]-2A-1[+]-A; -1[ζ]-2A-1[+]-1[ζ]-A-1[+]; -1[ζ]-A-1[ζ]-A-1[+]; -2[+]-A-2[+]; -2[+]-A-1[+]-A; -2[+]-A-1[+]-1[ζ]-A-1[+]; -2[+]-1[ζ]-A-1[+]; -1[+]-1[ζ]-A-1[+]-A; -1[+]-1[ζ]-A-2[+]; -1[+]-1[ζ]-A-1[+]-1[ζ]-A-1[+]; -1[+]-2[ζ]- A-1[+]; -1[+]-2[ζ]-2[+]; -1[+]-2[ζ]-1[+]-A; -1[+]-2[ζ]-1[+]-1[ζ]-A-1[+]; -1[+]-2[ζ]-1[ζ]-A-1[+]; -3[ζ]-2[+]; -3[ζ]-1[+]-A; -3-[ζ]1-[+]-1[ζ]-2A-1[+]-A; -1[ζ]-2A-2[+]; -1[ζ]-2A-1[+]-1[ζ]-A-1[+]; -2[+]-A-1[+]-A; -2[+]-1[ζ]-1[+]-A; -1[+]-1[ζ]-A-1[+]-A; -1[+]-2A-1[+]-1[ζ]-A-1[+]; and -1[ζ]-A-1[ζ]-A-1[+]; and
[linker] is selected from: -Gn- (SEQ ID NO: 347); -Sn- (SEQ ID NO: 348); -(GnSn)n- (SEQ ID NO: 349); -(GnSn)nGn- (SEQ ID NO: 350); -(GnSn)nSn- (SEQ ID NO: 351); -(GnSn)nGn(GnSn)n- (SEQ ID NO: 352); and -(GnSn)nSn(GnSn)n- (SEQ ID NO: 353);
   wherein: [Φ] is an amino acid which is: Leu, Phe, Trp, Ile, Met, Tyr, or Val, preferably Leu, Phe, Trp, or Ile; [+] is an amino acid which is: Lys or Arg; [ζ] is an amino acid which is: Gln, Asn, Thr, or Ser; A is the amino acid Ala; G is the amino acid Gly; S is the amino acid Ser; and n is an integer from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 1 to 4, or 1 to 3.

In some embodiments, peptide shuttle agents of the present description may comprise or consist of a peptide which is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of any one of SEQ ID NOs: 1 to 50, 58 to 78, 80 to 107, 109 to 139, 141 to 146, 149 to 161, 163 to 169,171,174 to 234, 236 to 240, 242 to 260, 262 to 285, 287 to 294, 296 to 300, 302 to 308, 310, 311, 313 to 324, 326 to 332, 338 to 342, or 344, or to the amino acid sequence of any one of SEQ ID NOs: 104, 105, 107, 108, 110-131, 133-135, 138, 140, 142, 145, 148, 151, 152, 169-242, and 243-10 242 as disclosed in WO/2018/068135, or a functional variant thereof. In some embodiments, peptide shuttle agents of the present description may comprise the amino acid sequence motifs of SEQ ID NOs: 158 and/or 159 of WO/2018/068135, which were found in each of peptides FSD5, FSD16, FSD18, FSD19, FSD20, FSD22, and FSD23. In some embodiments, peptide shuttle agents of the present description may comprise the amino acid sequence motif of SEQ ID NO: 158 of WO/2018/068135 operably linked to the amino acid sequence motif of SEQ ID NO: 159 of WO/2018/068135. As used herein, a “functional variant” refers to a peptide having cargo transduction activity, which differs from the reference peptide by one or more conservative amino acid substitutions. As used herein in the context of functional variants, a “conservative amino acid substitution” is one in which one amino acid residue is replaced with another amino acid residue having a similar side chain. Families of amino acid residues having similar side chains have been well defined in the art, including basic side chains (e.g., lysine, arginine, histidine), acidic side chains (e.g., aspartic acid, glutamic acid), uncharged polar side chains (e.g., glycine, asparagine, glutamine, seine, threonine, tyrosine, cysteine), nonpolar side chains (e.g., alanine, valine, leucine, isoleucine, phenylalanine, methionine, tryptophan, and optionally proline), beta-branched side chains (e.g., threonine, valine, isoleucine) and aromatic side chains (e.g., tyrosine, phenylalanine, tryptophan, histidine).

In some embodiments, peptide shuttle agents of the present description do not comprise one or more of the amino acid sequences of any one of SEQ ID NOs: 57-59, 66-72, or 82-102 of WO/2018/068135. In some embodiments, peptide shuttle agents of the present description do not comprise one or more of the amino acid sequences of any one of SEQ ID NOs: 104, 105, 107, 108, 110-131, 133-135, 138, 140, 142, 145, 148, 151, 152, 169-242, and 243-10 242 as disclosed in WO/2018/068135. Rather, in some embodiments, peptide shuttle agents of the present description may relate to variants of such previously described shuttle agent peptides, wherein the variants are further engineered for improved dual transduction activity (i.e., capable of more robustly transducing proteinaceous and non-proteinaceous cargoes).

In some embodiments, peptide shuttle agents of the present description may have a minimal threshold of transduction efficiency and/or cargo delivery score for a “surrogate” cargo as measured in a eukaryotic cell model system (e.g., an immortalized eukaryotic cell line) or in a model organism. The expression “transduction efficiency” refers to the percentage or proportion of a population of target cells into which a cargo of interest is delivered intracellularly, which can be determined for example by flow cytometry, immunofluorescence microscopy, and other suitable methods may be used to assess cargo transduction efficiency (e.g., as described in WO/2018/068135). In some embodiments, transduction efficiency may be expressed as a percentage of cargo-positive cells. In some embodiments, transduction efficiency may be expressed as a fold-increase (or fold-decrease) over a suitable negative control assessed under identical conditions except for in the absence of cargo and shuttle agent (“no treatment”; NT) or in the absence of shuttle agent (“cargo alone”).

As used herein, the expression “surrogate cargo” refers to any proteinaceous or non-proteinaceous cargo that can be transduced by a shuttle agent having known cargo transduction activity whose level of intracellular delivery and endosomal escape (i.e., cytosolic and/or nuclear delivery) can be readily measured and/or tracked (e.g., via fluorescence or a functional assay), wherein the surrogate cargo is intended to assess the suitability of a given shuttle agent for transducing a cargo of interest (e.g., proteinaceous or non-proteinaceous cargo, such as a therapeutically active cargo binding to an intracellular target) that is different from the surrogate cargo. Examples of suitable surrogate cargoes may include fluorescent cargoes (e.g., PI or other membrane-impermeable fluorescent DNA intercalating agents, GFP, GFP-NLS or other fluorescent proteins, fluorescent dextran, etc.). Non-proteinaceous cargoes such as PI or other membrane-impermeable fluorescent DNA intercalating agents may be particularly advantageous because they are relatively inexpensive and exhibit enhanced fluorescence only after being bound to genomic DNA—a property that makes them particularly suitable to distinguish endosomally-trapped cargo from endosomally-escaped cargo (i.e., cargoes gaining access to the cytosolic/nuclear compartment). As used herein, any suitable model system (e.g., immortalized cell lines, ex vivo cells, model laboratory organisms) may be used to assess shuttle agent transduction activity for the surrogate cargo. Conveniently, a eukaryotic cell line model may be selected as a suitable model system, wherein the cell line is selected to be informative for assessing transduction activity in the target eukaryotic cells that will ultimately be transduced. Indeed, a plurality of cell cultures and model organisms are commercially available as model system to study various diseases.

In some embodiments, peptide shuttle agents of the present description increase the transduction efficiency of propidium iodide or other membrane-impermeable fluorescent DNA intercalating agent in a suitable eukaryotic cell model system (e.g., in HeLa or other suitable immortalized cell line). In some embodiments, peptide shuttle agents of the present description increase the transduction efficiency of propidium iodide or other membrane-impermeable fluorescent DNA intercalating agent by at least 3, 3.5, 4, 4.5, 5, 5.5, 6, 6.5, 7, 7.5, 8, 8.5, 9, 9.5, or 10-fold over a corresponding negative control lacking said shuttle agent (“cargo alone”), in HeLa cells or other suitable eukaryotic cell line model for assessing cargo transduction in the target eukaryotic cells of interest. In some embodiments, peptide shuttle agents of the present description enable a transduction efficiency of propidium iodide or other membrane-impermeable fluorescent DNA intercalating agent of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) in HeLa cells or other suitable eukaryotic cell line model for assessing cargo transduction in the target eukaryotic cells of interest.

In some embodiments, peptide shuttle agents of the present description increase the transduction efficiency of GFP-NLS or other suitable proteinaceous surrogate cargo in a suitable eukaryotic cell model system (e.g., in HeLa or other suitable immortalized cell line). In some embodiments, peptide shuttle agents of the present description increase the transduction efficiency of GFP-NLS or other suitable proteinaceous surrogate cargo by at least 3, 3.5, 4, 4.5, 5, 5.5, 6, 6.5, 7, 7.5, 8, 8.5, 9, 9.5, 10, 15, 20, 25, 30, 35, 40, 45 or 50-fold over a corresponding negative control lacking said shuttle agent (“cargo alone”), in HeLa cells or other suitable eukaryotic cell line model for assessing cargo transduction in the target eukaryotic cells of interest. In some embodiments, peptide shuttle agents of the present description enable a transduction efficiency of GFP-NLS or other suitable proteinaceous surrogate cargo of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) in HeLa cells or other suitable eukaryotic cell line model for assessing cargo transduction in the target eukaryotic cells of interest.

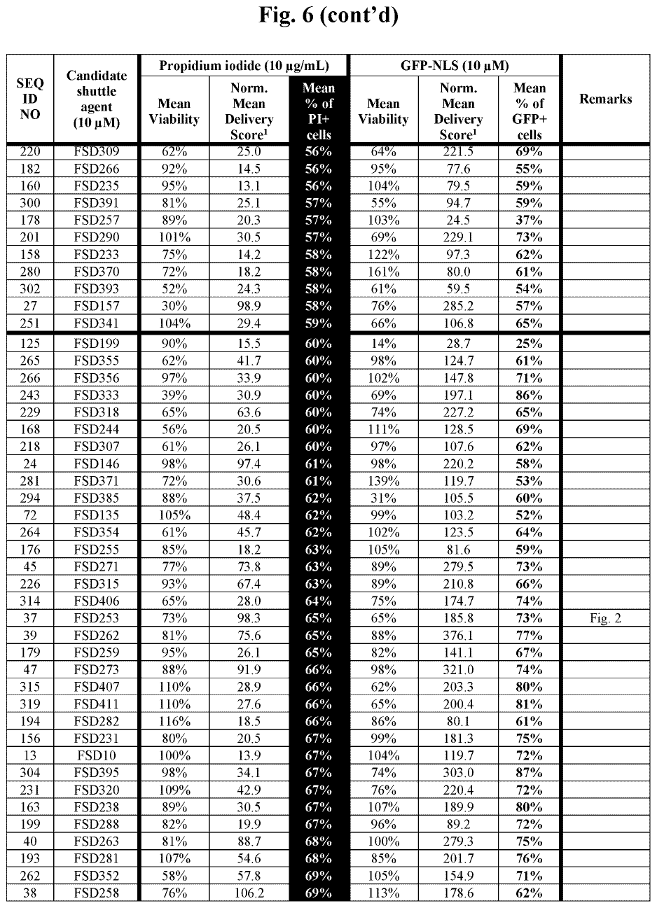

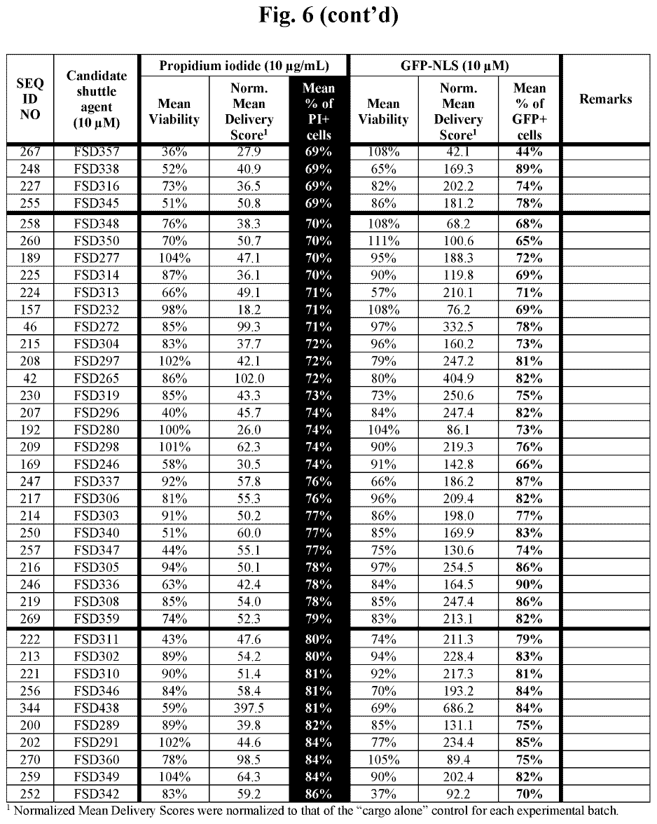

In some embodiments, peptide shuttle agents of the present description may comprise or consist of the shuttle agents listed in FIG. 6 having a mean PI transduction efficiency of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60%. In some embodiments, peptide shuttle agents of the present description may comprise or consist of a shuttle agent listed in FIG. 6 having a normalized mean PI delivery score of at least 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, or 60.

In some embodiments, peptide shuttle agents of the present description may comprise or consist of the shuttle agents listed in FIG. 6 having a mean GFP-NLS transduction efficiency of at least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60%. In some embodiments, peptide shuttle agents of the present description may comprise or consist of the shuttle agents listed in FIG. 6 having a normalized mean GFP-NLS delivery score of at least 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 65, 70, 75, 80, 85, 90, 95, 100, 105, 110, 115, 120, 125, 130, 135, 140, 145, 150, 155, 160, 165, 170, 175, 180, 185, 190, 195, or 200.

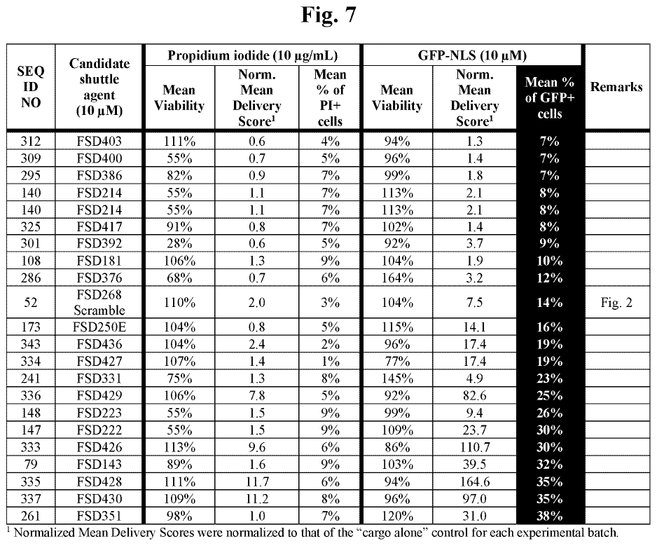

In some embodiments, peptide shuttle agents of the present description may comprise or consist of the shuttle agents listed in FIG. 7 having a mean GFP-NLS transduction efficiency of at least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%, or a normalized mean GFP-NLS delivery score of at least 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30.

In some embodiments, the shuttle agents of the present description may comprise shuttle agent variants that differ from the shuttle agents defined herein by no more than 1, 2, 3, 4, 5, 6, 7, 8, 9, or 10 amino acids. Preferably, linker domains (e.g., flexible serine/glycine-rich linker domains) are excluded from the differing amino acid consideration, as the lengths and/or amino acid composition of the linker domains may greatly vary without affecting transduction activity. In some embodiments, peptide shuttle agents of the present description may comprise or consist of an amino acid sequence that differs from any one of the shuttle agents described herein by only conservative amino acid substitutions (e.g., by no more than no more than 1, 2, 3, 4, 5, 6, 7, 8, 9, or 10 conservative amino acid substitutions, preferably excluding any linker domains), wherein shuttle agent: increases the transduction efficiency of propidium iodide or other membrane-impermeable fluorescent DNA intercalating agent by at least 3, 3.5, 4, 4.5, 5, 5.5, 6, 6.5, 7, 7.5, 8, 8.5, 9, 9.5, or 10-fold over a corresponding negative control lacking said shuttle agent; and/or enables a transduction efficiency of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of propidium iodide or other membrane-impermeable fluorescent DNA intercalating agent, in a eukaryotic cell line model (e.g., HeLa) suitable for assessing cargo transduction in said target eukaryotic cells. In some embodiments, each conservative amino acid substitution is selected from an amino acid within the same amino acid class, the amino acid class being: Aliphatic: G, A, V, L, and I; Hydroxyl or sulfur/selenium-containing: S, C, U, T, and M; Aromatic: F, Y, and W; Basic: H, K, and R; Acidic and their amides: D, E, N, and Q.

### Chemical Modifications and Synthetic Amino Acids

In some embodiments, shuttle agents of the present description may comprise oligomers (e.g., dimers, trimers, etc.) of peptides described herein. Such oligomers may be constructed by covalently binding the same or different types of shuttle agent monomers (e.g., using disulfide bridges to link cysteine residues introduced into the monomer sequences). In some embodiments, shuttle agents of the present description may comprise an N-terminal and/or a C-terminal cysteine residue.

In some embodiments, shuttle agents of the present description may comprise or consist of a cyclic peptide. In some embodiments, the cyclic peptide may be formed via a covalent link between a first residue positioned towards the N terminus of the shuttle agent and a second residue positioned towards the C terminus of the shuttle agent. In some embodiments, the first and second residues are flanking residues positioned at the N and the C termini of the shuttle agent. In some embodiments, the first and second residues may be linked via an amide linkage to form the cyclic peptide. In some embodiments, the cyclic peptide may be formed by a disulfide bond between two cysteine residues within the shuttle agent, wherein the two cysteine residues are positioned towards the N and C termini of the shuttle agent. In some embodiments, the shuttle agent may comprise, or be engineered to comprise, flanking cysteine residues at the N and C termini, which are linked via a disulfide bond to form the cyclic peptide. In some embodiments, the cyclic shuttle agents described herein may be more resistant to degradation (e.g., by proteases) and/or may have a longer half-life than a corresponding linear peptide.

In some embodiments, the shuttle agents of the present description may comprise one or more D-amino acids. In some embodiments, the shuttle agents of the present description may comprise a D-amino acid at the N and/or C terminus of the shuttle agent. In some embodiments, the shuttle agents maybe comprised entirely of D-amino acids. In some embodiments, the shuttle agents described herein having one or more D-amino acids may be more resistant to degradation (e.g., by proteases) and/or may have a longer half-life than a corresponding peptide comprised of only L-amino acids.

In some embodiments, the shuttle agents of the present description may comprise a chemical modification to one or more amino acids, wherein the chemical modification does not destroy the transduction activity of the synthetic peptide shuttle agent. As used herein in this context, the term “destroy” means that the chemical modification irreversibly abolishes the cargo transduction activity of a peptide shuttle agent described herein. Chemical modifications that may transiently inhibit, attenuate, or delay the cargo transduction activity of a peptide shuttle agent described herein may be included in the chemical modifications to the shuttle agents of the present description. In some embodiments, the chemical modification to any one of the shuttle agents described herein may be at the N and/or C terminus of the shuttle agent. Examples of chemical modifications include the addition of an acetyl group (e.g., an N-terminal acetyl group), a cysteamide group (e.g., a C-terminal cysteamide group), or a fatty acid (e.g., C4-C16, C6-C14, C6-C12, C6-C8, or C8 fatty acid, preferably being N-terminal).

In some embodiments, the shuttle agents of the present description comprise shuttle agent variants having transduction activity for proteinaceous and/or non-proteinaceous cargoes in target eukaryotic cells, the variants being identical to any shuttle agent of the present description, except having at least one amino acid being replaced with a corresponding synthetic amino acid or amino acid analog having a side chain of similar physiochemical properties (e.g., structure, hydrophobicity, or charge) as the amino acid being replaced. In some embodiments, the synthetic amino acid replacement:
(a) replaces a basic amino acids with any one of α-aminoglycine, α,γ-diaminobutyric acid, ornithine, α,β-diaminopropionic acid, 2,6-diamino-4-hexynoic acid, β-(1-piperazinyl)-alanine, 4,5-dehydro-lysine, δ-hydroxylysine, ω,ω-dimethylarginine, homoarginine, ω,ω′-dimethylarginine, ω-methylarginine, β-(2-quinolyl)-alanine, 4-aminopiperidine-4-carboxylic acid, α-methylhistidine, 2,5-diiodohistidine, 1-methylhistidine, 3-methylhistidine, spinacine, 4-aminophenylalanine, 3-aminotyrosine, β-(2-pyridyl)-alanine, or β-(3-pyridyl)-alanine;
(b) replaces a non-polar (hydrophobic) amino acid with any one of dehydro-alanine, β-fluoroalanine, β-chloroalanine, β-lodoalanine, α-aminobutyric acid, α-aminoisobutyric acid, β-cyclopropylalanine, azetidine-2-carboxylic acid, α-allylglycine, propargylglycine, tert-butylalanine, β-(2-thiazolyl)-alanine, thiaproline, 3,4-dehydroproline, tert-butylglycine, β-cyclopentylalanine, β-cyclohexylalanine, α-methylproline, norvaline, α-methylvaline, penicillamine, β, β-dicyclohexylalanine, 4-fluoroproline, 1-aminocyclopentanecarboxylic acid, pipecolic acid, 4,5-dehydroleucine, allo-isoleucine, norleucine, α-methylleucine, cyclohexylglycine, cis-octahydroindole-2-carboxylic acid, β-(2-thienyl)-alanine, phenylglycine, α-methylphenylalanine, homophenylalanine, 1,2,3,4-tetrahydroisoquinoline-3-carboxylic acid, β-(3-benzothienyl)-alanine, 4-nitrophenylalanine, 4-bromophenylalanine, 4-tert-butylphenylalanine, α-methyltryptophan, β-(2-naphthyl)-alanine, β-(1-naphthyl)-alanine, 4-iodophenylalanine, 3-fluorophenylalanine, 4-fluorophenylalanine, 4-methyltryptophan, 4-chlorophenylalanine, 3,4-dichloro-phenylalanine, 2,6-difluoro-phenylalanine, n-in-methyltryptophan, 1,2,3,4-tetrahydronorharman-3-carboxylic acid, β,β-diphenylalanine, 4-methylphenylalanine, 4-phenylphenylalanine, 2,3,4,5,6-pentafluoro-phenylalanine, or 4-benzoylphenylalanine;
(c) replaces a polar, uncharged amino acid with any one of β-cyanoalanine, β-ureidoalanine, homocysteine, allo-threonine, pyroglutamic acid, 2-oxothiazolidine-4-carboxylic acid, citrulline, thiocitrulline, homocitrulline, hydroxyproline, 3,4-dihydroxyphenylalanine, β-(1,2,4-triazol-1-yl)-alanine, 2-mercaptohistidine, β-(3,4-dihydroxyphenyl)-serine, β-(2-thienyl)-serine, 4-azidophenylalanine, 4-cyanophenylalanine, 3-hydroxymethyltyrosine, 3-iodotyrosine, 3-nitrotyrosine, 3,5-dinitrotyrosine, 3,5-dibromotyrosine, 3,5-diiodotyrosine, 7-hydroxy-1,2,3,4-tetrahydroiso-quinoline-3-carboxylic acid, 5-hydroxytryptophan, thyronine, β-(7-methoxycoumarin-4-yl)-alanine, or 4-(7-hydroxy-4-coumarinyl)-aminobutyric acid; and/or
(d) replaces an acidic amino acid with any one of: γ-hydroxyglutamic acid, γ-methyleneglutamic acid, γ-carboxyglutamic acid, α-aminoadipic acid, 2-aminoheptanedioic acid, α-aminosuberic acid, 4-carboxyphenylalanine, cysteic acid, 4-phosphonophenylalanine, or 4-sulfomethylphenylalanine.

### Histidine-Rich Domains

In some embodiments, peptide shuttle agents of the present description may further comprise one or more histidine-rich domains. In some embodiments, the histidine-rich domain may be a stretch of at least 2, at least 3, at least 4, at least 5, or at least 6 amino acids comprising at least 30%, at least 35%, at least 40%, at least 45%, at least 50%, at least 55%, at least 60%, at least 65%, at least 70%, at least 75%, at least 80%, at least 85%, or at least 90% histidine residues. In some embodiments, the histidine-rich domain may comprise at least 2, at least 3, at least 4 at least 5, at least 6, at least 7, at least 8, or at least 9 consecutive histidine residues (SEQ ID NO: 368). Without being bound by theory, the histidine-rich domain in the shuttle agent may act as a proton sponge in the endosome through protonation of their imidazole groups under acidic conditions of the endosomes, providing another mechanism of endosomal membrane destabilization and thus further facilitating the ability of endosomally-trapped cargoes to gain access to the cytosol. In some embodiments, the histidine-rich domain may be located at or towards the N and/or C terminus of the peptide shuttle agent.

### Linkers

In some embodiments, peptide shuttle agents of the present description may comprise one or more suitable linkers (e.g., flexible polypeptide linkers). In some embodiments, such linkers may separate two or more amphipathic alpha-helical motifs (e.g., see the shuttle agent FSD18 in FIG. 49D of WO/2018/068135). In some embodiments, linkers can be used to separate two more domains (CPDs, ELDs, or histidine-rich domains) from one another. In some embodiments, linkers may be formed by adding sequences of small hydrophobic amino acids without rotatory potential (such as glycine) and polar serine residues that confer stability and flexibility. Linkers may be soft and allow the domains of the shuttle agents to move. In some embodiments, prolines may be avoided since they can add significant conformational rigidity. In some embodiments, the linkers may be serine/glycine-rich linkers (e.g., GS, GGS, GGSGGGS (SEQ ID NO.:354), GGSGGGSGGGS (SEQ ID NO.:355), or the like). In some embodiments, the use shuttle agents comprising a suitable linker may be advantageous for delivering a cargo to suspension cells, rather than to adherent cells. In some embodiments, the linker may comprise or consist of: -Gn- (SEQ ID NO: 356); -Sn- (SEQ ID NO: 357); -(GnSn)n- (SEQ ID NO: 358); -(GnSn)nGn- (SEQ ID NO: 359); -(GnSn)nSn- (SEQ ID NO: 360); -(GnSn)nGn(GnSn)n- (SEQ ID NO: 362); or -(GnSn)nSn(GnSn)n- (SEQ ID NO: 363), wherein G is the amino acid Gly; S is the amino acid Ser; and n is an integer from 1 to 5.

### Domain-Based Peptide Shuttle Agents

In some aspects, the shuttle agents described herein may be a shuttle agent as described in WO/2016/161516, comprising an endosome leakage domain (ELD) operably linked to a cell penetrating domain (CPD).

### Endosome Leakage Domains (ELDs)

In some aspects, peptide shuttle agents of the present description may comprise an endosome leakage domain (ELD) for facilitating endosome escape and access to the cytoplasmic compartment. As used herein, the expression “endosome leakage domain” refers to a sequence of amino acids which confers the ability of endosomally-trapped cargoes to gain access to the cytoplasmic compartment. Without being bound by theory, endosome leakage domains are short sequences (often derived from viral or bacterial peptides), which are believed to induce destabilization of the endosomal membrane and liberation of the endosome contents into the cytoplasm. As used herein, the expression “endosomolytic peptide” is intended to refer to this general class of peptides having endosomal membrane-destabilizing properties. Accordingly, in some embodiments, synthetic peptide or polypeptide-based shuttle agents of the present description may comprise an ELD which is an endosomolytic peptide. The activity of such peptides may be assessed for example using the calcein endosome escape assays described in Example 2 of WO/2016/161516.

In some embodiments, the ELD may be a peptide that disrupts membranes at acidic pH, such as pH-dependent membrane active peptide (PMAP) or a pH-dependent lytic peptide. For example, the peptides GALA and INF-7 are amphiphilic peptides that form alpha helixes when a drop in pH modifies the charge of the amino acids which they contain. More particularly, without being bound by theory, it is suggested that ELDs such as GALA induce endosomal leakage by forming pores and flip-flop of membrane lipids following conformational change due to a decrease in pH (Kakudo, Chaki et al., 2004, Li, Nicol et al., 2004). In contrast, it is suggested that ELDs such as INF-7 induce endosomal leakage by accumulating in and destabilizing the endosomal membrane (El-Sayed, Futaki et al., 2009). Accordingly, in the course of endosome maturation, the concomitant decline in pH causes a change in the conformation of the peptide and this destabilizes the endosome membrane leading to the liberation of the endosome contents. The same principle is thought to apply to the toxin A of Pseudomonas (Varkouhi, Scholte et al., 2011). Following a decline in pH, the conformation of the domain of translocation of the toxin changes, allowing its insertion into the endosome membrane where it forms pores (London 1992, O'Keefe 1992). This eventually favors endosome destabilization and translocation of the complex outside of the endosome. The above described ELDs are encompassed within the ELDs of the present description, as well as other mechanisms of endosome leakage whose mechanisms of action may be less well defined.

In some embodiments, the ELD may be an antimicrobial peptide (AMP) such as a linear cationic alpha-helical antimicrobial peptide (AMP). These peptides play a key role in the innate immune response due to their ability to strongly interact with bacterial membranes. Without being bound by theory, these peptides are thought to assume a disordered state in aqueous solution, but adopt an alpha-helical secondary structure in hydrophobic environments. The latter conformation thought to contribute to their typical concentration-dependent membrane-disrupting properties. When accumulated in endosomes at certain concentrations, some antimicrobial peptides may induce endosomal leakage.

In some embodiments, the ELD may be an antimicrobial peptide (AMP) such as Cecropin-A/Melittin hybrid (CM) peptide. Such peptides are thought to be among the smallest and most effective AMP-derived peptides with membrane-disrupting ability. Cecropins are a family of antimicrobial peptides with membrane-perturbing abilities against both Gram-positive and Gram-negative bacteria. Cecropin A (CA), the first identified antibacterial peptide, is composed of 37 amino acids with a linear structure. Melittin (M), a peptide of 26 amino acids, is a cell membrane lytic factor found in bee venom. Cecropin-melittin hybrid peptides have been shown to produce short efficient antibiotic peptides without cytotoxicity for eukaryotic cells (i.e., non-hemolytic), a desirable property in any antibacterial agent. These chimeric peptides were constructed from various combinations of the hydrophilic N-terminal domain of Cecropin A with the hydrophobic N-terminal domain of Melittin, and have been tested on bacterial model systems. Two 26-mers, CA(1-13)M(1-13) and CA(1-8) M(1-18) (Boman et al., 1989), have been shown to demonstrate a wider spectrum and improved potency of natural Cecropin A without the cytotoxic effects of melittin.

In an effort to produce shorter CM series peptides, the authors of Andreu et al., 1992 constructed hybrid peptides such as the 26-mer (CA(1-8)M(1-18)), and compared them with a 20-mer (CA(1-8)M(1-12)), a 18-mer (CA(1-8)M(1-10)) and six 15-mers ((CA(1-7)M(1-8), CA(1-7)M(2-9), CA(1-7)M(3-10), CA(1-7)M(4-11), CA(1-7)M(5-12), and CA(1-7)M(6-13)). The 20 and 18-mers maintained similar activity comparatively to CA(1-8)M(1-18). Among the six 15-mers, CA(1-7)M(1-8) showed low antibacterial activity, but the other five showed similar antibiotic potency compared to the 26-mer without hemolytic effect. Accordingly, in some embodiments, synthetic peptide or polypeptide-based shuttle agents of the present description may comprise an ELD which is or is from CM series peptide variants, such as those described above.

In some embodiments, the ELD may be the CM series peptide CM18 composed of residues 1-7 of Cecropin-A (KWKLFKKIGAVLKVLTTG) (SEQ ID NO.: 363) fused to residues 2-12 of Melittin (YGRKKRRQRRR) (SEQ ID NO: 364), [C(1-7)M(2-12)](SEQ ID NO: 365). When fused to the cell penetrating peptide TAT, CM18 was shown to independently cross the plasma membrane and destabilize the endosomal membrane, allowing some endosomally-trapped cargoes to be released to the cytosol (Salomone et al., 2012). However, the use of a CM18-TAT11 peptide fused to a fluorophore (atto-633) in some of the authors' experiments, raises uncertainty as to the contribution of the peptide versus the fluorophore, as the use of fluorophores themselves have been shown to contribute to endosomolysis—e.g., via photochemical disruption of the endosomal membrane (Erazo-Oliveras et al., 2014).

In some embodiments, the ELD may be CM18 having the amino acid sequence of SEQ ID NO: 1 of WO/2016/161516, or a variant thereof having at least 70%, 71%, 72%, 73%, 74%, 75%, 76%, 77%, 78%, 79%, 80%, 81%, 82%, 83%, 84%, 85%, 86%, 87%, 88%, 89%, 85%, 90%, 91%, 92%, 93%, 94%, or 95% identity to SEQ ID NO: 1 of WO/2016/161516 and having endosomolytic activity.

In some embodiments, the ELD may be a peptide derived from the N terminus of the HA2 subunit of influenza hemagglutinin (HA), which may also cause endosomal membrane destabilization when accumulated in the endosome.

In some embodiments, synthetic peptide or polypeptide-based shuttle agents of the present description may comprise an ELD which is or is from an ELD set forth in Table I, or a variant thereof having endosome escape activity and/or pH-dependent membrane disrupting activity.

TABLE I
Examples of endosome leakage domains

| | SEQ ID NO of | |
|---|---|---|
| Name | WO/2016/161516 | Reference(s) |
| CM18 | 1 | Salomone, Cardarelli et |
| | | al., 2012 |
| Diphtheria toxin T | 2 | Uherek, Fominaya et al., |
| domain (DT) | | 1998, Glover, Ng et al., |
| | | 2009 |
| GALA | 3 | Parente, Nir et al., 1990 |
| | | Li, Nicol et al., 2004 |
| PEA | 4 | Fominaya and Wels 1996 |
| INF-7 | 5 | El-Sayed, Futaki et al., |
| | | 2009 |
| LAH4 | 6 | Kichler, Mason et al., |
| | | 2006 |
| | | Kichler et al., 2003 |
| HGP | 7 | Kwon et al., 2010 |
| H5WYG | 8 | Midoux, Kichler et al., |
| | | 1998 |
| HA2 | 9 | Lorieau, Louis et al., 2010 |
| EB1 | 10 | Amand, Norden et al., |
| | | 2012 |
| VSVG | 11 | Schuster, Wu et al., 1999 |
| Pseudomonas toxin | 12 | Fominaya, Uherek et al., |
| | | 1998 |
| Melittin | 13 | Tan, Chen et al., 2012 |
| KALA | 14 | Wyman, Nicol et al., 1997 |
| JST-1 | 15 | Gottschalk, Sparrow et al., |
| | | 1996 |
| C(LLKK)₃C (SEQ | 63 | Luan et al., 2015 |
| ID NO: 366) | | |
| G(LLKK)₃G (SEQ | 64 | Luan et al., 2015 |
| ID NO: 367) | | |

In some embodiments, shuttle agents of the present description may comprise one or more ELD or type of ELD. More particularly, they can comprise at least 2, at least 3, at least 4, at least 5, or more ELDs. In some embodiments, the shuttle agents can comprise between 1 and 10 ELDs, between 1 and 9 ELDs, between 1 and 8 ELDs, between 1 and 7 ELDs, between 1 and 6 ELDs, between 1 and 5 ELDs, between 1 and 4 ELDs, between 1 and 3 ELDs, etc.

In some embodiments, the order or placement of the ELD relative to the other domains (CPD, histidine-rich domains) within the shuttle agents of the present description may be varied provided the shuttling ability of the shuttle agent is retained.

In some embodiments, the ELD may be a variant or fragment of any one those listed in Table I, and having endosomolytic activity. In some embodiments, the ELD may comprise or consist of the amino acid sequence of any one of SEQ ID NOs: 1-15, 63, or 64 of WO/2016/161516, or a sequence which is at least 70%, 71%, 72%, 73%, 74%, 75%, 76%, 77%, 78%, 79%, 80%, 81%, 82%, 83%, 84%, 85%, 86%, 87%, 88%, 89%, 85%, 90%, 91%, 92%, 93%, 94%, or 95% identical to any one of SEQ ID NOs: 1-15, 63, or 64 of WO/2016/161516, and having endosomolytic activity.

In some embodiments, shuttle agents of the present description do not comprise one or more of the amino acid sequences of any one of SEQ ID NOs: 1-15, 63, or 64 of WO/2016/161516.

### Cell Penetration Domains (CPDs)

In some aspects, the shuttle agents of the present description may comprise a cell penetration domain (CPD). As used herein, the expression “cell penetration domain” refers to a sequence of amino acids which confers the ability of a macromolecule (e.g., peptide or protein) containing the CPD to be transduced into a cell.

In some embodiments, the CPD may be (or may be from) a cell-penetrating peptide or the protein transduction domain of a cell-penetrating peptide. Cell-penetrating peptides can serve as carriers to successfully deliver a variety of cargoes intracellularly (e.g., polynucleotides, polypeptides, small molecule compounds or other macromolecules/compounds that are otherwise membrane-impermeable). Cell-penetrating peptides often include short peptides rich in basic amino acids that, once fused (or otherwise operably linked) to a macromolecule, mediate its internalization inside cells (Shaw, Catchpole et al., 2008). The first cell-penetrating peptide was identified by analyzing the cell penetration ability of the HIV-1 trans-activator of transcription (Tat) protein (Green and Loewenstein 1988, Vives, Brodin et al., 1997). This protein contains a short hydrophilic amino acid sequence, named “TAT”, which promotes its insertion within the plasma membrane and the formation of pores. Since this discovery, many other cell-penetrating peptides have been described. In this regard, in some embodiments, the CPD can be a cell-penetrating peptide as listed in Table II, or a variant thereof having cell-penetrating activity.

TABLE II
Examples of cell-penetrating peptides

| | SEQ ID NO of | |
|---|---|---|
| Name | WO/2016/161516 | Reference(s) |
| SP | 16 | Mahlum, Mandal et al., 2007 |
| TAT | 17 | Green and Loewenstein 1988, |
| | | Fawell, Seery et al., 1994, |
| | | Vives, Brodin et al., 1997 |
| Penetratin | 18 | Perez, Joliot et al., 1992 |
| (Antennapedia) | | |
| pVEC | 19 | Elmquist, Lindgren et al., 2001 |
| M918 | 20 | El-Andaloussi, Johansson et |
| | | al., 2007 |
| Pep-1 | 21 | Morris, Depollier et al., 2001 |
| Pep-2 | 22 | Morris, Chaloin et al., 2004 |
| Xentry | 23 | Montrose, Yang et al., 2013 |
| Arginine stretch | 24 | Zhou, Wu et al., 2009 |
| Transportan | 25 | Hallbrink, Floren et al., 2001 |
| SynB1 | 26 | Drin, Cottin et al., 2003 |
| SynB3 | 27 | Drin, Cottin et al., 2003 |
| PTD4 | 65 | Ho et al, 2001 |

Without being bound by theory, cell-penetrating peptides are thought to interact with the cell plasma membrane before crossing by pinocytosis or endocytosis. In the case of the TAT peptide, its hydrophilic nature and charge are thought to promote its insertion within the plasma membrane and the formation of a pore (Herce and Garcia 2007). Alpha helix motifs within hydrophobic peptides (such as SP) are also thought to form pores within plasma membranes (Veach, Liu et al., 2004).

In some embodiments, shuttle agents of the present description may comprise one or more CPD or type of CPD. More particularly, they may comprise at least 2, at least 3, at least 4, or at least 5 or more CPDs. In some embodiments, the shuttle agents can comprise between 1 and 10 CPDs, between 1 and 6 CPDs, between 1 and 5 CPDs, between 1 and 4 CPDs, between 1 and 3 CPDs, etc.

In some embodiments, the CPD may be TAT having the amino acid sequence of SEQ ID NO: 17 of WO/2016/161516, or a variant thereof having at least 70%, 71%, 72%, 73%, 74%, 75%, 76%, 77%, 78%, 79%, 80%, 81% 82%, 83%, 84%, 85%, 86%, 87%, 88%, 89%, 90%, 91%, 92%, 93%, 94%, or 95% identity to SEQ ID NO: 17 of WO/2016/161516 and having cell penetrating activity; or Penetratin having the amino acid sequence of SEQ ID NO: 18 of WO/2016/161516, or a variant thereof having at least 70%, 71%, 72%, 73%, 74%, 75%, 76%, 77%, 78%, 79%, 80%, 81% 82%, 83%, 84%, 85%, 86%, 87%, 88%, 89%, 85%, 86%, 87%, 88%, 89%, 90%, 91%, 92%, 93%, 94%, or 95% identity to SEQ ID NO: 18 of WO/2016/161516 and having cell penetrating activity.

In some embodiments, the CPD may be PTD4 having the amino acid sequence of SEQ ID NO: 65 of WO/2016/161516, or a variant thereof having at least 70%, 71%, 72%, 73%, 74%, 75%, 76%, 77%, 78%, 79%, 80%, 81% 82%, 83%, 84%, 85%, 86%, 87%, 88%, 89%, 90%, 91%, 92%, 93%, 94%, or 95% identity to SEQ ID NO: 65 of WO/2016/161516.

In some embodiments, the order or placement of the CPD relative to the other domains (ELD, histidine-rich domains) within the shuttle agents of the present description may be varied provided the transduction ability of the shuttle agent is retained.

In some embodiments, the CPD may be a variant or fragment of any one those listed in Table II, and having cell penetrating activity. In some embodiments, the CPD may comprise or consist of the amino acid sequence of any one of SEQ ID NOs: 16-27 or 65 of WO/2016/161516, or a sequence which is at least 70%, 71%, 72%, 73%, 74%, 75%, 76%, 77%, 78%, 79%, 80%, 81%, 82%, 83%, 84%, 85%, 86%, 87%, 88%, 89%, 85%, 90%, 91%, 92%, 93%, 94%, or 95% identical to any one of SEQ ID NOs: 16-27 or 65 of WO/2016/161516, and having cell penetrating activity.

In some embodiments, shuttle agents of the present description do not comprise any one of the amino acid sequences of SEQ ID NOs: 16-27 or 65 of WO/2016/161516.

### Methods, Kits, Uses, Compositions, and Cells

In some embodiments, the present description relates to methods for delivering a proteinaceous and/or non-proteinaceous cargo from an extracellular space to the cytosol and/or nucleus of a target eukaryotic cell. The methods comprise contacting the target eukaryotic cell with the cargo in the presence of a shuttle agent at a concentration sufficient to increase the transduction efficiency of said cargo, as compared to in the absence of said shuttle agent. In some embodiments, contacting the target eukaryotic cell with the cargo in the presence of the shuttle agent results in an increase in the transduction efficiency of said non-proteinaceous cargo by at least 10-fold, 20-fold, 30-fold, 40-fold, 50-fold, or 100-fold, as compared to in the absence of said shuttle agent.

In some embodiments, the present description relates to a method for increasing the transduction efficiency of a proteinaceous and/or non-proteinaceous cargo to the cytosol and/or nucleus of target eukaryotic cells. As used herein, the expression “increasing transduction efficiency” refers to the ability of a shuttle agent of the present description to improve the percentage or proportion of a population of target cells into which a cargo of interest (e.g., non-proteinaceous cargo) is delivered intracellularly. Immunofluorescence microscopy, flow cytometry, and other suitable methods may be used to assess cargo transduction efficiency. In some embodiments, a shuttle agent of the present description may enable a transduction efficiency of at least 25%, 30%, 35%, 40%, 45%, 50%, 55%, 60%, 65%, 70%, 75%, 80%, or 85%, for example as measured by immunofluorescence microscopy, flow cytometry, FACS, and other suitable methods. In some embodiments, a shuttle agent of the present description may enable one of the aforementioned transduction efficiencies together wish a cell viability of at least 25%, 30%, 35%, 40%, 45%, 50%, 55%, 60%, 65%, 70%, 75%, 80%, 85%, 90%, or 95%, for example as measured by the assay described in Example 3.3a of WO/2018/068135, or by another suitable assay known in the art.

In addition to increasing target cell transduction efficiency, shuttle agents of the present description may facilitate the delivery of a cargo of interest (e.g., a proteinaceous and/or non-proteinaceous cargo) to the cytosol and/or nucleus of target cells. In this regard, efficiently delivering an extracellular cargo to the cytosol and/or nucleus of a target cell using peptides can be challenging, as the cargo often becomes trapped in intracellular endosomes after crossing the plasma membrane, which may limit its intracellular availability and may result in its eventual metabolic degradation. For example, use of the protein transduction domain from the HIV-1 Tat protein has been reported to result in massive sequestration of the cargo into intracellular vesicles. In some aspects, shuttle agents of the present description may facilitate the ability of endosomally-trapped cargo to escape from the endosome and gain access to the cytoplasmic compartment. In this regard, the expression “to the cytosol” for example in the phrase “increasing the transduction efficiency of a non-proteinaceous cargo to the cytosol,” is intended to refer to the ability of shuttle agents of the present description to allow an intracellularly delivered cargo of interest to escape endosomal entrapment and gain access to the cytoplasmic and/or nuclear compartment. After a cargo of interest has gained access to the cytosol, it may be free to bind to its intracellular target (e.g., nucleus, nucleolus, mitochondria, peroxisome). In some embodiments, the expression “to the cytosol” is thus intended to encompass not only cytosolic delivery, but also delivery to other subcellular compartments that first require the cargo to gain access to the cytoplasmic compartment.

In some embodiments, the methods of the present description are in vitro methods (e.g., such as for therapeutic and/or diagnostic purpose). In other embodiments, the methods of the present description are in vivo methods (e.g., such as for therapeutic and/or diagnostic purpose). In some embodiments, the methods of the present description comprise topical, enteral/gastrointestinal (e.g., oral), or parenteral administration of the non-proteinaceous cargo and the synthetic peptide shuttle agent. In some embodiments, described herein are compositions formulated for topical, enteral/gastrointestinal (e.g., oral), or parenteral administration of the non-proteinaceous cargo and the synthetic peptide shuttle agent.

In some embodiments, the methods of the present description may comprise contacting the target eukaryotic cell with the shuttle agent, or composition as defined herein, and the proteinaceous and/or non-proteinaceous cargo. In some embodiments, the shuttle agent, or composition may be pre-incubated with the cargo to form a mixture, prior to exposing the target eukaryotic cell to that mixture. In some embodiments, the type of shuttle agent may be selected based on the identity and/or physicochemical properties of the cargo to be delivered intracellularly. In other embodiments, the type of shuttle agent may be selected to take into account the identity and/or physicochemical properties of the cargo to be delivered intracellularly, the type of cell, the type of tissue, etc.

In some embodiments, the method may comprise multiple treatments of the target cells with the shuttle agent, or composition (e.g., 1, 2, 3, 4 or more times per day, and/or on a pre-determined schedule). In such cases, lower concentrations of the shuttle agent, or composition may be advisable (e.g., for reduced toxicity). In some embodiments, the cells may be suspension cells or adherent cells. In some embodiments, the person of skill in the art will be able to adapt the teachings of the present description using different combinations of shuttles, domains, uses and methods to suit particular needs of delivering a proteinaceous and/or non-proteinaceous cargo to particular cells with a desired viability.

In some embodiments, the methods of the present description may apply to methods of delivering a proteinaceous and/or non-proteinaceous cargo intracellularly to a cell in vivo. Such methods may be accomplished by parenteral administration or direct injection into a tissue, organ, or system.

In some aspects, the synthetic peptide shuttle agents of the present description may be for use in an in vitro or in vivo method for increasing the transduction efficiency of a proteinaceous and/or non-proteinaceous cargo (e.g., a therapeutically or biologically active proteinaceous and/or non-proteinaceous cargo) into target eukaryotic cells, wherein the synthetic peptide shuttle agent or synthetic peptide shuttle agent variant is used or is formulated for use at a concentration sufficient to increase the transduction efficiency and cytosolic and/or nuclear delivery of the cargo into the target eukaryotic cells, as compared to in the absence of the synthetic peptide shuttle agent or synthetic peptide shuttle agent variant.

In some embodiments, synthetic peptide shuttle agents of the present description maybe for use in therapy, wherein the synthetic peptide shuttle agent or synthetic peptide shuttle agent variant transduces a therapeutically or biologically active proteinaceous and/or non-proteinaceous cargo to the cytosol and/or nucleus of target eukaryotic cells, wherein the synthetic peptide shuttle agent or synthetic peptide shuttle agent variant is used (or is formulated for use) at a concentration sufficient to increase the transduction efficiency of the cargo into the target eukaryotic cells, as compared to in the absence of the synthetic peptide shuttle agent.

In some aspects, described herein is a composition for use in transducing a proteinaceous and/or non-proteinaceous cargo into target eukaryotic cells, the composition comprising a synthetic peptide shuttle agent formulated with a pharmaceutically suitable excipient, wherein the concentration of the synthetic peptide shuttle agent in the composition is sufficient to increase the transduction efficiency and cytosolic and/or nuclear delivery of the cargo into said target eukaryotic cells upon administration, as compared to in the absence of said synthetic peptide shuttle agent. In some embodiments, the composition further comprises the cargo. In some embodiments, the composition may be mixed with the cargo prior to administration or therapeutic use.

In some aspects, described herein is a composition for use in therapy, the composition comprising a synthetic peptide shuttle agent formulated with a proteinaceous and/or non-proteinaceous cargo to be transduced into target eukaryotic cells by the synthetic peptide shuttle agent, wherein the concentration of the synthetic peptide shuttle agent in the composition is sufficient to increase the transduction efficiency and cytosolic and/or nuclear delivery of the cargo into said target eukaryotic cells upon administration, as compared to in the absence of said synthetic peptide shuttle agent.

In some embodiments, the shuttle agent, or composition, and the proteinaceous and/or non-proteinaceous cargo may be exposed to the target cell in the presence or absence of serum. In some embodiments, the method may be suitable for clinical or therapeutic use.

In some embodiments, the present description relates to a kit for delivering a proteinaceous and/or non-proteinaceous cargo from an extracellular space to the cytosol and/or nucleus of a target eukaryotic cell. In some embodiments, the present description relates to a kit for increasing the transduction efficiency of a proteinaceous and/or non-proteinaceous cargo to the cytosol of a target eukaryotic cell. The kit may comprise the shuttle agent, or composition as defined herein, and a suitable container.

In some embodiments, the target eukaryotic cells may be an animal cell, a mammalian cell, or a human cell. In some embodiments, the target eukaryotic cells may be stem cells (e.g., embryonic stem cells, pluripotent stem cells, induced pluripotent stem cells, neural stem cells, mesenchymal stem cells, hematopoietic stem cells, peripheral blood stem cells), primary cells (e.g., myoblast, fibroblast), immune cells (e.g., NK cell, T cell, dendritic cell, antigen presenting cell), epithelial cells, skin cells, gastrointestinal cells, mucosal cells, or pulmonary cells. In some embodiments, target cells comprise those having the cellular machinery for endocytosis (i.e., to produce endosomes).

In some embodiments, the present description relates to an isolated cell comprising a synthetic peptide shuttle agent as defined herein. In some embodiments, the cell may be a protein-induced pluripotent stem cell. It will be understood that cells that are often resistant or not amenable to DNA transfection may be interesting candidates for the synthetic peptide shuttle agents of the present description.

In some embodiments, the present description relates to a method for producing a synthetic peptide shuttle agent that delivers a proteinaceous and/or non-proteinaceous cargo from an extracellular space to the cytosol and/or nucleus of a target eukaryotic cell, the method comprising synthesizing a peptide which is: (1) a peptide at least 17, 18, 19, or 20 amino acids in length comprising (2) an amphipathic alpha-helical motif having (3) a positively-charged hydrophilic outer face, and a hydrophobic outer face, wherein at least five of the parameters (4) to (15) defined herein are respected.

In some embodiments, the present description relates to a method for identifying or selecting a shuttle agent that delivers a proteinaceous and/or non-proteinaceous cargo from an extracellular space to the cytosol and/or nucleus of a target eukaryotic cell, the method comprising: (a) synthesizing a peptide which is the peptide as defined herein; (b) contacting the target eukaryotic cell with the cargo in the presence of said peptide; (c) measuring the transduction efficiency of the cargo in the target eukaryotic cell; and (d) identifying or selecting the peptide as being a shuttle agent that transduces the cargo, when an increase in transduction activity (e.g., transduction efficiency) of said cargo in the target eukaryotic cell is observed.

In some aspects, the present description relates to a composition for use in transducing a proteinaceous and/or non-proteinaceous cargo into target eukaryotic cells, the composition comprising a synthetic peptide shuttle agent formulated with a pharmaceutically suitable excipient, wherein the concentration of the synthetic peptide shuttle agent in the composition is sufficient to increase the transduction efficiency and cytosolic delivery of the cargo into said target eukaryotic cells upon administration, as compared to in the absence of said synthetic peptide shuttle agent. In some embodiments, the composition further comprises the cargo.

In some embodiments, the present description relates to oral formulations comprising the shuttle agents described herein and a cargo as described herein, for example an enterically-coated oral dosage form.

In some embodiments, applications of the shuttle agents described herein in food, farming, and/or agricultural industries may be envisaged. In some embodiments, the shuttle agents described herein may be formulated as a feed additive to aid in weight gain and/or the absorption of nutrients. In some embodiments, the shuttle agents described herein may be formulated as a feed additive to aid in weight gain and/or the absorption of nutrients.

In some aspects, described herein is a process for producing a candidate synthetic peptide shuttle agent expected to have transduction activity for a proteinaceous and/or non-proteinaceous cargo of interest in target eukaryotic cells, the method comprising synthesizing a peptide which is: (1) a peptide at least 17, 18, 19, or 20 amino acids in length comprising (2) an amphipathic alpha-helical motif having (3) a positively-charged hydrophilic outer face, and a hydrophobic outer face, wherein at least five of parameters (4) to (15) as defined herein are respected, and wherein the shuttle agent increases the transduction efficiency of propidium iodide or other membrane-impermeable fluorescent DNA intercalating agent by at least 3, 3.5, 4, 4.5, 5, 5.5, 6, 6.5, 7, 7.5, 8, 8.5, 9, 9.5, or 10-fold over a corresponding negative control lacking said shuttle agent, and/or enables a transduction efficiency of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of propidium iodide or other membrane-impermeable fluorescent DNA intercalating agent, in a eukaryotic cell line model (e.g., HeLa) suitable for assessing cargo transduction in said target eukaryotic cells.

In some aspects, described herein is an in vitro or in vivo method for identifying or selecting a synthetic peptide shuttle agent expected to have transduction activity for proteinaceous and/or non-proteinaceous cargoes in target eukaryotic cells, the method comprising: providing model eukaryotic cells or a model organism suitable for assessing cargo transduction in the target eukaryotic cells; providing a candidate synthetic peptide shuttle agent (e.g., any shuttle agent as defined herein); and measuring the transduction activity (e.g., cargo transduction efficiency, such as by flow cytometry) of the candidate synthetic peptide shuttle agent to transduce propidium iodide or other membrane-impermeable fluorescent DNA intercalating agent into the eukaryotic cell line model, wherein the candidate shuttle agent is expected to have transduction activity for both proteinaceous and non-proteinaceous cargoes in the target eukaryotic cells when the transduction activity (e.g., transduction efficiency) of propidium iodide or other membrane-impermeable fluorescent DNA intercalating agent is increased by at least 3, 3.5, 4, 4.5, 5, 5.5, 6, 6.5, 7, 7.5, 8, 8.5, 9, 9.5, or 10-fold over a corresponding negative control lacking the candidate synthetic peptide shuttle agent, and/or a transduction efficiency of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of the propidium iodide or other membrane-impermeable fluorescent DNA intercalating agent occurs, in the model eukaryotic cells or model organism.

### Items I

In some aspects, described here are one or more of the following items:
1. A method for non-proteinaceous cargo transduction, the method comprising contacting target eukaryotic cells with a non-proteinaceous cargo and a concentration of a synthetic peptide shuttle agent sufficient to increase the transduction efficiency of said non-proteinaceous cargo, as compared to in the absence of said synthetic peptide shuttle agent.
2. The method of item 1, wherein the non-proteinaceous cargo: (a) is an organic compound; (b) has a molecular weight of less than 10 000, 9000, 8000, 7000, 6000, 5000, 4000, 3000, 2000, or 1000 Da, or between 50 to 5000, 50 to 4000, 50 to 3000, 50 to 2000, or 50 to 1000 Da; (c) is a small molecule, such as a small molecule drug that binds to an intracellular biological or therapeutic target; (d) is not a biopolymer, such as a polynucleotide or a polysaccharide; (e) is not covalently linked to the synthetic peptide shuttle agent at the moment of transduction; or (f) any combination of (a) to (e).
3. The method of item 1 or 2, wherein non-proteinaceous cargo is a drug for treating cancer (e.g., skin cancer, basal cell carcinoma, nevoid basal cell carcinoma syndrome), inflammation or an inflammation-related disease (e.g., psoriasis, atopic dermatitis, ulcerative colitis, urticaria, dry eye disease, dry or wet age-related macular degeneration, digital ulcers, actinic keratosis, idiopathic pulmonary fibrosis), pain (e.g., chronic or acute), or a disease affecting the lungs (e.g., cystic fibrosis, asthma, chronic obstructive pulmonary disease (COPD), or idiopathic pulmonary fibrosis).
4. The method of any one of items 1 to 3, wherein non-proteinaceous cargo is or comprises a HedgeHog inhibitor (e.g., itraconazole, posaconazole, arsenic trioxide (ATO), Gant61, PF-4708671, HPI-1, HPI-4), a pain inhibitor such as a voltage-gated sodium (Nav) channel inhibitor (e.g., QX-314), and/or an inhibitor of inflammation (e.g., an inhibitor of inflammatory cytokine production, or an NF-kappa B pathway inhibitor).
5. The method of any one of items 1 to 4, wherein the shuttle agent is: (1) a peptide at least 20 amino acids in length comprising (2) an amphipathic alpha-helical motif having (3) a positively-charged hydrophilic outer face, and a hydrophobic outer face, wherein at least five of the following parameters (4) to (15) are respected: (4) the hydrophobic outer face comprises a highly hydrophobic core consisting of spatially adjacent L, I, F, V, W, and/or M amino acids representing 12 to 50% of the amino acids of the peptide, based on an open cylindrical representation of the alpha-helix having 3.6 residues per turn; (5) the peptide has a hydrophobic moment (μ) of 3.5 to 11; (6) the peptide has a predicted net charge of at least +4 at physiological pH; (7) the peptide has an isoelectric point (pI) of 8 to 13; (8) the peptide is composed of 35% to 65% of any combination of the amino acids: A, C, G, I, L, M, F, P, W, Y, and V; (9) the peptide is composed of 0% to 30% of any combination of the amino acids: N, Q, S, and T; (10) the peptide is composed of 35% to 85% of any combination of the amino acids: A, L, K, or R; (11) the peptide is composed of 15% to 45% of any combination of the amino acids: A and L, provided there being at least 5% of L in the peptide; (12) the peptide is composed of 20% to 45% of any combination of the amino acids: K and R; (13) the peptide is composed of 0% to 10% of any combination of the amino acids: D and E; (14) the difference between the percentage of A and L residues in the peptide (% A+L), and the percentage of K and R residues in the peptide (K+R), is less than or equal to 10%; and (15) the peptide is composed of 10% to 45% of any combination of the amino acids: Q, Y, W, P, I, S, G, V, F, E, D, C, M, N, T and H.
6. The method of item 5, wherein: (a) the shuttle agent respects at least six, at least seven, at least eight, at least nine, at least ten, at least eleven, or respects all of parameters (4) to (15); (b) the shuttle agent is a peptide having a minimum length of 20, 21, 22, 23, 24, 25, 26, 27, 28, 29, or 30 amino acids, and a maximum length of 40, 41, 42, 43, 44, 45, 46, 47, 48, 49, 50, 51, 52, 53, 54, 55, 60, 65, 70, 80, 90, 100, 110, 120, 130, 140, or 150 amino acids; (c) said amphipathic alpha-helical motif has a hydrophobic moment (μ) between a lower limit of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, 10.4, 10.5, 10.6, 10.7, 10.8, 10.9, or 11.0; (d) said amphipathic alpha-helical motif comprises a positively-charged hydrophilic outer face comprising: (i) at least two, three, or four adjacent positively-charged K and/or R residues upon helical wheel projection; and/or (ii) a segment of six adjacent residues comprising three to five K and/or R residues upon helical wheel projection, based on an alpha helix having angle of rotation between consecutive amino acids of 100 degrees and/or an alpha-helix having 3.6 residues per turn; (e) said amphipathic alpha-helical motif comprises a hydrophobic outer face comprising: (i) at least two adjacent L residues upon helical wheel projection; and/or (ii) a segment often adjacent residues comprising at least five hydrophobic residues selected from: L, I, F, V, W, and M, upon helical wheel projection, based on an alpha helix having angle of rotation between consecutive amino acids of 100 degrees and/or an alpha-helix having 3.6 residues per turn; (f) said hydrophobic outer face comprises a highly hydrophobic core consisting of spatially adjacent L, I, F, V, W, and/or M amino acids representing from 12.5%, 13%, 13.5%, 14%, 14.5%, 15%, 15.5%, 16%, 16.5%, 17%, 17.5%, 18%, 18.5%, 19%, 19.5%, or 20%, to 25%, 30%, 35%, 40%, or 45% of the amino acids of the shuttle agent; (g) the shuttle agent has a hydrophobic moment (μ) between a lower limit of 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, 10.4, or 10.5; (h) the shuttle agent has a predicted net charge of between +4, +5, +6, +7, +8, +9, to +10, +11, +12, +13, +14, or +15; (i) the shuttle agent has a predicted pI of 10 to 13; or (j) any combination of (a) to (i).
7. The method of any one of items 1 to 6, wherein said shuttle agent respects at least one, at least two, at least three, at least four, at least five, at least six, or all of the following parameters: (8) the shuttle agent is composed of 36% to 64%, 37% to 63%, 38% to 62%, 39% to 61%, or 40% to 60% of any combination of the amino acids: A, C, G, I, L, M, F, P, W, Y, and V; (9) the shuttle agent is composed of 1% to 29%, 2% to 28%, 3% to 27%, 4% to 26%, 5% to 25%, 6% to 24%, 7% to 23%, 8% to 22%, 9% to 21%, or 10% to 20% of any combination of the amino acids: N, Q, S, and T; (10) the shuttle agent is composed of 36% to 80%, 37% to 75%, 38% to 70%, 39% to 65%, or 40% to 60% of any combination of the amino acids: A, L, K, or R; (11) the shuttle agent is composed of 15% to 40%, 20% to 40%, 20 to 35%, or 20 to 30% of any combination of the amino acids: A and L; (12) the shuttle agent is composed of 20% to 40%, 20 to 35%, or 20 to 30% of any combination of the amino acids: K and R; (13) the shuttle agent is composed of 5 to 10% of any combination of the amino acids: D and E; (14) the difference between the percentage of A and L residues in the shuttle agent (% A+L), and the percentage of K and R residues in the shuttle agent (K+R), is less than or equal to 9%, 8%, 7%, 6%, or 5%; and (15) the shuttle agent is composed of 15 to 40%, 20% to 35%, or 20% to 30% of any combination of the amino acids: Q, Y, W, P, I, S, G, V, F, E, D, C, M, N, T, and H.
8. The method of any one of items 1 to 7, wherein said shuttle agent comprises a histidine-rich domain, optionally wherein the histidine-rich domain is: (i) positioned towards the N terminus and/or towards the C terminus of the shuttle agent; (ii) is a stretch of at least 3, at least 4, at least 5, or at least 6 amino acids comprising at least 50%, at least 55%, at least 60%, at least 65%, at least 70%, at least 75%, at least 80%, at least 85%, or at least 90% histidine residues; and/or comprises at least 2, at least 3, at least 4, at least 5, at least 6, at least 7, at least 8, or at least 9 consecutive histidine residues (SEQ ID NO: 368); or (iii) both (i) and (ii).
9. The method of any one of items 1 to 8, wherein said shuttle agent comprises a flexible linker domain rich in serine and/or glycine residues.
10. The method of any one of items 1 to 9, wherein said shuttle agent comprises or consists of the amino acid sequence of (a) [X1]-[X2]-[linker]-[X3]-[X4](Formula 1); (b) [X1]-[X2]-[linker]-[X4]-[X3](Formula 2); (c) [X2]-[X1]-[linker]-[X3]-[X4](Formula 3); (d) [X2]-[X1]-[linker]-[X4]-[X3](Formula 4); (e) [X3]-[X4]-[linker]-[X1]-[X2](Formula 5); (f) [X3]-[X4]-[linker]-[X2]-[X1](Formula 6); (g) [X4]-[X3]-[linker]-[X1]-[X2](Formula 7); or (h) [X4]-[X3]-[linker]-[X2]-[X1](Formula 8), wherein: [X1] is selected from: 2[Φ]-1[+]-2[Φ]-1[ζ]-1[+]-; 2[Φ]-1[+]-2[Φ]-2[+]-; 1[+]-1[ζ]-1[+]-2[ζ]-1[ζ]-1[+]-; and 1[+]-1[Φ]-1[+]-2[Φ]-2[+]-; [X2] is selected from: -2[Φ]-1[+]-2[Φ]-2[ζ]-; -2[Φ]-1[+]-2[Φ]-2[+]-; -2[Φ]-1[+]-2[Φ]- 1[+]-1[ζ]-; -2[Φ]-1[+]-2[Φ]-1[ζ]-1[+]-; -2[ζ]-2[+]-1[Φ]-2[+]-; -2[Φ]-2[+]-1[Φ]-2[ζ]-; -2[Φ]-2[+]-1[Φ]-1[+]-1[ζ]-; and -2[Φ]-2[+]-1[Φ]-1[ζ]-1[+]-; [X3] is selected from: -4[+]-A-; -3[+]-G-A-; -3[+]-A-A-; -2[+]-1[Φ]-1[+]-A-; -2[+]-1[Φ]-G-A-; -2[+]-1[Φ]-A-A-; or -2[+]-A-1[+]-A; -2[+]-A-G-A; -2[+]-A-A-A-; -1[Φ]-3[+]-A-; -1[Φ]-2[+]-G-A-; -1[Φ]-2[+]-A-A-; -1[Φ]-1[+]-1[Φ]-1[+]-A; -1[Φ]-1[+]-1[Φ]-G-A; -1[Φ]-1[+]-1[Φ]-A-A; -1[Φ]-1[+]-A-1[+]-A; -1[Φ]-1[+]-A-G-A; -1[Φ]-1[+]-A-A-A; -A-1[+]-A-1[+]-A; -A-1[+]-A-G-A (SEQ ID NO: 345); and -A-1[+]-A-A-A (SEQ ID NO: 346); [X4] is selected from: -1[ζ]-2A-1[+]-A; -1[ζ]-2A-2[+]; -1[+]-2A-1[+]-A; -1[ζ]-2A-1[+]-1[ζ]-A-1[+]; -1[ζ]-A-1[ζ]-A-1[+]; -2[+]-A-2[+]; -2[+]-A-1[+]-A; -2[+]-A-1[+]-1[ζ]-A-1[+]; -2[+]-1[ζ]-A-1[+]; -1[+]-1[ζ]-A-1[+]-A; -1[+]-1[ζ]-A-2[+]; -1[+]-1[ζ]-A-1[+]-1[ζ]- A-1[+]; -1[+]-2[ζ]-A-1[+]; -1[+]-2[ζ]-2[+]; -1[+]-2[ζ]-1[+]-A; -1[+]-2[ζ]-1[+]-1[ζ]-A-1[+]; -1[+]-2[ζ]-1[ζ]-A-1[+]; -3[ζ]-2[+]; -3[ζ]-1[+]-A; -3[ζ]-1-[+]-1[ζ]-2A-1[+]-A; -1[ζ]-2A-2[+]; -1[ζ]-2A-1[+]-1[ζ]-A-1[+]; -2[+]-A-1[+]-A; -2[+]-1[ζ]-1[+]-A; -1[+]-1[ζ]-A-1[+]-A; -1[+]-2A-1[+]-1[ζ]-A-1[+]; and -1[ζ]-A-1[ζ]-A-1[+]; and [linker] is selected from: -Gn- (SEQ ID NO: 347); -Sn- (SEQ ID NO: 348); -(GnSn)n- (SEQ ID NO: 349); -(GnSn)nGn-(SEQ ID NO: 350); -(GnSn)nSn- (SEQ ID NO: 351); -(GnSn)nGn(GnSn)n- (SEQ ID NO: 352);
and -(GnSn)nSn(GnSn)n- (SEQ ID NO: 353); wherein: [Φ] is an amino acid which is: Leu, Phe, Trp, Ile, Met, Tyr, or Val, preferably Leu, Phe, Trp, or Ile; [+] is an amino acid which is: Lys or Arg; [ζ] is an amino acid which is: Gln, Asn, Thr, or Ser; A is the amino acid Ala; G is the amino acid Gly; S is the amino acid Ser; and n is an integer from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 1 to 4, or 1 to 3.
11. The method of any one of items 1 to 10, wherein the shuttle agent comprises or consists of a peptide which is at least 50%, 55%, 60%, 65%, 70%, 75%, 80%, 85%, 90%, or 95% identical to the amino acid sequence of any one of SEQ ID NOs: 19-50.
12. The method of any one of items 1 to 11, wherein the shuttle agent comprises an endosome leakage domain (ELD), and/or a cell penetrating domain (CPD).
13. The method of any one of items 1 to 12, wherein: (i) said ELD is or is from: an endosomolytic peptide; an antimicrobial peptide (AMP); a linear cationic alpha-helical antimicrobial peptide; a Cecropin-A/Melittin hybrid (CM) peptide; pH-dependent membrane active peptide (PAMP); a peptide amphiphile; a peptide derived from the N terminus of the HA2 subunit of influenza hemagglutinin (HA); CM18; Diphtheria toxin T domain (DT); GALA; PEA; INF-7; LAH4; HGP; H5WYG; HA2; EBi; VSVG; Pseudomonas toxin; melittin; KALA; JST-1; C(LLKK)₃C (SEQ ID NO: 366); G(LLKK)₃G (SEQ ID NO: 367); or any combination thereof, (ii) said CPD is or is from: a cell-penetrating peptide or the protein transduction domain from a cell-penetrating peptide; TAT; PTD4; Penetratin; pVEC; M918; Pep-1; Pep-2; Xentry; arginine stretch; transportan; SynB1; SynB3; or any combination thereof; or (iii) both (i) and (ii).
14. The method of any one of items 1 to 13, wherein the shuttle agent is a cyclic peptide and/or comprises one or more D-amino acids.
15. The method of any one of items 1 to 14, which is an in vitro method, such as for therapeutic and/or diagnostic purpose.
16. The method of any one of items 1 to 14, which is an in vivo method, such as for therapeutic and/or diagnostic purpose.
17. The method of item 16 comprising topical, enteral/gastrointestinal (e.g., oral), or parenteral administration of the non-proteinaceous cargo and the synthetic peptide shuttle agent.
18. A composition for use in transducing a non-proteinaceous cargo into target eukaryotic cells, the composition comprising a synthetic peptide shuttle agent formulated with a pharmaceutically suitable excipient, wherein the concentration of the synthetic peptide shuttle agent in the composition is sufficient to increase the transduction efficiency and cytosolic delivery of the non-proteinaceous cargo into said target eukaryotic cells upon administration, as compared to in the absence of said synthetic peptide shuttle agent.
19. The composition of item 17, further comprising the non-proteinaceous cargo.
20. The composition of item 18 or 19, wherein: (a) the synthetic peptide shuttle agent is as defined in any one of items 1 or 5 to 14; (b) the non-proteinaceous cargo is as defined in any one of items 2 to 4; (c) the composition is for use in the in vitro or in vivo method as defined in any one of items 15 to 17; or (d) any combination of (a) to (c).
21. A kit for use in the method of any one of items 1 to 17, the kit comprising the synthetic peptide shuttle agent is as defined in any one of items 1 or 5 to 14, and the non-proteinaceous cargo is as defined in any one of items 2 to 4.
22. The method of any one of items 1 to 17, the composition of any one of items 18 to 20, or the kit of item 21, wherein the target eukaryotic cells are animal cells, mammalian cells, human cells, stem cells, primary cells, immune cells, T cells, NK cells, dendritic cells, epithelial cells, skin cells, or gastrointestinal cells.
23. A synthetic peptide shuttle agent having transduction activity for both proteinaceous and non-proteinaceous cargoes, the shuttle agent comprising an amino acid sequence at least 50%, 55%, 60%, 65%, 70%, 75%, 80%, 85%, 90%, or 95% identical to any one of SEQ ID NOs: 19-50.
24. The synthetic peptide shuttle agent of item 23, which is the shuttle agent as defined in any one of items 5 to 13.

### Items II

In some aspects, described here are one or more of the following items:
1. A method for non-proteinaceous cargo transduction, the method comprising contacting target eukaryotic cells with a non-proteinaceous cargo and a concentration of a synthetic peptide shuttle agent sufficient to increase the transduction efficiency of said non-proteinaceous cargo, as compared to in the absence of said synthetic peptide shuttle agent.
2. The method of item 1, wherein the non-proteinaceous cargo: (a) is an organic compound; (b) has a molecular weight of less than 10 000, 9000, 8000, 7000, 6000, 5000, 4000, 3000, 2000, or 1000 Da, or between 50 to 5000, 50 to 4000, 50 to 3000, 50 to 2000, or 50 to 1000 Da; (c) is a small molecule, such as a small molecule drug that binds to an intracellular biological or therapeutic target; (d) is not a biopolymer, such as a polynucleotide or a polysaccharide; (e) is not covalently linked to the synthetic peptide shuttle agent at the moment of transduction; or (f) any combination of (a) to (e).
3. The method of item 1 or 2, wherein non-proteinaceous cargo is a drug for treating cancer (e.g., skin cancer, basal cell carcinoma, nevoid basal cell carcinoma syndrome), inflammation or an inflammation-related disease (e.g., psoriasis, atopic dermatitis, ulcerative colitis, urticaria, dry eye disease, dry or wet age-related macular degeneration, digital ulcers, actinic keratosis, idiopathic pulmonary fibrosis), pain (e.g., chronic or acute), or a disease affecting the lungs (e.g., cystic fibrosis, asthma, chronic obstructive pulmonary disease (COPD), or idiopathic pulmonary fibrosis).
4. The method of any one of items 1 to 3, wherein non-proteinaceous cargo is or comprises a HedgeHog inhibitor (e.g., itraconazole, posaconazole, arsenic trioxide (ATO), Gant61, PF-4708671, HPI-1, HPI-4), a pain inhibitor such as a voltage-gated sodium (Nav) channel inhibitor (e.g., QX-314), and/or an inhibitor of inflammation (e.g., an inhibitor of inflammatory cytokine production, or an NF-kappa B pathway inhibitor).
5. The method of any one of items 1 to 4, wherein the shuttle agent is: (1) a peptide at least 20 amino acids in length comprising (2) an amphipathic alpha-helical motif having (3) a positively-charged hydrophilic outer face, and a hydrophobic outer face, wherein at least five of the following parameters (4) to (15) are respected: (4) the hydrophobic outer face comprises a highly hydrophobic core consisting of spatially adjacent L, I, F, V, W, and/or M amino acids representing 12 to 50% of the amino acids of the peptide, based on an open cylindrical representation of the alpha-helix having 3.6 residues per turn; (5) the peptide has a hydrophobic moment (μ) of 3.5 to 11; (6) the peptide has a predicted net charge of at least +4 at physiological pH; (7) the peptide has an isoelectric point (pI) of 8 to 13; (8) the peptide is composed of 35% to 65% of any combination of the amino acids: A, C, G, I, L, M, F, P, W, Y, and V; (9) the peptide is composed of 0% to 30% of any combination of the amino acids: N, Q, S, and T; (10) the peptide is composed of 35% to 85% of any combination of the amino acids: A, L, K, or R; (11) the peptide is composed of 15% to 45% of any combination of the amino acids: A and L, provided there being at least 5% of L in the peptide; (12) the peptide is composed of 20% to 45% of any combination of the amino acids: K and R; (13) the peptide is composed of 0% to 10% of any combination of the amino acids: D and E; (14) the difference between the percentage of A and L residues in the peptide (% A+L), and the percentage of K and R residues in the peptide (K+R), is less than or equal to 10%; and (15) the peptide is composed of 10% to 45% of any combination of the amino acids: Q, Y, W, P, I, S, G, V, F, E, D, C, M, N, T and H.
6. The method of item 5, wherein: (a) the shuttle agent respects at least six, at least seven, at least eight, at least nine, at least ten, at least eleven, or respects all of parameters (4) to (15); (b) the shuttle agent is a peptide having a minimum length of 20, 21, 22, 23, 24, 25, 26, 27, 28, 29, or 30 amino acids, and a maximum length of 40, 41, 42, 43, 44, 45, 46, 47, 48, 49, 50, 51, 52, 53, 54, 55, 60, 65, 70, 80, 90, 100, 110, 120, 130, 140, or 150 amino acids; (c) said amphipathic alpha-helical motif has a hydrophobic moment (μ) between a lower limit of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, 10.4, 10.5, 10.6, 10.7, 10.8, 10.9, or 11.0; (d) said amphipathic alpha-helical motif comprises a positively-charged hydrophilic outer face comprising: (i) at least two, three, or four adjacent positively-charged K and/or R residues upon helical wheel projection; and/or (ii) a segment of six adjacent residues comprising three to five K and/or R residues upon helical wheel projection, based on an alpha helix having angle of rotation between consecutive amino acids of 100 degrees and/or an alpha-helix having 3.6 residues per turn; (e) said amphipathic alpha-helical motif comprises a hydrophobic outer face comprising: (i) at least two adjacent L residues upon helical wheel projection; and/or (ii) a segment often adjacent residues comprising at least five hydrophobic residues selected from: L, I, F, V, W, and M, upon helical wheel projection, based on an alpha helix having angle of rotation between consecutive amino acids of 100 degrees and/or an alpha-helix having 3.6 residues per turn; (f) said hydrophobic outer face comprises a highly hydrophobic core consisting of spatially adjacent L, I, F, V, W, and/or M amino acids representing from 12.5%, 13%, 13.5%, 14%, 14.5%, 15%, 15.5%, 16%, 16.5%, 17%, 17.5%, 18%, 18.5%, 19%, 19.5%, or 20%, to 25%, 30%, 35%, 40%, or 45% of the amino acids of the shuttle agent; (g) the shuttle agent has a hydrophobic moment (μ) between a lower limit of 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, 10.4, or 10.5; (h) the shuttle agent has a predicted net charge of between +4, +5, +6, +7, +8, +9, to +10, +11, +12, +13, +14, or +15; (i) the shuttle agent has a predicted pI of 10 to 13; or (j) any combination of (a) to (i).
7. The method of any one of items 1 to 6, wherein said shuttle agent respects at least one, at least two, at least three, at least four, at least five, at least six, or all of the following parameters: (8) the shuttle agent is composed of 36% to 64%, 37% to 63%, 38% to 62%, 39% to 61%, or 40% to 60% of any combination of the amino acids: A, C, G, I, L, M, F, P, W, Y, and V; (9) the shuttle agent is composed of 1% to 29%, 2% to 28%, 3% to 27%, 4% to 26%, 5% to 25%, 6% to 24%, 7% to 23%, 8% to 22%, 9% to 21%, or 10% to 20% of any combination of the amino acids: N, Q, S, and T; (10) the shuttle agent is composed of 36% to 80%, 37% to 75%, 38% to 70%, 39% to 65%, or 40% to 60% of any combination of the amino acids: A, L, K, or R; (11) the shuttle agent is composed of 15% to 40%, 20% to 40%, 20 to 35%, or 20 to 30% of any combination of the amino acids: A and L; (12) the shuttle agent is composed of 20% to 40%, 20 to 35%, or 20 to 30% of any combination of the amino acids: K and R; (13) the shuttle agent is composed of 5 to 10% of any combination of the amino acids: D and E; (14) the difference between the percentage of A and L residues in the shuttle agent (% A+L), and the percentage of K and R residues in the shuttle agent (K+R), is less than or equal to 9%, 8%, 7%, 6%, or 5%; and (15) the shuttle agent is composed of 15 to 40%, 20% to 35%, or 20% to 30% of any combination of the amino acids: Q, Y, W, P, I, S, G, V, F, E, D, C, M, N, T, and H.
8. The method of any one of items 1 to 7, wherein said shuttle agent comprises a histidine-rich domain, optionally wherein the histidine-rich domain is: (i) positioned towards the N terminus and/or towards the C terminus of the shuttle agent; (ii) is a stretch of at least 3, at least 4, at least 5, or at least 6 amino acids comprising at least 50%, at least 55%, at least 60%, at least 65%, at least 70%, at least 75%, at least 80%, at least 85%, or at least 90% histidine residues; and/or comprises at least 2, at least 3, at least 4, at least 5, at least 6, at least 7, at least 8, or at least 9 consecutive histidine residues (SEQ ID NO: 368); or (iii) both (i) and (ii).
9. The method of any one of items 1 to 8, wherein said shuttle agent comprises a flexible linker domain rich in serine and/or glycine residues.
10. The method of any one of items 1 to 9, wherein said shuttle agent comprises or consists of the amino acid sequence of (a) [X1]-[X2]-[linker]-[X3]-[X4](Formula 1); (b) [X1]-[X2]-[linker]-[X4]-[X3](Formula 2); (c) [X2]-[X1]-[linker]-[X3]-[X4](Formula 3); (d) [X2]-[X1]-[linker]-[X4]-[X3](Formula 4); (e) [X3]-[X4]-[linker]-[X1]-[X2](Formula 5); (f) [X3]-[X4]-[linker]-[X2]-[X1](Formula 6); (g) [X4]-[X3]-[linker]-[X1]-[X2](Formula 7); or (h) [X4]-[X3]-[linker]-[X2]-[X1](Formula 8), wherein: [X1] is selected from: 2[Φ]-1[+]-2[Φ]-1[ζ]-1[+]-; 2[Φ]-1[+]-2[Φ]-2[+]-; 1[+]-1[Φ]-1[+]-2[c]-1[ζ]-1[+]-; and 1[+]-1[Φ]-1[+]-2[Φ]-2[+]-; [X2] is selected from: -2[Φ]-1[+]-2[Φ]-2[ζ]-; -2[Φ]-1[+]-2[Φ]-2[+]-; -2[(I]-1[+]-2[ζ]-1[+]-I[ζ]-; -2[(I]-1[+]-2[Φ]-1[ζ]-1[+]-; -2[Φ]-2[+]-1[Φ]-2[+]-; -2[Φ]-2[+]-1[Φ]-2[ζ]-; -2[Φ]-2[+]-1[Φ]-1[+]-I[ζ]-; and -2[Φ]-2[+]-1[Φ]-1[ζ]-1[+]-; [X3] is selected from: -4[+]-A-; -3[+]-G-A-; -3[+]-A-A-; -2[+]-1[Φ]-1[+]-A-; -2[+]-1[ζ]-G-A-; -2[+]-1[Φ]-A-A-; or -2[+]-A-1[+]-A; -2[+]-A-G-A; -2[+]-A-A-A-; -1[Φ]-3[+]-A-; -1[Φ]-2[+]-G-A-; -1[Φ]-2[+]-A-A-; -1[Φ]-1[+]-1[ζ]-1[+]-A; -1[Φ]-1[+]-1[ζ]-G-A; -1[Φ]-1[+]-1[ζ]-A-A; -1[ζ]-1[+]-A-1[+]-A; -1[Φ]-1[+]-A-G-A; -1[ζ]-1[+]-A-A-A; -A-1[+]-A-1[+]-A; -A-1[+]-A-G-A (SEQ ID NO: 345); and -A-1[+]-A-A-A (SEQ ID NO: 346); [X4] is selected from: -1[ζ]-2A-1[+]-A; -1[ζ]-2A-2[+]; -1[+]-2A-1[+]-A; -1[ζ]-2A-1[+]-1[ζ]-A-1[+]; -1[ζ]-A-1[ζ]-A-1[+]; -2[+]-A-2[+]; -2[+]-A-1[+]-A; -2[+]-A-1[+]-1[ζ]-A-1[+]; -2[+]-1[ζ]-A-1[+]; -1[+]-1[ζ]-A-1[+]-A; -1[+]-1[ζ]-A-2[+]; -1[+]-1[ζ]-A-1[+]-1[ζ]- A-1[+]; -1[+]-2[ζ]-A-1[+]; -1[+]-2[ζ]-2[+]; -1[+]-2[ζ]-1[+]-A; -1[+]-2[ζ]-1[+]-1[ζ]-A-1[+]; -1[+]-2[ζ]-1[ζ]-A-1[+]; -3[ζ]-2[+]; -3[ζ]-1[+]-A; -3[ζ]-1[+]-1[ζ]-A-1[+]; -1[ζ]-2A-1[+]-A; -1[ζ]-2A-2 [+]; -1[ζ]-2A-1[+]-1[ζ]-A-1 [+]; -2 [+]-A-1 [+]-A; -2[+]-1[ζ]-1[+]-A; -1[+]-1[ζ]-A-1[+]-A; -1[+]-2A-1[+]-1[ζ]-A-1[+]; and -1[ζ]-A-1[ζ]-A-1[+]; and [linker] is selected from: -Gn- (SEQ ID NO: 347); -Sn- (SEQ ID NO: 348); -(GnSn)n- (SEQ ID NO: 349); -(GnSn)nGn-(SEQ ID NO: 350); -(GnSn)nSn- (SEQ ID NO: 351); -(GnSn)nGn(GnSn)n- (SEQ ID NO: 352);
and -(GnSn)nSn(GnSn)n- (SEQ ID NO: 353); wherein: [Φ] is an amino acid which is: Leu, Phe, Trp, Ile, Met, Tyr, or Val, preferably Leu, Phe, Trp, or Ile; [+] is an amino acid which is: Lys or Arg; [ζ] is an amino acid which is: Gln, Asn, Thr, or Ser; A is the amino acid Ala; G is the amino acid Gly; S is the amino acid Ser; and n is an integer from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 1 to 4,or 1 to 3.
11. The method of any one of items 1 to 10, wherein the shuttle agent comprises or consists of: the amino acid sequence any one of SEQ ID NOs: 1 to 50; an amino acid sequence that differs from any one of SEQ ID NOs: 1 to 50 by no more than 1, 2, 3, 4, 5, 6, 7, 8, 9, or 10 amino acids (e.g., excluding any linker domains); or an amino acid sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 1 to 50 (e.g., calculated excluding any linker domains).
12. The method of any one of items 1 to 10, wherein the shuttle agent comprises or consists of: the amino acid sequence any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, or 344; an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, or 344 by no more than 1, 2, 3, 4, 5, 6, 7, 8, 9, or 10 amino acids (e.g., excluding any linker domains); or an amino acid sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 1 to 50, 58 to 78, 80 to 107, 109 to 139, 141 to 146,149 to 161,163 to 169,171,174 to 234, 236 to 240, 242 to 260, 262 to 285, 287 to 294, 296 to 300, 302 to 308, 310, 311, 313 to 324, 326 to 332, 338 to 342, or 344 (e.g., calculated excluding any linker domains).
13. The method of any one of items 1 to 12, wherein the shuttle agent comprises an endosome leakage domain (ELD), and/or a cell penetrating domain (CPD).
14. The method of any one of items 1 to 13, wherein: (i) said ELD is or is from: an endosomolytic peptide; an antimicrobial peptide (AMP); a linear cationic alpha-helical antimicrobial peptide; a Cecropin-A/Melittin hybrid (CM) peptide; pH-dependent membrane active peptide (PAMP); a peptide amphiphile; a peptide derived from the N terminus of the HA2 subunit of influenza hemagglutinin (HA); CM18; Diphtheria toxin T domain (DT); GALA; PEA; INF-7; LAH4; HGP; H5WYG; HA2; EBi; VSVG; Pseudomonas toxin; melittin; KALA; JST-1; C(LLKK)₃C (SEQ ID NO: 366); G(LLKK)₃G (SEQ ID NO: 367); or any combination thereof, (ii) said CPD is or is from: a cell-penetrating peptide or the protein transduction domain from a cell-penetrating peptide; TAT; PTD4; Penetratin; pVEC; M918; Pep-1; Pep-2; Xentry; arginine stretch; transportan; SynB1; SynB3; or any combination thereof; or (iii) both (i) and (ii).
15. The method of any one of items 1 to 14, wherein the shuttle agent is a cyclic peptide and/or comprises one or more D-amino acids.
16. The method of any one of items 1 to 15, wherein the shuttle agent increases the transduction efficiency of propidium iodide or other membrane-impermeable fluorescent DNA intercalating agent by at least 3, 3.5, 4, 4.5, 5, 5.5, 6, 6.5, 7, 7.5, 8, 8.5, 9, 9.5, or 10-fold over a corresponding negative control lacking said shuttle agent, and/or enables a transduction efficiency of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of propidium iodide or other membrane-impermeable fluorescent DNA intercalating agent, in a eukaryotic cell line model (e.g., HeLa) suitable for assessing cargo transduction in said target eukaryotic cells.
17. The method of any one of items 1 to 16, wherein the shuttle agent increases the transduction efficiency of GFP-NLS by at least 3, 3.5, 4, 4.5, 5, 5.5, 6, 6.5, 7, 7.5, 8, 8.5, 9, 9.5, or 10-fold over a corresponding negative control lacking said shuttle agent, and/or enables a transduction efficiency of at least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of GFP-NLS, in a eukaryotic cell line model (e.g., HeLa) suitable for assessing cargo transduction in said target eukaryotic cells.
18. The method of any one of items 1 to 17, wherein the shuttle agent further comprises a chemical modification to one or more amino acids, wherein the chemical modification does not destroy the transduction activity of the synthetic peptide shuttle agent.
19. The method of item 18, wherein the chemical modification is at the N and/or C terminus of the shuttle agent.
20. The method of item 18 or 19, wherein the chemical modification is the addition of an acetyl group (e.g., an N-terminal acetyl group), a cysteamide group (e.g., a C-terminal cysteamide group), or a fatty acid (e.g., C4-C16 fatty acid, preferably N-terminal).
21. The method of any one of items 1 to 20, which is an in vitro method, such as for therapeutic and/or diagnostic purpose.
22. The method of any one of items 1 to 20, which is an in vivo method, such as for therapeutic and/or diagnostic purpose.
23. The method of item 22 comprising topical, enteral/gastrointestinal (e.g., oral), or parenteral administration of the non-proteinaceous cargo and the synthetic peptide shuttle agent.
24. A composition for use in transducing a non-proteinaceous cargo into target eukaryotic cells, the composition comprising a synthetic peptide shuttle agent formulated with a pharmaceutically suitable excipient, wherein the concentration of the synthetic peptide shuttle agent in the composition is sufficient to increase the transduction efficiency and cytosolic and/or nuclear delivery of the non-proteinaceous cargo into said target eukaryotic cells upon administration, as compared to in the absence of said synthetic peptide shuttle agent.
25. The composition of item 24, further comprising the non-proteinaceous cargo.
26. A composition for use in therapy, the composition comprising a synthetic peptide shuttle agent formulated with a non-proteinaceous cargo to be transduced into target eukaryotic cells by the synthetic peptide shuttle agent, wherein the concentration of the synthetic peptide shuttle agent in the composition is sufficient to increase the transduction efficiency and cytosolic and/or nuclear delivery of the non-proteinaceous cargo into said target eukaryotic cells upon administration, as compared to in the absence of said synthetic peptide shuttle agent.
27. The composition of any one of items 24 to 26, wherein: (a) the synthetic peptide shuttle agent is as defined in any one of items 1 or 5 to 20; (b) the non-proteinaceous cargo is as defined in any one of items 2 to 4; (c) the composition is for use in the in vitro or in vivo method as defined in any one of items 21 to 23; or (d) any combination of (a) to (c).
28. A kit for use in the method of any one of items 1 to 23, the kit comprising the synthetic peptide shuttle agent as defined in any one of items 1 or 5 to 20, and the non-proteinaceous cargo is as defined in any one of items 2 to 4.
29. The method of any one of items 1 to 23, the composition of any one of items 24 to 27, or the kit of item 28, wherein the target eukaryotic cells are animal cells, mammalian cells, human cells, stem cells, primary cells, immune cells, T cells, NK cells, dendritic cells, epithelial cells, skin cells, or gastrointestinal cells.
30. A synthetic peptide shuttle agent having transduction activity for both proteinaceous and non-proteinaceous cargoes, the shuttle agent comprising or consisting of: the amino acid sequence any one of SEQ ID NOs: 19 to 50; an amino acid sequence that differs from any one of SEQ ID NOs: 19 to 50 by no more than 1, 2, 3, 4, 5, 6, 7, 8, 9, or 10 amino acids (e.g., excluding any linker domains); or an amino acid sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 19 to 50 (e.g., calculated excluding any linker domains).
31. The synthetic peptide shuttle agent of item 30, which is the shuttle agent as defined in any one of items 5 to 20.
32. A synthetic peptide shuttle agent having transduction activity for both proteinaceous and non-proteinaceous cargoes in target eukaryotic cells, the shuttle agent being: (1) a peptide at least 17, 18, 19, or 20 amino acids in length comprising (2) an amphipathic alpha-helical motif having (3) a positively-charged hydrophilic outer face, and a hydrophobic outer face, wherein at least five of the following parameters (4) to (15) are respected: (4) the hydrophobic outer face comprises a highly hydrophobic core consisting of spatially adjacent L, I, F, V, W, and/or M amino acids representing 12 to 50% of the amino acids of the peptide, based on an open cylindrical representation of the alpha-helix having 3.6 residues per turn; (5) the peptide has a hydrophobic moment (μ) of 3.5 to 11; (6) the peptide has a predicted net charge of at least +4 at physiological pH; (7) the peptide has an isoelectric point (pI) of 8 to 13; (8) the peptide is composed of 35% to 65% of any combination of the amino acids: A, C, G, I, L, M, F, P, W, Y, and V; (9) the peptide is composed of 0% to 30% of any combination of the amino acids: N, Q, S, and T; (10) the peptide is composed of 35% to 85% of any combination of the amino acids: A, L, K, or R; (11) the peptide is composed of 15% to 45% of any combination of the amino acids: A and L, provided there being at least 5% of L in the peptide; (12) the peptide is composed of 20% to 45% of any combination of the amino acids: K and R; (13) the peptide is composed of 0% to 10% of any combination of the amino acids: D and E; (14) the difference between the percentage of A and L residues in the peptide (% A+L), and the percentage of K and R residues in the peptide (K+R), is less than or equal to 10%; and (15) the peptide is composed of 10% to 45% of any combination of the amino acids: Q, Y, W, P, I, S, G, V, F, E, D, C, M, N, T and H, wherein the shuttle agent increases the transduction efficiency of propidium iodide or other membrane-impermeable fluorescent DNA intercalating agent by at least 3, 3.5, 4, 4.5, 5, 5.5, 6, 6.5, 7, 7.5, 8, 8.5, 9, 9.5, or 10-fold over a corresponding negative control lacking said shuttle agent, and/or enables a transduction efficiency of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of propidium iodide or other membrane-impermeable fluorescent DNA intercalating agent, in a eukaryotic cell line model (e.g., HeLa) suitable for assessing cargo transduction in said target eukaryotic cells.
33. The synthetic peptide shuttle agent of item 32, wherein the shuttle agent increases the transduction efficiency of GFP-NLS by at least 3, 3.5, 4, 4.5, 5, 5.5, 6, 6.5, 7, 7.5, 8, 8.5, 9, 9.5, or 10-fold over a corresponding negative control lacking said shuttle agent, and/or enables a transduction efficiency of at least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of GFP-NLS, in a eukaryotic cell line model (e.g., HeLa) suitable for assessing cargo transduction in said target eukaryotic cells.
34. The synthetic peptide shuttle agent of item 32 or 33, wherein: (a) the shuttle agent respects at least six, at least seven, at least eight, at least nine, at least ten, at least eleven, or respects all of parameters (4) to (15); (b) the shuttle agent is a peptide having a minimum length of 17, 18, 19, 20, 21, 22, 23, 24, 25, 26, 27, 28, 29, or 30 amino acids, and a maximum length of 40, 41, 42, 43, 44, 45, 46, 47, 48, 49, 50, 51, 52, 53, 54, 55, 60, 65, 70, 80, 90, 100, 110, 120, 130, 140, or 150 amino acids; (c) said amphipathic alpha-helical motif has a hydrophobic moment (μ) between a lower limit of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, 10.4, 10.5, 10.6, 10.7, 10.8, 10.9, or 11.0; (d) said amphipathic alpha-helical motif comprises a positively-charged hydrophilic outer face comprising: (i) at least two, three, or four adjacent positively-charged K and/or R residues upon helical wheel projection; and/or (ii) a segment of six adjacent residues comprising three to five K and/or R residues upon helical wheel projection, based on an alpha helix having angle of rotation between consecutive amino acids of 100 degrees and/or an alpha-helix having 3.6 residues per turn; (e) said amphipathic alpha-helical motif comprises a hydrophobic outer face comprising: (i) at least two adjacent L residues upon helical wheel projection; and/or (ii) a segment of ten adjacent residues comprising at least five hydrophobic residues selected from: L, I, F, V, W, and M, upon helical wheel projection, based on an alpha helix having angle of rotation between consecutive amino acids of 100 degrees and/or an alpha-helix having 3.6 residues per turn; (f) said hydrophobic outer face comprises a highly hydrophobic core consisting of spatially adjacent L, I, F, V, W, and/or M amino acids representing from 12.5%, 13%, 13.5%, 14%, 14.5%, 15%, 15.5%, 16%, 16.5%, 17%, 17.5%, 18%, 18.5%, 19%, 19.5%, or 20%, to 25%, 30%, 35%, 40%, or 45% of the amino acids of the shuttle agent; (g) the shuttle agent has a hydrophobic moment (μ) between a lower limit of 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, 10.4, or 10.5; (h) the shuttle agent has a predicted net charge of between +4, +5, +6, +7, +8, +9, to +10, +11, +12, +13, +14, or +15; (i) the shuttle agent has a predicted pI of 10 to 13; or (j) any combination of (a) to (i).
35. The synthetic peptide shuttle agent of any one of items 32 to 34, wherein said shuttle agent respects at least one, at least two, at least three, at least four, at least five, at least six, or all of the following parameters: (8) the shuttle agent is composed of 36% to 64%, 37% to 63%, 38% to 62%, 39% to 61%, or 40% to 60% of any combination of the amino acids: A, C, G, I, L, M, F, P, W, Y, and V; (9) the shuttle agent is composed of 1% to 29%, 2% to 28%, 3% to 27%, 4% to 26%, 5% to 25%, 6% to 24%, 7% to 23%, 8% to 22%, 9% to 21%, or 10% to 20% of any combination of the amino acids: N, Q, S, and T; (10) the shuttle agent is composed of 36% to 80%, 37% to 75%, 38% to 70%, 39% to 65%, or 40% to 60% of any combination of the amino acids: A, L, K, or R; (11) the shuttle agent is composed of 15% to 40%, 20% to 40%, 20 to 35%, or 20 to 30% of any combination of the amino acids: A and L; (12) the shuttle agent is composed of 20% to 40%, 20 to 35%, or 20 to 30% of any combination of the amino acids: K and R; (13) the shuttle agent is composed of 5 to 10% of any combination of the amino acids: D and E; (14) the difference between the percentage of A and L residues in the shuttle agent (% A+L), and the percentage of K and R residues in the shuttle agent (K+R), is less than or equal to 9%, 8%, 7%, 6%, or 5%; and (15) the shuttle agent is composed of 15 to 40%, 20% to 35%, or 20% to 30% of any combination of the amino acids: Q, Y, W, P, I, S, G, V, F, E, D, C, M, N, T, and H.
36. The synthetic peptide shuttle agent of any one of items 32 to 35, wherein said shuttle agent: (i) comprises a histidine-rich domain as defined in item 8; (ii) comprises a flexible linker domain as defined in item 9; (iii) is the shuttle agent as defined in any one of items 10 to 14; or (iv) any combination of (i) to (iii).
37. The synthetic peptide shuttle agent of any one of items 32 to 36, further comprising a chemical modification to one or more amino acids, wherein the chemical modification does not destroy the transduction activity of the synthetic peptide shuttle agent.
38. The synthetic peptide shuttle agent of item 37, wherein the chemical modification is at the N and/or C terminus of the shuttle agent.
39. The synthetic peptide shuttle agent of item 37 or 38, wherein the chemical modification is the addition of an acetyl group (e.g., an N-terminal acetyl group), a cysteamide group (e.g., a C-terminal cysteamide group), or a fatty acid (e.g., C4-C16 fatty acid, preferably N-terminal).
40. The synthetic peptide shuttle agent of any one of items 32 to 39, wherein the shuttle agent comprises or consists of: the amino acid sequence any one of SEQ ID NOs: 1 to 50, 58 to 78, 80 to 107, 109 to 139, 141 to 146,149 to 161,163 to 169,171,174 to 234, 236 to 240, 242 to 260, 262 to 285, 287 to 294, 296 to 300, 302 to 308, 310, 311, 313 to 324, 326 to 332, 338 to 342, or 344; an amino acid sequence that differs from any one of SEQ ID NOs: 1 to 50, 58 to 78, 80 to 107, 109 to 139, 141 to 146, 149 to 161, 163 to 169,171,174 to 234, 236 to 240, 242 to 260, 262 to 285, 287 to 294, 296 to 300, 302 to 308, 310, 311, 313 to 324, 326 to 332, 338 to 342, or 344 by no more than 1, 2, 3, 4, 5, 6, 7, 8, 9, or 10 amino acids (e.g., excluding any linker domains); or an amino acid sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 1 to 50, 58 to 78, 80 to 107,109 to 139,141 to 146,149 to 161,163 to 169,171,174 to 234, 236 to 240, 242 to 260, 262 to 285, 287 to 294, 296 to 300, 302 to 308, 310, 311, 313 to 324, 326 to 332, 338 to 342, or 344 (e.g., calculated excluding any linker domains).
41. A synthetic peptide shuttle agent having transduction activity for both proteinaceous and non-proteinaceous cargoes in target eukaryotic cells, wherein the shuttle agent comprises or consists of: (a) the amino acid sequence any one of SEQ ID NOs: 1 to 50, 58 to 78, 80 to 107, 109 to 139, 141 to 146, 149 to 161, 163 to 169,171,174 to 234, 236 to 240, 242 to 260, 262 to 285, 287 to 294, 296 to 300, 302 to 308, 310, 311, 313 to 324, 326 to 332, 338 to 342, or 344; or (b) an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, or 344 by only conservative amino acid substitutions (e.g., by no more than no more than 1, 2, 3, 4, 5, 6, 7, 8, 9, or 10 conservative amino acid substitutions, preferably excluding any linker domains), wherein shuttle agent: increases the transduction efficiency of propidium iodide or other membrane-impermeable fluorescent DNA intercalating agent by at least 3, 3.5, 4, 4.5, 5, 5.5, 6, 6.5, 7, 7.5, 8, 8.5, 9, 9.5, or 10-fold over a corresponding negative control lacking said shuttle agent; and/or enables a transduction efficiency of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of propidium iodide or other membrane-impermeable fluorescent DNA intercalating agent, in a eukaryotic cell line model (e.g., HeLa) suitable for assessing cargo transduction in said target eukaryotic cells.
42. The synthetic peptide shuttle agent of item 41, which is the synthetic peptide shuttle agent as defined in any one of items 32 to 39.
43. A synthetic peptide shuttle agent having proteinaceous cargo transduction activity in target eukaryotic cells, wherein the shuttle agent comprises or consists of: (a) the amino acid sequence any one of SEQ ID NOs: 52, 57, 79, 108, 140, 147, 148, 173, 241, 261, 286, 295, 301, 309, 312, 325, 333-337, or 343; or (b) an amino acid sequence that differs from any one of SEQ ID NOs: 52, 57, 79, 108, 140, 147, 148, 173, 241, 261, 286, 295, 301, 309, 312, 325, 333-337, or 343 by only conservative amino acid substitutions (e.g., by no more than no more than 1, 2, 3, 4, 5, 6, 7, 8, 9, or 10 conservative amino acid substitutions, preferably excluding any linker domains), wherein shuttle agent: increases the transduction efficiency of GFP-NLS by at least 3, 3.5, 4, 4.5, 5, 5.5, 6, 6.5, 7, 7.5, 8, 8.5, 9, 9.5, or 10-fold over a corresponding negative control lacking said shuttle agent, and/or enables a transduction efficiency of at least 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% (e.g., as determined by flow cytometry) of GFP-NLS in a eukaryotic cell line model (e.g., HeLa) suitable for assessing cargo transduction in said target eukaryotic cells.
44. The synthetic peptide shuttle agent of any one of items 41 to 43, wherein each conservative amino acid substitution is selected from an amino acid within the same amino acid class, the amino acid class being: Aliphatic: G, A, V, L, and I; Hydroxyl or sulfur/selenium-containing: S, C, U, T, and M; Aromatic: F, Y, and W; Basic: H, K, and R; Acidic and their amides: D, E, N, and Q.
45. A synthetic peptide shuttle agent variant having transduction activity for proteinaceous and/or non-proteinaceous cargoes in target eukaryotic cells, the synthetic peptide shuttle agent variant being identical to the synthetic peptide shuttle agent as defined in any one of items 32 to 44, except having at least one amino acid being replaced with a corresponding synthetic amino acid having a side chain of similar physiochemical properties (e.g., structure, hydrophobicity, or charge) as the amino acid being replaced, wherein the shuttle agent variant increases the transduction efficiency of said cargo in the target eukaryotic cells, as compared to in the absence of the shuttle agent variant.
46. The synthetic peptide shuttle agent variant of item 45, wherein the synthetic amino acid replacement:
   (a) replaces a basic amino acids with any one of: α-aminoglycine, α,γ-diaminobutyric acid, ornithine, α,β-diaminopropionic acid, 2,6-diamino-4-hexynoic acid, β-(1-piperazinyl)-alanine, 4,5-dehydro-lysine, δ-hydroxylysine, ω,ω-dimethylarginine, homoarginine, ω,ω′-dimethylarginine, ω-methylarginine, β-(2-quinolyl)-alanine, 4-aminopiperidine-4-carboxylic acid, α-methylhistidine, 2,5-diiodohistidine, 1-methylhistidine, 3-methylhistidine, spinacine, 4-aminophenylalanine, 3-aminotyrosine, β-(2-pyridyl)-alanine, or β-(3-pyridyl)-alanine;
   (b) replaces a non-polar (hydrophobic) amino acid with any one of: dehydro-alanine, β-fluoroalanine, β-chloroalanine, β-lodoalanine, α-aminobutyric acid, α-aminoisobutyric acid, β-cyclopropylalanine, azetidine-2-carboxylic acid, α-allylglycine, propargylglycine, tert-butylalanine, β-(2-thiazolyl)-alanine, thiaproline, 3,4-dehydroproline, tert-butylglycine, β-cyclopentylalanine, β-cyclohexylalanine, α-methylproline, norvaline, α-methylvaline, penicillamine, β, β-dicyclohexylalanine, 4-fluoroproline, 1-aminocyclopentanecarboxylic acid, pipecolic acid, 4,5-dehydroleucine, allo-isoleucine, norleucine, α-methylleucine, cyclohexylglycine, cis-octahydroindole-2-carboxylic acid, β-(2-thienyl)-alanine, phenylglycine, α-methylphenylalanine, homophenylalanine, 1,2,3,4-tetrahydroisoquinoline-3-carboxylic acid, β-(3-benzothienyl)-alanine, 4-nitrophenylalanine, 4-bromophenylalanine, 4-tert-butylphenylalanine, α-methyltryptophan, β-(2-naphthyl)-alanine, β-(1-naphthyl)-alanine, 4-iodophenylalanine, 3-fluorophenylalanine, 4-fluorophenylalanine, 4-methyltryptophan, 4-chlorophenylalanine, 3,4-dichloro-phenylalanine, 2,6-difluoro-phenylalanine, n-in-methyltryptophan, 1,2,3,4-tetrahydronorharman-3-carboxylic acid, β,β-diphenylalanine, 4-methylphenylalanine, 4-phenylphenylalanine, 2,3,4,5,6-pentafluoro-phenylalanine, or 4-benzoylphenylalanine;
   (c) replaces a polar, uncharged amino acid with any one of β-cyanoalanine, β-ureidoalanine, homocysteine, allo-threonine, pyroglutamic acid, 2-oxothiazolidine-4-carboxylic acid, citrulline, thiocitrulline, homocitrulline, hydroxyproline, 3,4-dihydroxyphenylalanine, β-(1,2,4-triazol-1-yl)-alanine, 2-mercaptohistidine, β-(3,4-dihydroxyphenyl)-serine, β-(2-thienyl)-serine, 4-azidophenylalanine, 4-cyanophenylalanine, 3-hydroxymethyltyrosine, 3-iodotyrosine, 3-nitrotyrosine, 3,5-dinitrotyrosine, 3,5-dibromotyrosine, 3,5-diiodotyrosine, 7-hydroxy-1,2,3,4-tetrahydroiso-quinoline-3-carboxylic acid, 5-hydroxytryptophan, thyronine, B-(7-methoxycoumarin-4-yl)-alanine, or 4-(7-hydroxy-4-coumarinyl)-aminobutyric acid; and/or
   (d) replaces an acidic amino acid with any one of γ-hydroxyglutamic acid, γ-methyleneglutamic acid, γ-carboxyglutamic acid, α-aminoadipic acid, 2-aminoheptanedioic acid, α-aminosuberic acid, 4-carboxyphenylalanine, cysteic acid, 4-phosphonophenylalanine, or 4-sulfomethylphenylalanine.
47. The synthetic peptide shuttle agent or synthetic peptide shuttle agent variant as defined in any one of items 32 to 46 for use in an in vitro or in vivo method for increasing the transduction efficiency of a proteinaceous and/or non-proteinaceous cargo (e.g., a therapeutically active proteinaceous and/or non-proteinaceous cargo) into target eukaryotic cells, wherein the synthetic peptide shuttle agent or synthetic peptide shuttle agent variant is used at a concentration sufficient to increase the transduction efficiency and cytosolic and/or nuclear delivery of the cargo into the target eukaryotic cells, as compared to in the absence of the synthetic peptide shuttle agent or synthetic peptide shuttle agent variant.
48. The synthetic peptide shuttle agent or synthetic peptide shuttle agent variant as defined in any one of items 32 to 47 for use in therapy, wherein the synthetic peptide shuttle agent or synthetic peptide shuttle agent variant transduces a therapeutically active proteinaceous and/or non-proteinaceous cargo to the cytosol and/or nucleus of target eukaryotic cells, wherein the synthetic peptide shuttle agent or synthetic peptide shuttle agent variant is used at a concentration sufficient to increase the transduction efficiency of the cargo into the target eukaryotic cells, as compared to in the absence of the synthetic peptide shuttle agent.
49. An in vitro or in vivo method for proteinaceous and/or non-proteinaceous cargo transduction, the method comprising contacting target eukaryotic cells with the cargo and a concentration of the synthetic peptide shuttle agent or synthetic peptide shuttle agent variant as defined in any one of items 32 to 46 sufficient to increase the transduction efficiency of the cargo into the target eukaryotic cells, as compared to in the absence of said synthetic peptide shuttle agent.
50. The in vitro or in vivo method of item 49, which is a method for therapeutic and/or diagnostic purpose.
51. A composition for use in therapy, the composition comprising the synthetic peptide shuttle agent or synthetic peptide shuttle agent variant as defined in any one of items 32 to 46 formulated with a proteinaceous and/or non-proteinaceous cargo to be transduced into target eukaryotic cells by the synthetic peptide shuttle agent, wherein the concentration of the synthetic peptide shuttle agent or synthetic peptide shuttle agent variant in the composition is sufficient to increase the transduction efficiency and cytosolic delivery of the cargo into said target eukaryotic cells upon administration, as compared to in the absence of said synthetic peptide shuttle agent.
52. The composition of item 51, which is formulated for topical, enteral/gastrointestinal (e.g., oral), or parenteral administration.
53. A kit comprising the synthetic peptide shuttle agent or synthetic peptide shuttle agent variant as defined in any one of items 32 to 46, and a proteinaceous and/or non-proteinaceous cargo to be transduced by the synthetic peptide shuttle agent or synthetic peptide shuttle agent variant.
54. The synthetic peptide shuttle agent or synthetic peptide shuttle agent variant of any one of items 32 to 48, the in vitro or in vivo method of item 49 or 50, the composition of item 51 or 52, or the kit of item 53, wherein the target eukaryotic cells are animal cells, mammalian cells, human cells, stem cells, primary cells, immune cells, T cells, NK cells, dendritic cells, epithelial cells, skin cells, or gastrointestinal cells.
55. The synthetic peptide shuttle agent or synthetic peptide shuttle agent variant of any one of items 32 to 48 or 54, the in vitro or in vivo method of item 49 or 50 or 54, the composition of item 51, 52, or 54, or the kit of item 53 or 54, wherein the non-proteinaceous cargo is as defined in any one of items 2 to 4.
56. A process for producing a candidate synthetic peptide shuttle agent expected to have transduction activity for a cargo of interest in target eukaryotic cells, the method comprising synthesizing a peptide which is: (1) a peptide at least 17, 18, 19, or 20 amino acids in length comprising (2) an amphipathic alpha-helical motif having (3) a positively-charged hydrophilic outer face, and a hydrophobic outer face, wherein at least five of the following parameters (4) to (15) are respected: (4) the hydrophobic outer face comprises a highly hydrophobic core consisting of spatially adjacent L, I, F, V, W, and/or M amino acids representing 12 to 50% of the amino acids of the peptide, based on an open cylindrical representation of the alpha-helix having 3.6 residues per turn; (5) the peptide has a hydrophobic moment (μ) of 3.5 to 11; (6) the peptide has a predicted net charge of at least +4 at physiological pH; (7) the peptide has an isoelectric point (pI) of 8 to 13; (8) the peptide is composed of 35% to 65% of any combination of the amino acids: A, C, G, I, L, M, F, P, W, Y, and V; (9) the peptide is composed of 0% to 30% of any combination of the amino acids: N, Q, S, and T; (10) the peptide is composed of 35% to 85% of any combination of the amino acids: A, L, K, or R; (11) the peptide is composed of 15% to 45% of any combination of the amino acids: A and L, provided there being at least 5% of L in the peptide; (12) the peptide is composed of 20% to 45% of any combination of the amino acids: K and R; (13) the peptide is composed of 0% to 10% of any combination of the amino acids: D and E; (14) the difference between the percentage of A and L residues in the peptide (% A+L), and the percentage of K and R residues in the peptide (K+R), is less than or equal to 10%; and (15) the peptide is composed of 10% to 45% of any combination of the amino acids: Q, Y, W, P, I, S, G, V, F, E, D, C, M, N, T and H, wherein the shuttle agent increases the transduction efficiency of propidium iodide or other membrane-impermeable fluorescent DNA intercalating agent by at least 3, 3.5, 4, 4.5, 5, 5.5, 6, 6.5, 7, 7.5, 8, 8.5, 9, 9.5, or 10-fold over a corresponding negative control lacking said shuttle agent, and/or enables a transduction efficiency of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of propidium iodide or other membrane-impermeable fluorescent DNA intercalating agent, in a eukaryotic cell line model (e.g., HeLa) suitable for assessing cargo transduction in said target eukaryotic cells.
57. The process of item 56, wherein the candidate synthetic peptide shuttle agent is the synthetic peptide shuttle agent or synthetic peptide shuttle agent variant as defined in any one of items 32 to 46.
58. An in vitro or in vivo method for identifying, selecting, or qualifying a synthetic peptide shuttle agent expected to have transduction activity for both proteinaceous and non-proteinaceous cargoes in target eukaryotic cells, the method comprising: providing model eukaryotic cells or a model organism suitable for assessing cargo transduction in the target eukaryotic cells; providing a candidate synthetic peptide shuttle agent (e.g., as defined in any one of items 5 to 20 or 32 to 46); and measuring the transduction activity (e.g., cargo transduction efficiency, such as by flow cytometry) of the candidate synthetic peptide shuttle agent to transduce propidium iodide or other membrane-impermeable fluorescent DNA intercalating agent into the model eukaryotic cells or model organism, wherein the candidate shuttle agent is expected to have transduction activity for both proteinaceous and non-proteinaceous cargoes in the target eukaryotic cells when the transduction activity (e.g., transduction efficiency) of propidium iodide or other membrane-impermeable fluorescent DNA intercalating agent is increased by at least 3, 3.5, 4, 4.5, 5, 5.5, 6, 6.5, 7, 7.5, 8, 8.5, 9, 9.5, or 10-fold over a corresponding negative control lacking the candidate synthetic peptide shuttle agent, and/or a transduction efficiency of at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% (e.g., as determined by flow cytometry) of the propidium iodide or other membrane-impermeable fluorescent DNA intercalating agent occurs, in the model eukaryotic cells or model organism.

### EXAMPLES

### Example 1: Materials and Methods

All materials and methods not described or specified herein were generally as performed in WO/2016/161516 and/or WO/2018/068135.

1.1 Materials and reagents

| Material | Company | City, Province-State, Country |
|---|---|---|
| RPMI 1640 media | Sigma-Aldrich | Oakville, ON, Canada |
| DMEM | Sigma-Aldrich | Oakville, ON, Canada |
| Alpha MEM | Stem Cell Technology | Oakville, ON, Canada |
| Fetal bovine serum (FBS) | NorthBio | Toronto, ON, Canada |
| Geneticin | VWR/100218-044 | Ville Mont-Royal, QC, Canada |
| Non-essential amino acids | VWR/10128-762 | Ville Mont-Royal, QC, Canada |
| Na-pyruvate | VWR/CAAAJ61840-18 | Ville Mont-Royal, QC, Canada |
| HEPES | VWR/CA97061-824 | Ville Mont-Royal, QC, Canada |
| L-glutamine-Penicillin-Streptomycin | Sigma-Aldrich | Oakville, ON, Canada |
| Trypsin-EDTA solution | Sigma-Aldrich | Oakville, ON, Canada |
| Dexamethasone | Sigma-Aldrich | Oakville, ON, Canada |
| Cyto Tox-ONE | Promega | Madison, Wisconsin, United States |
| DMSO | Sigma-Aldrich/D2650-100 ml | Oakville, ON, Canada |
| Itraconazole | VWR/10188-660 | Ville Mont-Royal, QC, Canada |
| Gant61 | Santa Cruz | Dallas, Texas, United States |
| | Biotechnology/SC-202630 | |
| HPI4 | Cedarlane/A16349-10 | Burlington, ON, Canada |
| Arsenic trioxide (ATO) | VWR/CAAA33289-14 | Ville Mont-Royal, QC, Canada |
| Recombinant mouse Sonic HedgeHog | Genscript/Z03050 | Piscataway, NJ, United States |
| (mShh) | | |
| ONE-Step Luciferase Assay kit | BPS Bioscience/60690-1 | San Diego, CA |
| Propidium iodide (PI) | Sigma-Aldrich/P4170-10 MG | Oakville, ON, Canada |
| HisPrep ™ column | GE Healthcare | Baie d'Urfe, QC, Canada |
| Q Sepharose ™ | GE Healthcare | Baie d'Urfe, QC, Canada |
| Amicon Ultra centrifugal filters | EMD Millipore | Etobicoke, ON Canada |
| Resazurin sodium salt | Sigma-Aldrich/R7017-1G | Oakville, ON, Canada |
| PES syringe filter 0.2 um | VWR/28145-501 | Ville Mont-Royal, QC, Canada |
| Alexa ™-594 Anti-Mouse | Abcam #150116 | Toronto, ON, Canada |
| Fluoroshield ™ with DAPI | Sigma #F6057 | Oakville, ON, Canada |
| Phusion ™ High-Fidelity DNA polymerase | (NEB #M0530S) | Whitby, ON, Canada |
| Opti-MEM ™ | Sigma-Aldrich | Oakville, ON, Canada |
| QX-314 | Sigma Aldrich/L5783- | Oakville, ON, Canada |
| | 250 MG | |

### 1.3 Cell Lines and Culture Conditions

Cells were cultured following the manufacturer's instructions.

| | | | Culture | | |
|---|---|---|---|---|---|
| Cell lines | Description | ATCC/others | media | Serum | Additives |
| HeLa | Human cervical | ATCC ™ CCL-2 | DMEM | 10% FBS | L-glutamine 2 mM |
| | carcinoma cells | | | | Penicillin 100 units |
| | | | | | Streptomycin 100 μg/mL |
| NIH3T3 | Mouse Swiss | BPS | DMEM | 10% BCS | 1% Pen/Strep |
| Gli-luciferase | NIH embryo | | | | 500 μg/ml Geneticin |
| cells | fibroblasts | Bioscience/60409 | Opti-MEM | 0.5% BCS | 1% Non essential amino |
| | | | | | acids |
| | | | | | 1mM Na-pyruvate |
| | | | | | 10mM HEPES |
| | | | | | 1% Pen/Strep |
| HEK293 cells | Human | Theriault et al., | DMEM | 10% FBS | L-glutamine 2 mM |
| | embryonic | 2015 | | | penicillin 100 U/mL |
| | kidney 293 cells | | | | Streptomycin 10 mg/mL |
| | modified to | | | | |
| | express Nav1.7 | | | | |

FBS: Fetal bovine serum
BCS: Bovine calf serum

### 1.4 Propidium Iodide Transduction Protocol

HeLa cells were plated (20 000 cells/well) in a 96 well-dish the day prior the experiment. Each delivery mix comprising a synthetic peptide shuttle agent (10 μM) and the propidium iodide (PI) (10 μg/mL) or the GFP-NLS (10 μM) were prepared and completed to 50 μL with phosphate-buffered saline (PBS). Cells were washed once with PBS and the Shuttle/PI or Shuttle/GFP-NLS added on cells for one minute. Then 100 μL DMEM containing 10% FBS was added to the mix and removed. Cells were washed once with PBS and incubated in DMEM containing 10% FBS. Cells were analyzed after 2-hour incubation by flow cytometry. For the condition “FS then PI”, only the synthetic peptide shuttle agent (10 μM) was added on HeLa cells for 1 minute and one hour later PI (10 μg/mL) was added for one minute following the same washing step. Cells were analyzed one hour after PI or GFP-NLS treatment.

### 1.5 Hedgehog Pathway Inhibitors Transduction Protocol in Gli Reporter NIH3T3 Cells

Stock solutions of cargoes were prepared as follows: Gant61 stock (20 mM in DMSO); HPI4 stock (40 mM in DMSO); Itraconazole stock (4.8 mM (4 mg/mL) in DMSO); Arsenic trioxide (ATO) stock (40 mM in H₂O). Peptide shuttle agent (5 μM) and Hedgehog pathway inhibitor (100 μM) were mixed and volume was completed to 50 μL with PBS.

Hedgehog signaling pathway Gli Reporter NIH 3T3 cells were cultured in DMEM containing 10% calf serum. Cells were trypsinized, centrifuged and resuspended at 10 million cells/mL in PBS. 50 μL of cells (500 000 cells/well) were distributed in a round bottom non-treated 96-well plate. Resuspended cells were mixed with a delivery mix containing the peptide shuttle agent (5 μM) and Hedgehog pathway inhibitor (100 μM). Cells were incubated 90 seconds with the delivery mix at room temperature, 200 μL of DMEM containing 10% calf serum (200 μL) was added in each well, and cells were centrifuged (400 g, 4 min.) and washed with 200 μL of PBS. Cells were then resuspended in 200 μL of DMEM and then transferred to a well of a 6-well plate containing 1 mL of DMEM containing 10% calf serum and incubated at 37° C. for 2 hours. The media was gently removed and 1 ml of either control media (Opti-MEM™) or activating media (Opti-MEM with 5 μg/mL mShh) was added to each well. Cells were incubated at 37° C. for 24-30 hours.

For analyses, cells were trypsinized and resuspended in each well with 200 μL of Opti-MEM™, and then split equally to two wells of a round bottom 96-well plate. Viability was assessed using flow cytometry analysis and ONE-Step Luciferase assay was used to measure luminescence following manufacturer's instructions.

### 1.6 Hedgehog Pathway Inhibitors Transduction Protocol In Vivo

Cargoes were suspended as recommended: Gant61 stock 20 mM in DMSO; Itraconazole stock 4.8 mM (4 mg/mL) in DMSO. Female C57BL6 mice aged between 6 to 7 weeks were shaved and depilated using hair removal product (Nair™). Five days after depilation, 30 μL of a mix containing PBS, the synthetic peptide shuttle agent FSD250D (SEQ ID NO: 36), and/or the cargo were applied on 3 cm² of the depilated skin. Mice were imaged 3, 10 and 17 days after treatment.

### 1.7 QX-314 and GFP-NLS Co-Transduction and Patch-Clamp Technique

Cell culture. HEK293 cells stably expressing Nav1.7 were grown in Dulbecco's minimal essential medium (DMEM, Gibco BRL Life Technologies) supplemented with fetal bovine serum (FBS, 10%), L-glutamine (2 mM), penicillin (100 U/mL), streptomycin (10 mg/mL). The cells were incubated at 37° C. in a 5% CO₂ humidified atmosphere.

Delivery. Cells were seeded 24 hours prior to the experiment in 24-well plate. Cells were washed twice with PBS. A solution containing 1 mM of QX-314, 5 μM of FSD194 and 15 μM of GFP-NLS protein was applied to cells for 90 seconds and removed by aspiration. Control was performed using 5 μM of FSD194 or of 2.5 mM QX-314, in presence of GFP-NLS. Cells were washed with 800 μL of DMEM containing 10% FBS and transferred in the recording solution to perform the electrophysiology. GFP-positive cells were determined by microscopy and then selected for patch-clamp analysis.

Electrophysiology. Frequency protocol with QX-314 was recorded within 30 s after the whole-cell configuration was formed. Frequency protocol consists of a pulse of 10 ms at −20 mV from a holding potential of −140 mV at 10 Hz. Whole-cell Na+ currents in HEK293 cells were recorded at room temperature using an Axopatch 200B with the whole-cell configuration of the patch-clamp technique (Molecular Devices). pClamp v10.0 was used for the pulse stimulations and recordings (Molecular Devices). Currents were filtered at 5 kHz, digitized at 100 kHz using a Digidata 1550 AD converter (Molecular devices), and stored on a computer for subsequent analyses. Series resistance was compensated by 70-80%. When needed, linear leak current artifacts were removed using on-line leak subtraction. Fire-polished low-resistance electrodes (2MΩ) were pulled from 8161 glass (Corning).

Recording Solutions. Bath solution: 35 mM NaCl, 115 mM NMDG, 2 mM KCl, 1.5 mM CaCl₂, 1.0 mM MgCl₂, 10 mM glucose, 10 mM HEPES. The pH was adjusted to pH 7.3 with 1 M NaOH. Pipette solution: 35 mM NaCl, 105 mM CsF, 10 mM EGTA, and 10 mM HEPES. The pH was adjusted to pH 7.4 with 1 M CsOH.

### Example 2: Synthetic Peptide Shuttle Agents Enable Intracellular Delivery of Propidium Iodide

Propidium iodide (PI) is a fluorescent DNA intercalating dye often used as a nuclear stain in fluorescence microscopy and flow cytometry applications. Binding of PI to DNA results in enhanced fluorescence by 20- to 30-fold, as well as a shift in its maximum excitation/emission spectra. Since PI is not normally able to cross the plasma membrane of live cells, it is routinely used to detect dead cells in a cell population. It was surprisingly found herein that synthetic peptide shuttle agents, including shuttle agent peptides described in WO/2016/161516 and WO/2018/068135 for the transduction of proteinaceous cargoes, are able to transduce PI as well as other non-proteinaceous cargoes.

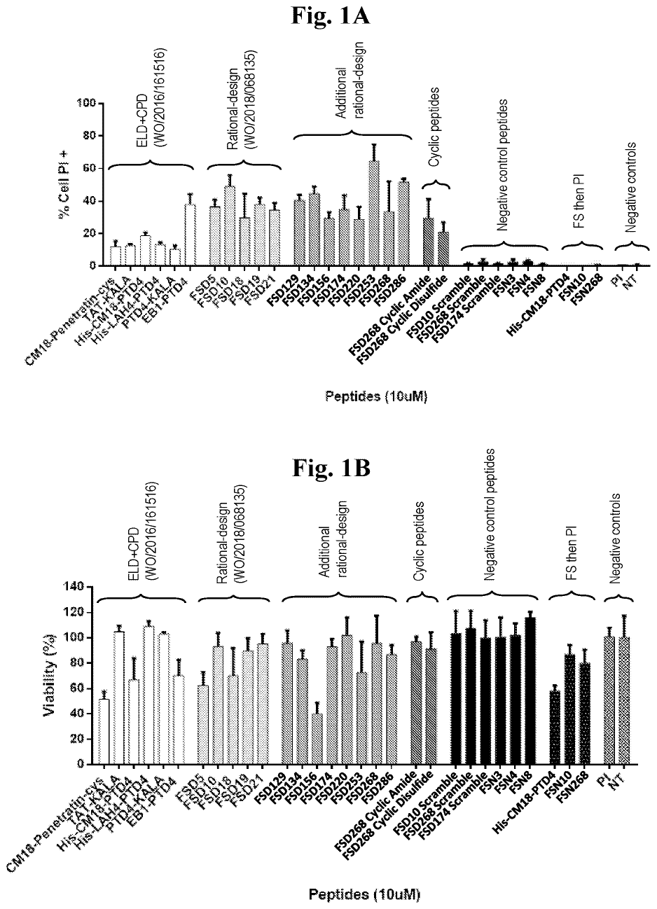

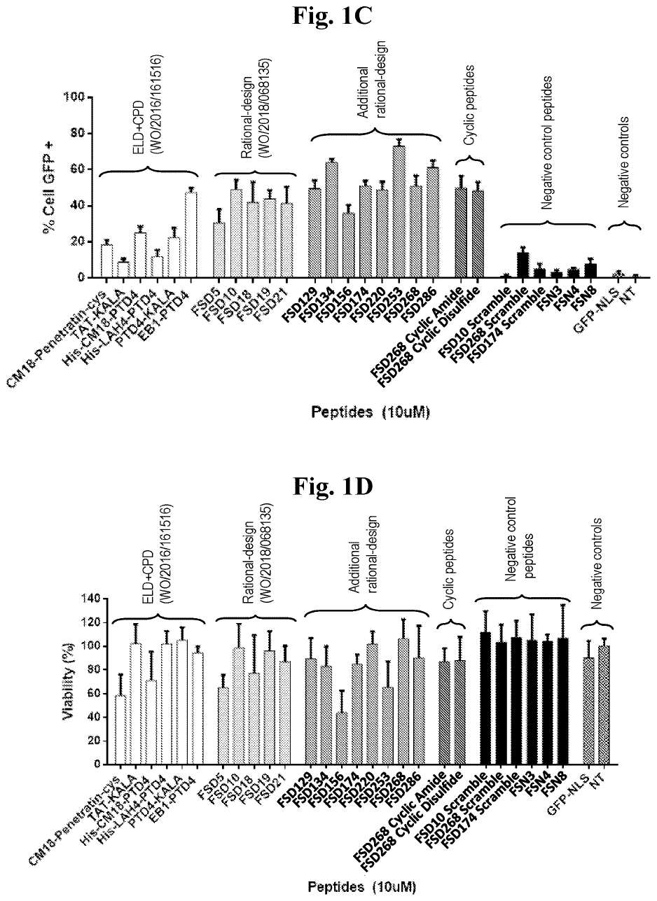

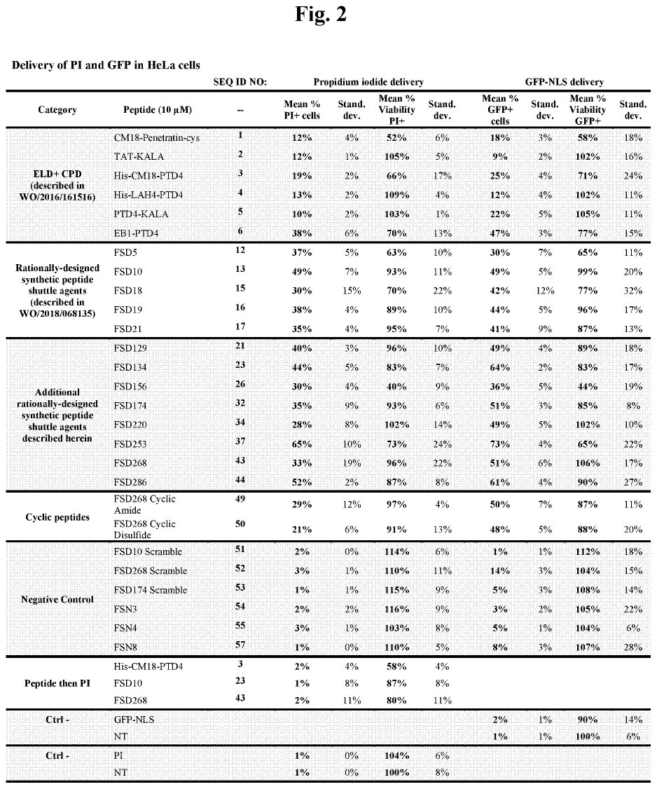

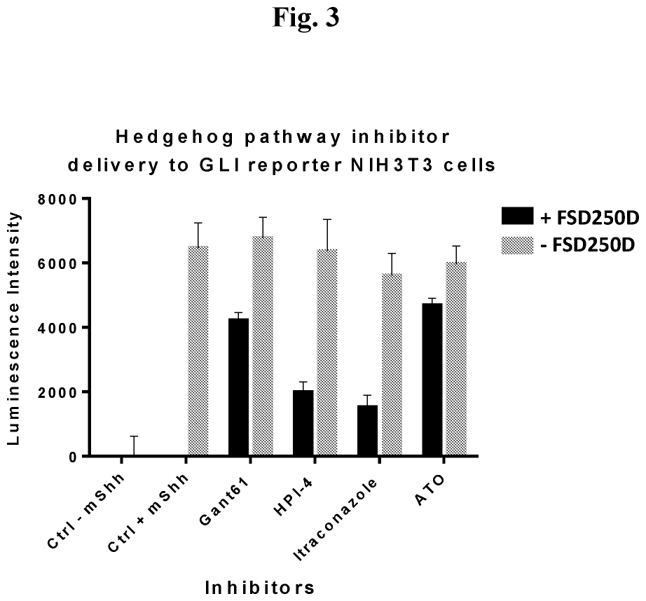

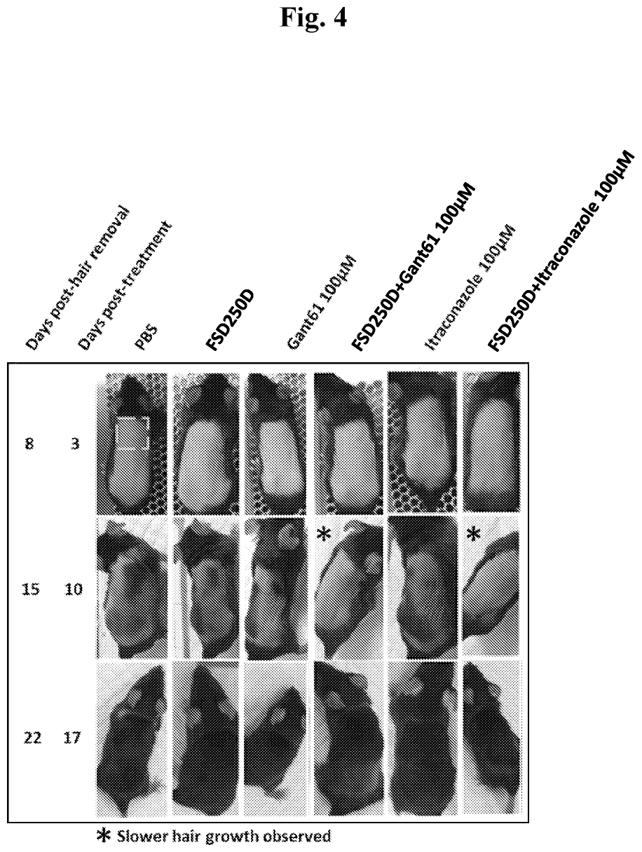

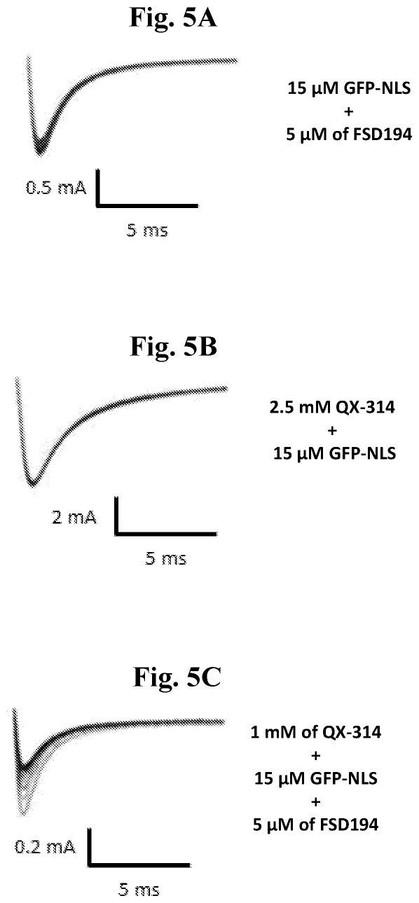

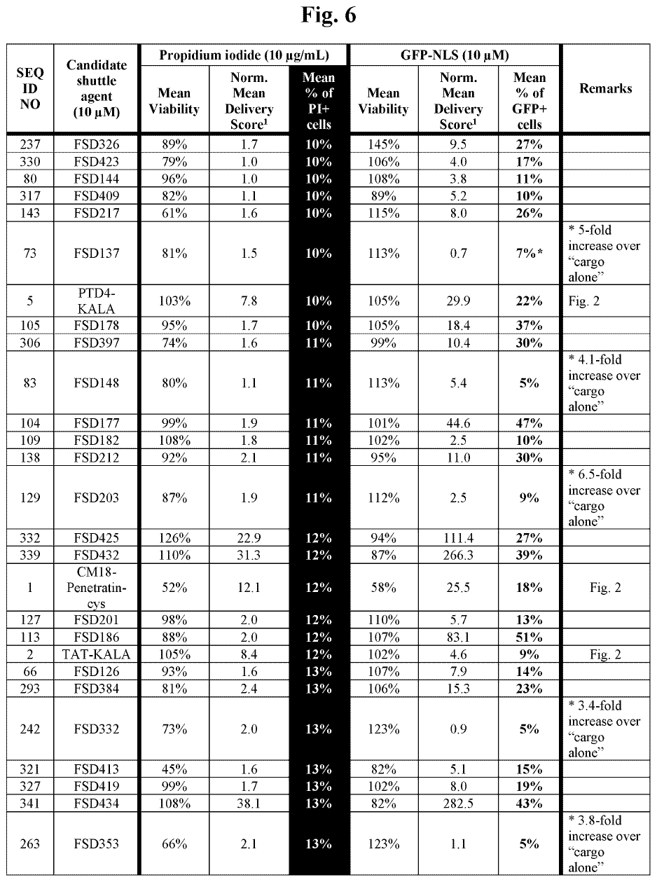

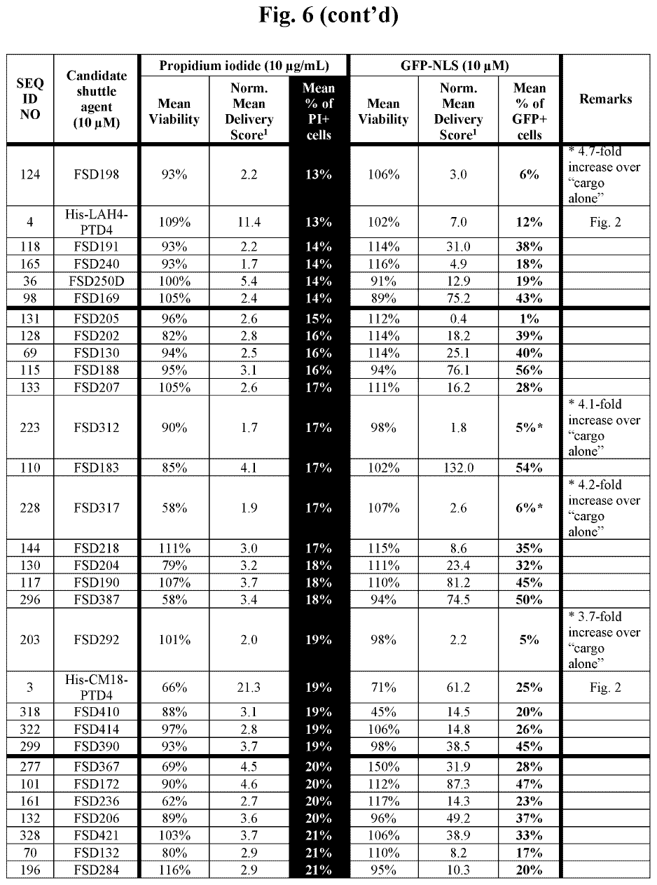

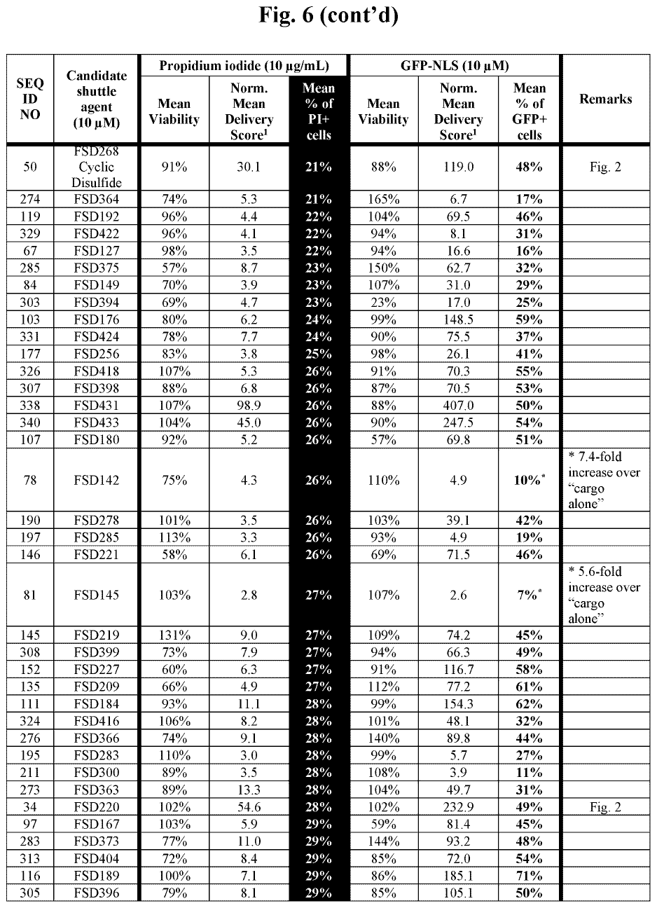

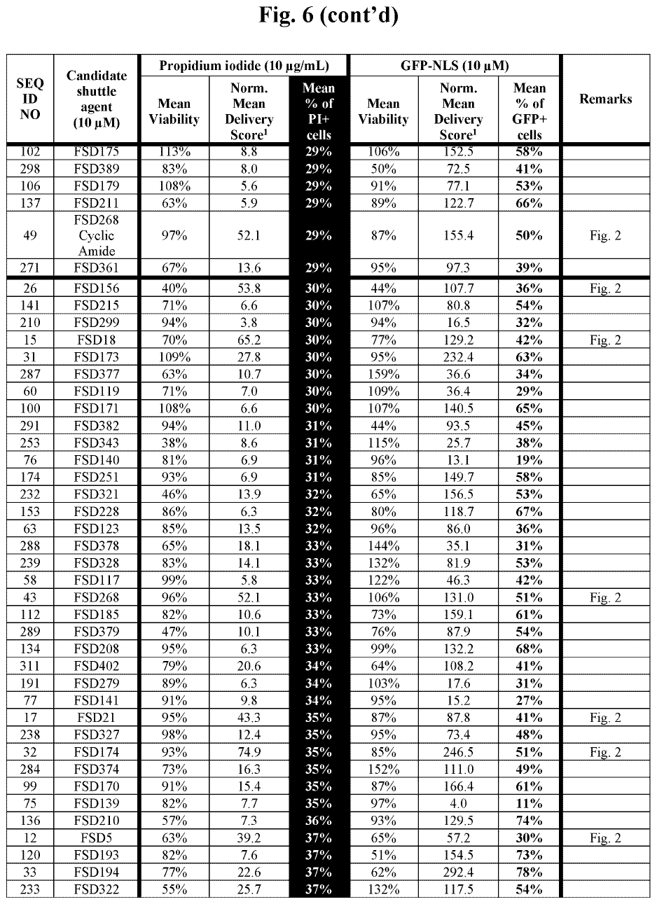

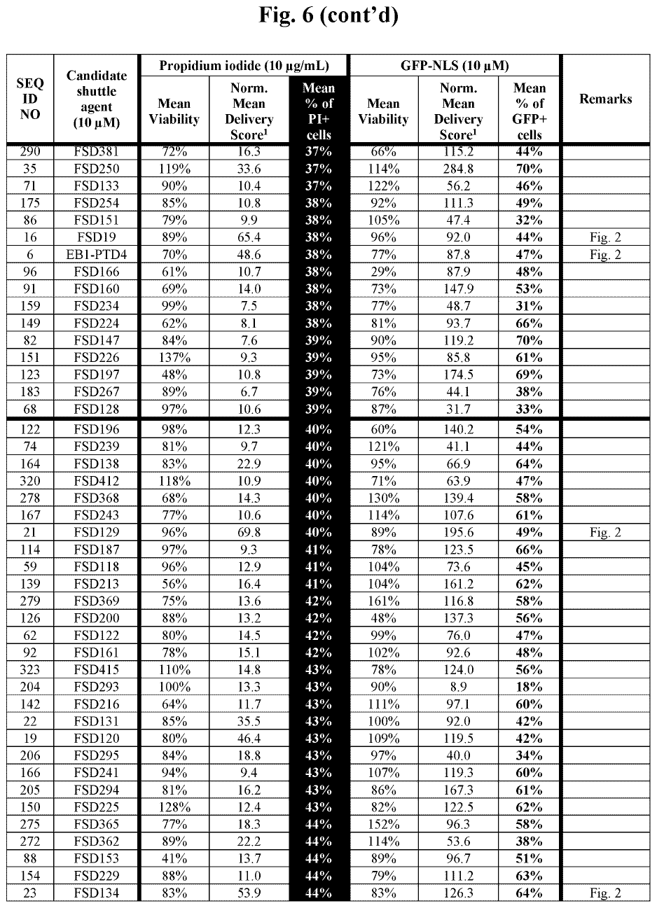

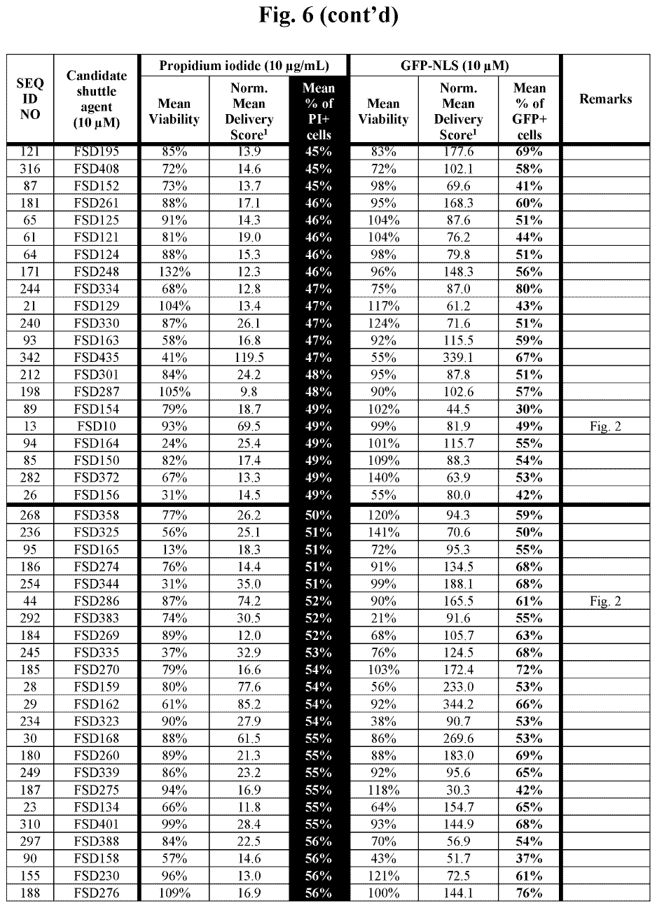

HeLa cells were cultured as described in Example 1.3 and subjected to the PI transduction protocol as described in Example 1.4, with the proteinaceous cargo GFP-NLS being transduced separately as a control in some experiments. Results were acquired by flow cytometry two hours after delivery and expressed as percentages of fluorescent cells (% PI+ cells or % GFP+ cells), as shown in FIGS. 1A-1D and as summarized in the table shown in FIG. 2.

FIGS. 1 and 2 show delivery and viability results of HeLa cells co-incubated for 1 minute with a synthetic peptide shuttle agent or control peptide, combined with either the non-proteinaceous cargo PI (FIGS. 1A and 1B) or the proteinaceous cargo GFP-NLS (FIGS. 1C and 1D). Multiple members of different families of peptide shuttle agents or control peptides were tested. The first group of synthetic peptide shuttle agents tested comprises an endosome leakage domain (ELD) operably linked to a cell penetrating domain (CPD), as previously described in WO/2016/161516 for their ability to transduce proteinaceous cargoes. The second and third groups of synthetic peptide shuttle agents tested correspond to those rationally-designed and optimized for the delivery of proteinaceous cargoes, the second group being peptides previously described in WO/2018/068135. The fourth group of synthetic peptide shuttle agents tested correspond to cyclic peptides possessing either an amide bond between its C and N termini (e.g., “FSD268 cyclic amide”; SEQ ID NO: 49) or a disulfide bridge between two flanking cysteines added in N and C terminal positions (e.g., “FSD268 cyclic disulfide”; SEQ ID NO: 50). The fifth group of peptides are negative control peptides that do not respect several synthetic peptide shuttle agent rational-design parameters described in WO/2018/068135 (e.g., FSN3, FSN4 and FSN8; SEQ ID NOs: 54, 55, and 57, respectively). These negative control peptides also include “FSD10 scramble” (SEQ ID NO: 51), “FSD268 scramble” (SEQ ID NO: 52), and “FSD174 scramble” (SEQ ID NO: 53) peptides having the same amino acid compositions as the peptide shuttle agents FSD10, FSD268, and FSD174, respectively (SEQ ID NOs: 13, 43, and 32, respectively), but in which the order of the amino acids (i.e., the primary amino acid sequence) is changed to deviate from several of the rational-design parameters described in WO/2018/068135. In FIGS. 1A and 1B, “FS then PI” indicates that PI was added 1 hour after the treatment with the synthetic peptide shuttle agents, ensuring that PI-positive signal is not due to cell death. Finally, the right-most bars in FIG. 1A-ID correspond to negative controls in which cells were incubated with cargo alone (“PI” in FIGS. 1A and 1B or “GFP-NLS” in FIGS. 1C and 1D), or untreated cells that were not exposed to the cargo or shuttle peptides (“NT”, FIG. 1A-1D).

Collectively, the results reveal that members of the family of synthetic peptide shuttle agents comprising an ELD operably linked to a CPD (as described in WO/2016/161516), as well as those rationally-designed for the transduction of proteinaceous cargoes (as described in WO/2018/068135), are able to increase the transduction efficiency of a non-proteinaceous, relatively low molecular weight cargo such as PI (in addition to their protein transduction activity). Strikingly, several negative control peptides that fail to respect rational-design parameters described in WO/2018/068135 for the delivery of proteinaceous cargoes also failed to transduce PI, suggesting that the rational-design parameters of WO/2018/068135 may also apply to the design of peptide shuttle agents for the delivery of non-proteinaceous cargoes.

Furthermore, the same synthetic peptide shuttle in linear form (FSD268; SEQ ID NO: 43), in circularized form using amide (FSD268 cyclic amide; SEQ ID NO: 49) or disulfide (FSD268 Cyclic Disulfide; SEQ ID NO: 50) bonds, increased the delivery of PI, confirming that the synthetic shuttle peptides need not be linear to be functional.

### Example 3: Synthetic Peptide Shuttle Agents Enable Intracellular Delivery of Small Molecule Inhibitors of the HedgeHog Signalling Pathway

A rationally-designed peptide shuttle agent, FSD250D (SEQ ID NO: 36), having efficient transduction activity for proteinaceous cargoes, was evaluated for its ability to transduce small molecule inhibitors of the HedgeHog signalling pathway in cultured cells, as described in Example 1.5. The FSD250D peptide has the same amino acid sequence as FSD250 (SEQ ID NO: 35), except that all the amino acids in FSD250D are β-amino acids. Results are shown in FIG. 3 and in Table 1.

TABLE 1
Hedgehog pathway inhibitor delivery to GLI reporter NIH3T3 cells

| | + FSD250D | | − FSD250D | |
|---|---|---|---|---|
| | Mean | | Mean | |
| | luminescence | Standard | luminescence | Standard |
| Conditions | intensity | Deviation | intensity | Deviation |
| Ctrl − mShh | − | − | 0 | 626 |
| Ctrl + mShh | −* | − | 6461 | 773 |
| Gant61 | 4216 | 240 | 6770 | 647 |
| HPI-4 | 1993 | 318 | 6370 | 981 |
| Itraconazole | 1519 | 682 | 5612 | 682 |
| ATO | 4686 | 216 | 5967 | 562 |

*Previous experiments affirmed that the presence of the peptide FSD250D together with mShh did not significantly result in a change in luminescence intensity.

Briefly, the NIH3T3 Gli-luciferase reporter cell line is designed to monitor the activity of the HedgeHog signaling pathway and contains the firefly luciferase gene under the control of Gli responsive elements stably integrated into NIH3T3 cells. As shown in FIG. 3 and Table 1, exposure of the NIH3T3 Gli-luciferase reporter cells to recombinant mouse Sonic HedgeHog protein as a positive control (“Ctrl+mShh”) results in an increase in luminescence intensity that is not observed in the negative control cells which were not exposed to mShh (“Ctrl-mShh”). The presence of the peptide shuttle agent FSD250D had no effect on cellular luminescence intensity following mShh stimulation (data not shown), which was expected given that the receptor for mShh (Patched) is at the cell surface (not intracellular). However, exposure of the reporter cells to structurally different small molecule inhibitors of the HedgeHog signalling pathway that bind to intracellular targets (Gant61, HPI-4, Itraconazole, or ATO) resulted in significantly reduced cellular luminescence intensity in the presence of FSD250D as compared to in the absence of FSD250D, suggesting successful transduction of the small molecules by the peptide shuttle agent. Similar results were observed using the peptide FSD19 (data not shown).

### Example 4: Synthetic Peptide Shuttle Agents Enable Intracellular Delivery of Small Molecule Inhibitors of the HedgeHog Signalling Pathway

A rationally-designed peptide shuttle agent, FSD250D (SEQ ID NO: 36), having efficient transduction activity for proteinaceous cargoes, was evaluated for its ability to transduce small molecule inhibitors of the HedgeHog signalling pathway in a depilated mouse model, as described in Example 1.6.

Briefly, depilation of mouse skin induces hair growth associated with a strong induction of the HedgeHog pathway and increased expression of Gli1. This experiment consisted of activating the HedgeHog pathway in mice by depilation, and then measuring the delay in hair regrowth by delivering in the skin cells small molecule HedgeHog pathway inhibitors that bind to intracellular targets (Gant61 or Itraconazole). The results in FIG. 4 show that mice treated with the small molecule HedgeHog inhibitors Gant61 or Itraconazole (100 μM) in the presence of FSD250D showed delayed hair regrowth at 10 days post-treatment (*), as compared to in the absence of FSD250D.

### Example 5: Synthetic Peptide Shuttle Agents Enable Co-Intracellular Delivery of Small Molecule Sodium Channel Inhibitor (QX-314) and GFP-NLS in HEK293 Cells

The small molecule compound QX-314 (Lidocaine N-ethyl bromide) is a quaternary derivative of lidocaine. QX-314 is not membrane permeable. When delivered to the cell cytoplasm, the QX-314 blocks both fast Na+-dependent action potentials and voltage-dependent, non-inactivating Na+ conductance (Ilfeld and Yaksh, 2009). To evaluate the simultaneous co-transduction of a small molecule and a proteinaceous cargo by peptide shuttle agents, HEK293 cells stably expressing the sodium channel Nav1.7 were exposed to a mixture of QX-314 and GFP-NLS in the presence or absence of the peptide shuttle agent FSD194 (SEQ ID NO: 33). As a control, cells were also treated with GFP-NLS and the peptide shuttle agent FSD194 in the absence of QX-314. Results were evaluated using the patch-clamp technique as described in Example 1.7 and representative whole-cell Na+ currents of the treated HEK293 cells are shown in FIG. 5A-5C. Currents were evoked with a 10 ms depolarizing pulse at 10 Hz. Reduction of the current amplitude was observed when cells were incubated for 90 seconds with QX-314 and GFP-NLS in the presence of the peptide shuttle agent FSD194 (i.e., 1 mM QX-314+15 μM GFP-NLS+5 μM FSD194), consistent with the presence of QX-314 inside the cells (FIG. 5C). In contrast, the same current amplitude reduction was not observed when the cells were incubated without QX-314 (i.e., 15 μM GFP-NLS+5 μM FSD194+; FIG. 5A) or with QX-314 but in the absence of FSD194 (i.e., 2.5 mM QX-314+15 μM GFP-NLS; FIG. 5B). Furthermore, GFP-NLS-positive cells were identified in the QX-314+GFP-NLS+FSD194 and in the FSD194+GFP-NLS conditions, but not in the QX-314+GFP-NLS conditions, indicating that GFP-NLS was indeed co-transduced along with the QX-314 by the peptide shuttle agent.

### Example 6: Robust PI Transduction Predicts Shuttle Agents Having Proteinaceous Cargo Transduction Activity

High-throughput screening efforts to identify, select, and/or qualify novel peptide shuttle agents having protein transduction activity can rapidly become prohibitively expensive due to the high cost of manufacturing and purifying large quantities of recombinant proteins as cargoes, particularly for complex proteins such as recombinant immunoglobulins. The use of GFP or GFP-NLS as a proteinaceous cargo is advantageous, as it enables rapid screening by flow cytometry to assess intracellular delivery. However, the use of GFP-NLS requires verification by microscopy for each peptide shuttle agent, in parallel to flow cytometry measurements, to ensure that the candidate shuttle agent enabled the GFP-NLS cargo to avoid endosomal entrapment and gain access the cytosol/nucleus, which is resource- and time-consuming. Thus, a more cost effective “surrogate” cargo that could reliably predict protein transduction activity and endosomal escape would be highly desirable.

The results in Example 2 demonstrate that synthetic peptide shuttle agents having validated transduction activity for GFP (and other proteinaceous cargoes) can also transduce small molecules such as PI. This raises the intriguing possibility of the converse being true: whether PI can be used as a reliable “surrogate” cargo to screen for and identify/select/qualify novel shuttle agents that possess robust transduction activity for proteinaceous cargoes. Commercially, PI is widely available and relatively inexpensive. Furthermore, PI exhibits 20- to 30-fold enhanced fluorescence and a detectable shift in maximum excitation/emission spectra only after being bound to genomic DNA—a property that makes it particularly suitable to distinguish endosomally-trapped cargo from endosomally-escaped cargo having access to the cytosolic/nuclear compartment. Thus, intracellular delivery and endosomal escape could both be measurable by flow cytometry since any PI that remained trapped in endosomes would not reach the nucleus and would exhibit neither the enhanced fluorescence nor the spectra shift.

To evaluate the suitability of PI as a “surrogate” cargo for novel shuttle agents, a proprietary library of over 300 candidate peptide shuttle agents was screened in parallel for both PI and GFP-NLS transduction activity in HeLa cells using flow cytometry as generally described in Example 1.4. Aside from the concentrations of the cargoes (i.e., 10 μg/mL for PI vs 10 μM for GFP-NLS), the transduction protocols were otherwise the same.

Due to the large number of peptides screened, negative controls were performed in parallel for each experimental batch and included a “no treatment” (NT) control in which the cells were not exposed to shuttle peptide or cargo, as well as a “cargo alone” control in which cells were exposed to the cargo in the absence of shuttle agent. Results are shown in FIGS. 6 and 7, in which “transduction efficiency” refers to the percentage of all viable cells that are positive for the cargo (PI or GFP-NLS). “Mean Delivery score” provides a further indication of the total amount of cargo that was delivered per cell, amongst all cargo-positive cells. Mean PI or GFP-NLS delivery score was calculated by multiplying the mean fluorescence intensity (of at least duplicate samples) measured for the viable PI+ or GFP+ cells by the mean percentage of viable PI+ or GFP+ cells, divided by 100,000 for GFP delivery or by 10,000 for PI delivery. The Mean Delivery Scores for PI and GFP-NLS for each candidate shuttle agent was then normalized by dividing by the Mean Delivery Score for the “cargo alone” negative control performed in parallel for each experimental batch. Thus, the “Norm. Mean Delivery Score” in FIGS. 6 and 7 represents the fold-increase in Mean Delivery Score over the “cargo alone” negative control.

The batch-to-batch variation observed for the negative controls was relatively small for GFP-NLS but was appreciably higher with PI as cargo. For example, the variation in transduction efficiency for the “cargo alone” negative control ranged from 0.4% to 1.3% for GFP-NLS and from 0.9% to 6.3% for PI. Furthermore, transduction efficiencies for several negative control peptides (i.e., peptides known to have low or no GFP transduction activity) tested in parallel (e.g., FSD174 Scramble; data not shown) sometimes gave lower transduction efficiencies for PI (but not for GFP-NLS) than the “cargo alone” negative control, in some cases by as much as 5%, perhaps due to non-specific interactions between PI and the peptides. This phenomenon was not observed for GFP-NLS transduction experiments. The foregoing suggested that the shuttle agent transduction efficiencies at least for PI may be more appropriately compared to that of a negative control peptide rather than to the “cargo alone” condition.

The screening of over 300 candidate peptide shuttle agents for PI and GFP-NLS transduction activity revealed that shuttle agents showing robust transduction efficiency for PI generally correlated with robust transduction efficiency for GFP-NLS. Strikingly, progressively higher PI transduction efficiencies were generally associated with progressively higher GFP-NLS transduction efficiencies. This is illustrated by grouping all the candidate shuttle agents screened into increment windows according to their PI transduction efficiencies and then calculating the average GFP transduction efficiency for all shuttle agents falling within that % PI window, as shown in the table below.

| Mean PI | |
|---|---|
| transduction | Mean GFP transduction |
| efficiency (% PI+) | efficiency (% GFP+) |
| window | within % PI+ window |
| less than 10% | 12% |
| 10-14% | 21% |
| 15-19% | 30% |
| 20-29% | 40% |
| 30-39% | 48% |
| 40-49% | 53% |
| 50-59% | 60% |
| 60-69% | 69% |
| 70-79% | 77% |
| at least 80% | 80% |

FIG. 6 shows results of all candidate peptide shuttle agents screened that had a mean PI transduction efficiency of 10% or higher, sorted based on their level of mean PI transduction efficiency. Strikingly, of the 306 candidate peptide shuttle agents having a mean PI transduction efficiency of at least 10%, 96% of the candidate peptide shuttle agents exhibited GFP transduction efficiencies of 10% or higher. Thresholds of at least 15% and 20% PI transduction efficiency correspond to values of at least 2.5- and 3-fold higher than the highest PI transduction efficiency for the “cargo alone” negative control observed (about 6%) in all experimental batches. Of the 273 candidate peptide shuttle agents listed in FIG. 6 having a mean PI transduction efficiency of at least 15%, 97% of the candidate peptide shuttle agents exhibited GFP transduction efficiencies of 15% or higher. Moreover, of the 256 candidate peptide shuttle agents listed in FIG. 6 having a mean PI transduction efficiency of at least 20%, 99.6% of the candidate peptide shuttle agents exhibited GFP transduction efficiencies of 10% or higher, and 96% of the candidate peptide shuttle agents exhibited GFP transduction efficiencies of 20% or higher.

These results strongly suggest that robust PI delivery predicts peptide shuttle agents having robust proteinaceous cargo transduction activity, and thus that PI can indeed be used as a “surrogate” cargo to screen for and identify/select/qualify novel peptide shuttle agents having dual cargo transducing activity (i.e., for small molecules and proteins).

Included amongst the candidate peptide shuttle agents in FIG. 6 having a mean PI transduction efficiency of at least 20% were peptides having lengths of less than 20 residues: FSD390 (17 aa), FSD367 (19 aa), and FSD366 (18 aa). Also included amongst the candidate peptide shuttle agents in FIG. 6 having a mean PI transduction efficiency of at least 20% were peptides comprising either non-physiological amino acid analogs (e.g., FSD435, which corresponds to FSD395 except for lysine residues (K) being replaced with L-2,4-diaminobutyric acid residues) or chemical modifications (e.g., FSD438, which corresponds to FSD10 except for an N-terminal octanoic acid modification; FSD436, which corresponds to FSD222 except for phenylalanine residues (F) being replaced with (2-naphthyl)-L-alanine residues; FSD171, which corresponds to FSD168 except having an N-terminal acetyl group and a C-terminal cysteamide group. These results confirm the robustness of the peptide shuttle agent platform technology to tolerate the use of non-physiological amino acids or analogs thereof in place of physiological amino acids and/or chemical modifications.

### Example 7: Lower Levels of PI Delivery are Less Predictive of Peptide Shuttle Agents Having Proteinaceous Cargo Transduction Activity

The results of the over 300 candidate peptide shuttle agents screened in Example 6 having a mean PI transduction efficiency of less than 10% but a mean GFP-NLS transduction efficiency of at least 7% are shown in FIG. 7, this time sorted according to their level of mean GFP transduction efficiency.

For candidate peptides having PI transduction efficiencies less than 10%, the large-scale nature of the screening approach employed herein may preclude any firm conclusions as to their potential lack of cargo transduction activity. Indeed, WO/2016/161516 and WO/2018/068135 disclose that shuttle agent peptides function in a concentration-dependent manner and that multiple elements such as shuttle agent concentration, cargo concentration, exposure time, and cell-type may influence shuttle agent performance in transduction assays. The large-scale screening of candidate peptide shuttle agents described herein imposed a “blanket” single shuttle agent concentration, a single cargo concentration, a single exposure time/protocol to each and every peptide tested. Thus, it is difficult to make any firm conclusions as to the non-proteinaceous cargo transduction activity based solely on a low PI transduction efficiency observed in this large-scale screening.

### REFERENCES

Andreu et al., (1992) “Shortened cecropin A-melittin hybrids. Significant size reduction retains potent antibiotic activity”. FEBS letters 296, 190-194
Amand et al., (2012). “Functionalization with C-terminal cysteine enhances transfection efficiency of cell-penetrating peptides through dimer formation.” Biochem Biophys Res Commun 418(3): 469-474.
Boman et al., (1989) Antibacterial and antimalarial properties of peptides that are cecropin-melittin hybrids. FEBS letters 259, 103-106.
Drin et al., (2003). “Studies on the internalization mechanism of cationic cell-penetrating peptides.” J Biol Chem 278(33): 31192-31201.
Eisenberg et al., (1982). “The helical hydrophobic moment: a measure of the amphiphilicity of a helix”. Nature 299, 371-374.
El-Andaloussi et al., (2007). “A novel cell-penetrating peptide, M918, for efficient delivery of proteins and peptide nucleic acids.” Mol Ther 15(10): 1820-1826.
El-Sayed et al., (2009). “Delivery of macromolecules using arginine-rich cell-penetrating peptides: ways to overcome endosomal entrapment.” AAPSJ 11(1): 13-22.
Elmquist et al., (2001). “VE-cadherin-derived cell-penetrating peptide, pVEC, with carrier functions.” Exp Cell Res 269(2): 237-244.
Erazo-Oliveras et al., (2014) “Protein delivery into live cells by incubation with an endosomolytic agent.” Nat Methods. (8):861-7.
Fawell et al., (1994). “Tat-mediated delivery of heterologous proteins into cells.” Proc Natl Acad Sci USA 91(2): 664-668.
Fominaya et al., (1998). “A chimeric fusion protein containing transforming growth factor-alpha mediates gene transfer via binding to the EGF receptor.” Gene Ther 5(4): 521-530.
Fominaya, J. and W. Wels (1996). “Target cell-specific DNA transfer mediated by a chimeric multidomain protein. Novel non-viral gene delivery system.” J Biol Chem 271(18): 10560-10568.
Glover et al., (2009). “Multifunctional protein nanocarriers for targeted nuclear gene delivery in nondividing cells.” FASEB J 23(9): 2996-3006.
Gottschalk et al., (1996). “A novel DNA-peptide complex for efficient gene transfer and expression in mammalian cells.” Gene Ther 3(5): 448-457.
Green, M. and P. M. Loewenstein (1988). “Autonomous functional domains of chemically synthesized human immunodeficiency virus tat trans-activator protein.” Cell 55(6): 1179-1188.
Hallbrink et al., (2001). “Cargo delivery kinetics of cell-penetrating peptides.” Biochim Biophys Acta 1515(2): 101-109.
Herce, H. D. and A. E. Garcia (2007). “Molecular dynamics simulations suggest a mechanism for translocation of the HIV-1 TAT peptide across lipid membranes.” Proc Natl Acad Sci USA 104(52): 20805-20810.
Ho et al., (2001). “Synthetic protein transduction domains: enhanced transduction potential in vivo.” Cancer Research 61: 474-477.
Ilfeld and Yaksh (2009). “The End of Postoperative Pain—A Fast-Approaching Possibility? And, if So, Will We Be Ready?” Regional Anesthesia and Pain Medicine 34(2): 85-87.
Kakudo et al., (2004). “Transferrin-modified liposomes equipped with a pH-sensitive fusogenic peptide: an artificial viral-like delivery system.” Biochemistry 43(19): 5618-5628.
Kichler et al., (2006). “Cationic amphipathic histidine-rich peptides for gene delivery.” Biochim Biophys Acta 1758(3): 301-307.
Kichler et al., (2003). “Histidine-rich amphipathic peptide antibiotics promote efficient delivery of DNA into mammalian cells”. Proc Natl Acad Sci USA. 2003 Feb. 18; 100(4): 1564-1568.
Kwon, et al., (2010). “A Truncated HGP Peptide Sequence That Retains Endosomolytic Activity and Improves Gene Delivery Efficiencies”. Mol. Pharmaceutics, 7:1260-65.
Lamiable et al., (2016). “PEP-FOLD3: faster de novo structure prediction for linear peptides in solution and in complex” Nucleic Acids Res. 44(W1):W449-54.
Li et al., (2004). “GALA: a designed synthetic pH-responsive amphipathic peptide with applications in drug and gene delivery.” Adv Drug Deliv Rev 56(7): 967-985.
London, E. (1992). “Diphtheria toxin: membrane interaction and membrane translocation.” Biochim Biophys Acta 1113(1): 25-51.
Lorieau et al., (2010). “The complete influenza hemagglutinin fusion domain adopts a tight helical hairpin arrangement at the lipid:water interface.” Proc Natl Acad Sci USA 107(25): 11341-11346.
Luan et al., (2015). “Peptide amphiphiles with multifunctional fragments promoting cellular uptake and endosomal escape as efficient gene vectors.” J Mater. Chem. B, 3: 1068-1078.
Mahlum et al., (2007). “Engineering a noncarrier to a highly efficient carrier peptide for noncovalently delivering biologically active proteins into human cells.” Anal Biochem 365(2): 215-221.
Midoux et al., (1998). “Membrane permeabilization and efficient gene transfer by a peptide containing several histidines.” Bioconjug Chem 9(2): 260-267.
Montrose et al., (2013). “Xentry, a new class of cell-penetrating peptide uniquely equipped for delivery of drugs.” Sci Rep 3: 1661.
Morris, M. C., L. Chaloin, M. Choob, J. Archdeacon, F. Heitz and G. Divita (2004). “Combination of a new generation of PNAs with a peptide-based carrier enables efficient targeting of cell cycle progression.” Gene Ther 11(9): 757-764.
Morris et al., (2001). “A peptide carrier for the delivery of biologically active proteins into mammalian cells.” Nat Biotechnol 19(12): 1173-1176.
O'Keefe, D. O. (1992). “Characterization of a full-length, active-site mutant of diphtheria toxin.” Arch Biochem Biophys 296(2): 678-684.
Parente et al., (1990). “Mechanism of leakage of phospholipid vesicle contents induced by the peptide GALA.” Biochemistry 29(37): 8720-8728.
Perez et al., (1992). “Antennapedia homeobox as a signal for the cellular internalization and nuclear addressing of a small exogenous peptide.” J Cell Sci 102 (Pt 4): 717-722.
Salomone et al., (2012). “A novel chimeric cell-penetrating peptide with membrane-disruptive properties for efficient endosomal escape.” J Control Release 163(3): 293-303.
Schuster et al., “Multicomponent DNA carrier with a vesicular stomatitis virus G-peptide greatly enhances liver-targeted gene expression in mice.” Bioconjug Chem 10(6): 1075-1083.
Shaw et al., (2008). “Comparison of protein transduction domains in mediating cell delivery of a secreted CRE protein.” Biochemistry 47(4): 1157-1166.
Shen et al., (2014) “Improved PEP-FOLD approach for peptide and miniprotein structure prediction”. J Chem. Theor. Comput. 10:4745-4758.
Tan et al., (2012). “Truncated peptides from melittin and its analog with high lytic activity at endosomal pH enhance branched polyethylenimine-mediated gene transfection.” J Gene Med 14(4): 241-250.
Thdriault et al., “Differential modulation of Nav1.7 and Nav1.8 channels by antidepressant drugs.” European Journal of Pharmacology (2015) 764: 395-403.
Thévenet et al., “PEP-FOLD: an updated de novo structure prediction server for both linear and disulfide bonded cyclic peptides.” Nucleic Acids Res. 2012. 40, W288-293.
Uherek et al., (1998). “A modular DNA carrier protein based on the structure of diphtheria toxin mediates target cell-specific gene delivery.” J Biol Chem 273(15): 8835-8841.
Varkouhi et al., “Endosomal escape pathways for delivery of biologicals.” J Control Release 151(3): 220-228.
Veach et al., (2004). “Receptor/transporter-independent targeting of functional peptides across the plasma membrane.” J Biol Chem 279(12): 11425-11431.
Vives et al., (1997). “A truncated HIV-1 Tat protein basic domain rapidly translocates through the plasma membrane and accumulates in the cell nucleus.” J Biol Chem 272(25): 16010-16017.<
Wyman et al., (1997). “Design, synthesis, and characterization of a cationic peptide that binds to nucleic acids and permeabilizes bilayers.” Biochemistry 36(10): 3008-3017.
Zhou et al., (2009). “Generation of induced pluripotent stem cells using recombinant proteins.” Cell Stem Cell 4(5): 381-384.

1-58. (canceled)

59. A synthetic peptide shuttle agent having transduction activity for both proteinaceous and non-proteinaceous cargoes in target eukaryotic cells, the synthetic peptide shuttle agent comprising:
an amino acid sequence at least 70% identical to the full length of SEQ ID NO: 285; or
an amino acid sequence that differs from SEQ ID NO: 285 by no more than 8 amino acids,
wherein the synthetic peptide shuttle agent is a peptide at least 17 amino acids in length comprising an amphipathic alpha-helical motif comprising a positively-charged hydrophilic outer face and a hydrophobic outer face, and the synthetic peptide shuttle agent comprises at least five of the following parameters:
(a) the hydrophobic outer face comprises a highly hydrophobic core consisting of spatially adjacent L, I, F, V, W, and/or M amino acids representing 12% to 50% of the amino acids of the peptide, based on an open cylindrical representation of the alpha-helix having 3.6 residues per turn;
(b) the peptide has a hydrophobic moment (μ) of 3.5 to 11;
(c) the peptide has a predicted net charge of at least +4 at physiological pH;
(d) the peptide has an isoelectric point (pI) of 8 to 13;
(e) the peptide is composed of 35% to 65% of any combination of the amino acids: A, C, G, I, L, M, F, P, W, Y, and/or V;
(f) the peptide is composed of 0% to 30% of any combination of the amino acids: N, Q, S, and/or T;
(g) the peptide is composed of 35% to 85% of any combination of the amino acids: A, L, K, and/or R;
(h) the peptide is composed of 15% to 45% of any combination of the amino acids: A and/or L, provided there being at least 5% of L in the peptide;
(i) the peptide is composed of 20% to 45% of any combination of the amino acids: K and/or R;
(j) the peptide is composed of 0% to 10% of any combination of the amino acids: D and/or E;
(k) the difference between the percentage of A and L residues in the peptide (% A+L), and the percentage of K and R residues in the peptide (% K+R), is less than or equal to 10%; and/or
(l) the peptide is composed of 10% to 45% of any combination of the amino acids: Q, Y, W, P, I, S, G, V, F, E, D, C, M, N, T and/or H,
wherein the synthetic peptide shuttle agent increases the transduction efficiency of propidium iodide or other membrane-impermeable fluorescent DNA intercalating agents by at least 3-fold over a corresponding negative control lacking said synthetic peptide shuttle agent, and/or enables a transduction efficiency of at least 10% of propidium iodide or other membrane-impermeable fluorescent DNA intercalating agents, in a eukaryotic cell line model suitable for assessing cargo transduction in said target eukaryotic cells;
wherein the synthetic peptide shuttle agent increases the transduction efficiency of GFP-NLS by at least 3-fold over a corresponding negative control lacking said synthetic peptide shuttle agent, and/or enables a transduction efficiency of at least 7% of GFP-NLS, in a eukaryotic cell line model suitable for assessing cargo transduction in said target eukaryotic cells.

60. The synthetic peptide shuttle agent of claim 59, wherein:
(a) the synthetic peptide shuttle agent is a peptide having a maximum length of 150 amino acids;
(b) said amphipathic alpha-helical motif has a hydrophobic moment (μ) between 3.5 and 11.0;
(c) said amphipathic alpha-helical motif comprises a positively-charged hydrophilic outer face comprising: (i) at least two adjacent positively-charged K and/or R residues upon helical wheel projection; and/or (ii) a segment of six adjacent residues comprising three to five K and/or R residues upon helical wheel projection, based on an alpha helix having angle of rotation between consecutive amino acids of 100 degrees and/or an alpha-helix having 3.6 residues per turn;
(d) said amphipathic alpha-helical motif comprises a hydrophobic outer face comprising: (i) at least two adjacent L residues upon helical wheel projection; and/or (ii) a segment of ten adjacent residues comprising at least five hydrophobic residues selected from: L, I, F, V, W, and M, upon helical wheel projection, based on an alpha helix having angle of rotation between consecutive amino acids of 100 degrees and/or an alpha-helix having 3.6 residues per turn;
(e) said hydrophobic outer face comprises a highly hydrophobic core consisting of spatially adjacent L, I, F, V, W, and/or M amino acids representing from 12.5%, 13%, 13.5%, 14%, 14.5%, 15%, 15.5%, 16%, 16.5%, 17%, 17.5%, 18%, 18.5%, 19%, 19.5%, or 20%, to 25%, 30%, 35%, 40%, or 45% of the amino acids of the synthetic peptide shuttle agent;
(f) the synthetic peptide shuttle agent has a hydrophobic moment (μ) between 4.0 and 10.5;
(g) the synthetic peptide shuttle agent has a predicted net charge of +4 to +15; or;
(h) the synthetic peptide shuttle agent has a predicted pI of 10 to 13; or
(i) any combination of (a) to (h).

61. The synthetic peptide shuttle agent of claim 59, wherein said synthetic peptide shuttle agent comprises at least one, at least two, at least three, at least four, at least five, at least six, or all of the following parameters:
(a) the synthetic peptide shuttle agent is composed of 36% to 64% of any combination of the amino acids: A, C, G, I, L, M, F, P, W, Y, and/or V;
(b) the synthetic peptide shuttle agent is composed of 1% to 29% of any combination of the amino acids: N, Q, S, and/or T;
(c) the synthetic peptide shuttle agent is composed of 36% to 80% of any combination of the amino acids: A, L, K, and/or R;
(d) the synthetic peptide shuttle agent is composed of 15% to 40% of any combination of the amino acids: A and/or L;
(e) the synthetic peptide shuttle agent is composed of 20% to 40% of any combination of the amino acids: K and/or R;
(f) the synthetic peptide shuttle agent is composed of 5% to 10% of any combination of the amino acids: D and/or E;
(g) the difference between the percentage of A and L residues in the synthetic peptide shuttle agent (% A+L), and the percentage of K and R residues in the synthetic peptide shuttle agent (% K+R), is less than or equal to 9%; and/or
(h) the synthetic peptide shuttle agent is composed of 15% to 40% of any combination of the amino acids: Q, Y, W, P, I, S, G, V, F, E, D, C, M, N, T, and/or H.

62. The synthetic peptide shuttle agent of claim 59, wherein the synthetic peptide shuttle agent comprises or consists of an amino acid sequence that differs from SEQ ID NO: 285 by no more than 7 amino acids.

63. The synthetic peptide shuttle agent of claim 59, wherein the synthetic peptide shuttle agent comprises or consists of an amino acid sequence that differs from SEQ ID NO: 285 by no more than 6 amino acids.

64. The synthetic peptide shuttle agent of claim 59, wherein the synthetic peptide shuttle agent comprises or consists of an amino acid sequence that differs from SEQ ID NO: 285 by no more than 5 amino acids.

65. The synthetic peptide shuttle agent of claim 59, wherein the synthetic peptide shuttle agent comprises or consists of an amino acid sequence that differs from SEQ ID NO: 285 by no more than 4 amino acids.

66. The synthetic peptide shuttle agent of claim 59, wherein the synthetic peptide shuttle agent comprises or consists of an amino acid sequence that differs from SEQ ID NO: 285 by no more than 3 amino acids.

67. The synthetic peptide shuttle agent of claim 59, wherein the synthetic peptide shuttle agent comprises or consists of an amino acid sequence that differs from SEQ ID NO: 285 by no more than 2 amino acids.

68. The synthetic peptide shuttle agent of claim 59, wherein the synthetic peptide shuttle agent comprises or consists of an amino acid sequence that is at least 90% identical to the full length of SEQ ID NO: 285.

69. The synthetic peptide shuttle agent of claim 59, wherein the synthetic peptide shuttle agent comprises or consists of an amino acid sequence that is at least 95% identical to the full length of SEQ ID NO: 285.

70. The synthetic peptide shuttle agent of claim 59, wherein the synthetic peptide shuttle agent is a cyclic peptide and/or comprises one or more D-amino acids.

71. The synthetic peptide shuttle agent of claim 59, further comprising a chemical modification to one or more amino acids, wherein the chemical modification is the addition of an acetyl group, a cysteamide group, or a fatty acid, at the N and/or C terminus of the synthetic peptide shuttle agent.

72. A synthetic peptide shuttle agent having transduction activity for both proteinaceous and non-proteinaceous cargoes in target eukaryotic cells, wherein the synthetic peptide shuttle agent comprises or consists of an amino acid sequence that differs from SEQ ID NO: 285 by only conservative amino acid substitutions and comprises an amphipathic alpha-helical motif comprising a positively-charged hydrophilic outer face and a hydrophobic outer face, wherein the synthetic peptide shuttle agent: increases the transduction efficiency of propidium iodide or other membrane-impermeable fluorescent DNA intercalating agents by at least 3-fold over a corresponding negative control lacking said synthetic peptide shuttle agent, and enables a transduction efficiency of at least 10% of propidium iodide or other membrane-impermeable fluorescent DNA intercalating agents, in a eukaryotic cell line model suitable for assessing cargo transduction in said target eukaryotic cells, and wherein each conservative amino acid substitution is selected from an amino acid within the same amino acid class, the amino acid class being: Aliphatic: G, A, V, L, and I; Hydroxyl or sulfur/selenium-containing: S, C, U, T, and M; Aromatic: F, Y, and W; Basic: H, K, and R; Acidic and amides thereof: D, E, N, and Q.

73. The synthetic peptide shuttle agent of claim 72, wherein the synthetic peptide shuttle agent is a cyclic peptide and/or comprises one or more D-amino acids.

74. The synthetic peptide shuttle agent of claim 72, further comprising a chemical modification to one or more amino acids, wherein the chemical modification is the addition of an acetyl group, a cysteamide group, or a fatty acid, at the N and/or C terminus of the synthetic peptide shuttle agent.

75. A synthetic peptide shuttle agent having transduction activity for both proteinaceous and non-proteinaceous cargoes in target eukaryotic cells, wherein the synthetic peptide shuttle agent comprises or consists of the amino acid sequence of SEQ ID NO: 285.

76. The synthetic peptide shuttle agent of claim 75, wherein the synthetic peptide shuttle agent is a cyclic peptide and/or comprises one or more D-amino acids.

77. The synthetic peptide shuttle agent of claim 75, further comprising a chemical modification to one or more amino acids, wherein the chemical modification is the addition of an acetyl group, a cysteamide group, or a fatty acid, at the N and/or C terminus of the synthetic peptide shuttle agent.

78. A synthetic peptide shuttle agent variant having transduction activity for both proteinaceous and non-proteinaceous cargoes in target eukaryotic cells, the synthetic peptide shuttle agent variant being identical to the synthetic peptide shuttle agent as defined in claim 74, except having at least one amino acid being replaced with a corresponding synthetic amino acid, wherein the synthetic peptide shuttle agent variant increases the transduction efficiency of said cargo in the target eukaryotic cells, as compared to in the absence of the synthetic peptide shuttle agent variant; and wherein the synthetic amino acid:
(a) replaces a basic amino acids with any one of: α-aminoglycine, α,γ-diaminobutyric acid, ornithine, α,β-diaminopropionic acid, 2,6-diamino-4-hexynoic acid, β-(1-piperazinyl)-alanine, 4,5-dehydro-lysine, δ-hydroxylysine, ω,ω-dimethylarginine, homoarginine, ω,ω′-dimethylarginine, ω-methylarginine, β-(2-quinolyl)-alanine, 4-aminopiperidine-4-carboxylic acid, α-methylhistidine, 2,5-diiodohistidine, 1-methylhistidine, 3-methylhistidine, spinacine, 4-aminophenylalanine, 3-aminotyrosine, β-(2-pyridyl)-alanine, or β-(3-pyridyl)-alanine;
(b) replaces a non-polar (hydrophobic) amino acid with any one of: dehydro-alanine, β-fluoroalanine, β-chloroalanine, β-lodoalanine, α-aminobutyric acid, α-aminoisobutyric acid, β-cyclopropylalanine, azetidine-2-carboxylic acid, α-allylglycine, propargylglycine, tert-butylalanine, β-(2-thiazolyl)-alanine, thiaproline, 3,4-dehydroproline, tert-butylglycine, β-cyclopentylalanine, β-cyclohexylalanine, α-methylproline, norvaline, α-methylvaline, penicillamine, β,β-dicyclohexylalanine, 4-fluoroproline, 1-aminocyclopentanecarboxylic acid, pipecolic acid, 4,5-dehydroleucine, allo-isoleucine, norleucine, α-methylleucine, cyclohexylglycine, cis-octahydroindole-2-carboxylic acid, β-(2-thienyl)-alanine, phenylglycine, α-methylphenylalanine, homophenylalanine, 1,2,3,4-tetrahydroisoquinoline-3-carboxylic acid, β-(3-benzothienyl)-alanine, 4-nitrophenylalanine, 4-bromophenylalanine, 4-tert-butylphenylalanine, α-methyltryptophan, β-(2-naphthyl)-alanine, β-(1-naphthyl)-alanine, 4-iodophenylalanine, 3-fluorophenylalanine, 4-fluorophenylalanine, 4-methyltryptophan, 4-chlorophenylalanine, 3,4-dichloro-phenylalanine, 2,6-difluoro-phenylalanine, n-in-methyltryptophan, 1,2,3,4-tetrahydronorharman-3-carboxylic acid, β,β-diphenylalanine, 4-methylphenylalanine, 4-phenylphenylalanine, 2,3,4,5,6-pentafluoro-phenylalanine, or 4-benzoylphenylalanine;
(c) replaces a polar, uncharged amino acid with any one of: β-cyanoalanine, β-ureidoalanine, homocysteine, allo-threonine, pyroglutamic acid, 2-oxothiazolidine-4-carboxylic acid, citrulline, thiocitrulline, homocitrulline, hydroxyproline, 3,4-dihydroxyphenylalanine, β-(1,2,4-triazol-1-yl)-alanine, 2-mercaptohistidine, β-(3,4-dihydroxyphenyl)-serine, β-(2-thienyl)-serine, 4-azidophenylalanine, 4-cyanophenylalanine, 3-hydroxymethyltyrosine, 3-iodotyrosine, 3-nitrotyrosine, 3,5-dinitrotyrosine, 3,5-dibromotyrosine, 3,5-diiodotyrosine, 7-hydroxy-1,2,3,4-tetrahydroiso-quinoline-3-carboxylic acid, 5-hydroxytryptophan, thyronine, β-(7-methoxycoumarin-4-yl)-alanine, or 4-(7-hydroxy-4-coumarinyl)-aminobutyric acid; and/or
(d) replaces an acidic amino acid with any one of: γ-hydroxyglutamic acid, γ-methyleneglutamic acid, γ-carboxyglutamic acid, α-aminoadipic acid, 2-aminoheptanedioic acid, α-aminosuberic acid, 4-carboxyphenylalanine, cysteic acid, 4-phosphonophenylalanine, or 4-sulfomethylphenylalanine.
